SEQUENCE LISTING (4 sequences)

SEQ ID NO: 1 - DNA, 45 bp, synthetic construct
  source 1..45: mol_type = other DNA; note = linker
  misc_feature 1: note = 5' modification - 5AmMC12
  modified_base 31: mod_base = OTHER; note = deoxyU
  modified_base 33: mod_base = OTHER; note = deoxyU
  modified_base 35: mod_base = OTHER; note = deoxyU
  modified_base 37: mod_base = OTHER; note = deoxyU
  misc_feature 45: note = 3' modification - 3ThioMC3-D
ttttttttcccttcctcctcacttccctttttttttttttttttt

SEQ ID NO: 2 - DNA, 45 bp, synthetic construct; identical to SEQ ID NO: 1
  source 1..45: mol_type = other DNA; note = linker
  misc_feature 1: note = 5' modification - 5AmMC12
  modified_base 31: mod_base = OTHER; note = deoxyU
  modified_base 33: mod_base = OTHER; note = deoxyU
  modified_base 35: mod_base = OTHER; note = deoxyU
  modified_base 37: mod_base = OTHER; note = deoxyU
  misc_feature 45: note = 3' modification - 3ThioMC3-D

SEQ ID NO: 3 - DNA, 67 bp, synthetic construct
  source 1..67: mol_type = other DNA; note = linker
  misc_feature 1: note = 5' modification - 5AmMC12
  modified_base 31: mod_base = OTHER; note = deoxyU
  misc_feature 31..>59: note = deoxyUT repeats and some of the repeats can be excluded
  modified_base 33: mod_base = OTHER; note = deoxyU
  modified_base 35: mod_base = OTHER; note = deoxyU
  modified_base 37: mod_base = OTHER; note = deoxyU
  modified_base 39: mod_base = OTHER; note = deoxyU
  modified_base 41: mod_base = OTHER; note = deoxyU
  modified_base 43: mod_base = OTHER; note = deoxyU
  modified_base 45: mod_base = OTHER; note = deoxyU
  modified_base 47: mod_base = OTHER; note = deoxyU
  modified_base 49: mod_base = OTHER; note = deoxyU
  modified_base 51: mod_base = OTHER; note = deoxyU
  modified_base 53: mod_base = OTHER; note = deoxyU
  modified_base 55: mod_base = OTHER; note = deoxyU
  modified_base 57: mod_base = OTHER; note = deoxyU
  modified_base 59: mod_base = OTHER; note = deoxyU
  misc_feature 67: note = 3' modification - 3ThioMC3-D
ttttttttcccttcctcctcacttccctttttttttttttttttttttttttttttttttttttttt

SEQ ID NO: 4 - DNA, 45 bp, synthetic construct; identical to SEQ ID NO: 1
  source 1..45: mol_type = other DNA; note = linker
  misc_feature 1: note = 5' modification - 5AmMC12
  modified_base 31: mod_base = OTHER; note = deoxyU
  modified_base 33: mod_base = OTHER; note = deoxyU
  modified_base 35: mod_base = OTHER; note = deoxyU
  modified_base 37: mod_base = OTHER; note = deoxyU
  misc_feature 45: note = 3' modification - 3ThioMC3-D

(19) United States
(12) Patent Application Publication
(10) Pub. No.: US 2023/0384337 A1
(43) Pub. Date: Nov. 30, 2023

(54) High Efficiency Capture of Fetal Cells from Maternal Samples; and Whole Blood Buffer Compositions and Related Methods

(71) Applicant: BioFluidica, Inc., San Diego, CA (US)

(72) Inventors: Rolf Muller, Del Mar, CA (US); Judy Muller-Cohn, Del Mar, CA (US); Philip D. Buchanan, Chapel Hill, NC (US); Jiri Sonek, Beavercreek, OH (US); Kulvinder Kaur, Carlsbad, CA (US); Jennifer Barber-Singh, San Diego, CA (US); Paul Diaz, Riverside, CA (US)

(21) Appl. No.: 18/226,733

(22) Filed: Jul. 26, 2023

Related U.S. Application Data
(63) Continuation-in-part of application No. PCT/US2022/014168, filed on Jan. 27, 2022.
(60) Provisional application No. 63/142,288, filed on Jan. 27, 2021.
(60) Provisional application No. 63/394,227, filed on Aug. 1, 2022.

(51) Int. Cl. G01N 35/10; G01N 33/50

(52) U.S. Cl. CPC G01N 35/1065; G01N 33/5005

(57) ABSTRACT

The presently disclosed subject matter provides methods of isolating fetal cells from a sample from a pregnant subject, methods of isolating multinucleated fetal giant cells from a sample from a pregnant subject, and related compositions and methods.

### CROSS REFERENCE TO RELATED APPLICATIONS

This application is a continuation-in-part application of PCT/US2022/014168 filed Jan. 27, 2022, which claims priority to U.S. application Ser. No. 63/142,288 filed Jan. 27, 2021; and claims priority to U.S. application Ser. No. 63/394,227 filed Aug. 1, 2022. The contents of each are hereby incorporated by references in their entirety.

### STATEMENT REGARDING FEDERALLY SPONSORED RESEARCH OR DEVELOPMENT

This invention was made with government support under Grant No. R43HD098942-01, awarded by the Eunice Kennedy Shriver National Institute of Child Health & Human Development, an institute of the National Institutes of Health. The United States has certain rights in the invention.

### FIELD

The invention relates to the fields of non-invasive prenatal screening and diagnostics; isolation and recovery of fetal biomarker analytes, including polynucleated fetal giant cells and cytotrophoblasts, from maternal blood; and whole blood buffer compositions and related methods for sample preparation and methods for eliminating microclotting and aggregation for microfluidic applications.

### SEQUENCE LISTING

The instant application contains a Sequence Listing which has been submitted electronically in ASCII text file format and is hereby incorporated by reference in its entirety. Said ASCII copy, created Jan. 27, 2022 with PatentIn software, is named “Sequence_Listing_998-3-1” and is 3.46 KB in size.

### BACKGROUND

About 20 pregnancies in 1000 involve a genetic disability. About fifty percent are copy number abnormalities (including autosomal and sex chromosome aneuploidies and microdeletions and duplications) and about fifty percent are single gene disorders. Trisomy 21, while being the most common genetic disability, only accounts for about five percent of all genetic disabilities.

Non-invasive prenatal screening requires a sufficient number of fetal cells to be isolated from each sample for down-stream analysis. While prenatal testing based on fetal cells isolated from a maternal sample from a pregnant subject have been developed, there remains a need for improved fetal cell isolation to extract more fetal cells or fetal DNA per sample and for improved transition from fetal cell isolation to cell analysis.

### SUMMARY OF INVENTION

The presently disclosed subject matter describes a method of isolating fetal cells from a sample from a pregnant subject comprising (a) providing an isolation system comprising one or more microfluidic chips, wherein each microfluidic chip comprises i) an inlet port, ii) an outlet port, and iii) multiple parallel sinusoidal microchannels in fluid communication with the inlet port and outlet port; wherein said sinusoidal microchannels comprise at least one of: binding moieties that selectively bind to EpCAM, binding moieties that selectively bind to CD105, and/or binding moieties that selectively bind to CD141; a liquid handling system comprising i) a pair of automated pipettes corresponding to each microfluidic chip, comprising a) a first automated pipette comprising a first pump, and a first pipette tip containing a sample from a pregnant subject and coupled to the inlet port, wherein the sample is maternal whole blood, has not been processed to remove maternal cells, and comprises at least one fetal cell and; b) a second automated pipette comprising a second pump, and a second pipette tip coupled to the outlet port; and ii) a controller comprising a non-transitory computer readable medium in communication with the first pump and the second pump, and programmed to command the first pump of the first automated pipette and the second pump of the second automated pipette to control flow of the sample through said one or more microfluidic chips. The method further comprises (b) hydrodynamically processing the sample through said one or more microfluidic chips; and (c) isolating at least one fetal cell, wherein the at least one fetal cell contacts at least one of said binding moieties, and wherein the method does not comprise removing maternal cells prior to isolation of the at least one fetal cell.

In some embodiments, the isolation system comprises two or more microfluidic chips, wherein each first pipette tip of each first automated pipette of each pair of automate pipettes corresponding to each chip comprises the same sample, wherein hydrodynamically processing the sample through said two or more microfluidic chips comprises flowing a portion of the same sample through each microfluidic chip, and the method comprises isolating an average of 0.5 fetal cells per milliliter of sample. In some embodiments, the method comprises recovering at least 1 fetal cell in the presence of at least 500 maternal cells, at least 750 maternal cells, or at least 1000 maternal cells. In some embodiments, the method comprises recovering at least 1 fetal cell in the presence of 10-1,000 maternal cells. In some embodiments, the isolation system comprises eight microfluidic chips and eight pairs of automated pipettes with each respective first pipette tip containing at least 1 milliliter of the same sample, and wherein hydrodynamically processing the sample through said eight microfluidic chips comprises flowing the at least lml of sample in each first pipette tip through the corresponding microfluidic chip. In some embodiments, the binding moieties are antibodies, aptamers, affimers, or haptens immobilized to the interior surface of the sinusoidal microchannels.

In some embodiments, the microchannels comprise a synergistic combination of binding moieties that selectively bind to EpCAM and binding moieties that selectively bind to CD141. In some embodiments, the method comprises isolating at least 3 fetal cells per milliliter of sample. In some embodiments, the isolation system comprises two or more microfluidic chips, wherein each first pipette tip of each first automated pipette of each pair of automate pipettes corresponding to each chip comprises the same sample, wherein hydrodynamically processing the sample through said two or more microfluidic chips comprises flowing a portion of the same sample through each microfluidic chip, and the method comprises isolating at least an average of 3 fetal cells per milliliter of sample.

In some embodiments, the microchannels comprise a synergistic combination of binding moieties that selectively bind to EpCAM and binding moieties that selectively bind to CD105. In some embodiments, the method comprises isolating at least 2 fetal cells per milliliter of sample. In some embodiments, the isolation system comprises two or more microfluidic chips, wherein each first pipette tip of each first automated pipette of each pair of automate pipettes corresponding to each chip comprises the same sample, wherein hydrodynamically processing the sample through said two or more microfluidic chips comprises flowing a portion of the same sample through each microfluidic chip, and the method comprises isolating at least an average of 2 fetal cells per milliliter of sample.

In some embodiments, the fetal cells are fetal trophoblastic cells. In some embodiments, the fetal cells are extravillous trophoblast cells. In some embodiments, the sample is taken during gestation week 8, 9, 10, 11, 12, 13, 14, 15, 16, 17, or 18. In some embodiments, the microchannels have a microchannel width in the range of about 15 μm to about 45 μm and a microchannel height in the range of about 100 μm to about 160 μm.

In some embodiments, the sample is maternal whole blood admixed with a whole blood buffer composition comprising a ficoll polysaccharide and a halogenated sucrose derivative. In some embodiments, the sample is maternal whole blood admixed with a dual whole blood and fixative buffer composition comprising a ficoll polysaccharide, a halogenated sucrose derivative, an anticoagulant, at least one of glyoxal or paraformaldehyde or formaldehyde or a formal acetic acid or a formal saline or a phosphate formalin or a formalin calcium, and a pH buffer.

In some embodiments, the hydrodynamically processing the sample through said one or more microfluidic chips comprises applying a pulsative flow. In some embodiments, the overall flow rate is in the range of 50 to 300 per minute, 100 to 250 per minute, 100 to30 ill per minute, 50 to 150 per minute, 50 to 100 per minute, or 100 to 150 per minute.

The presently disclosed subject matter describes a method of isolating multinucleated fetal giant cells from a sample from a pregnant subject comprising: (a) providing an isolation system comprising one or more microfluidic chips, wherein each microfluidic chip comprises i) inlet port, ii) an outlet port, and iii) multiple parallel sinusoidal microchannels in fluid communication with the inlet port and outlet port; wherein said sinusoidal microchannels have a channel width in the range of about 15 μm to about 45 μm and a channel height in the range of about 100 μm to about 150 μm, and wherein said sinusoidal microchannels comprise binding moieties that selectively bind to a surface marker of fetal cells; a liquid handling system comprising i) a pair of automated pipettes corresponding to each microfluidic chip, comprising a) first automated pipette comprising a first pump, and a first pipette tip containing a sample from a pregnant subject and coupled to the inlet port, wherein the sample from a pregnant subject is maternal whole blood and comprises at least one multinucleated fetal giant cell; b) a second automated pipette comprising a second pump, and a second pipette tip coupled to the outlet port; and ii) a controller comprising a non-transitory computer readable medium in communication with the first pump and the second pump, and programmed to command the first pump of the first automated pipette and the second pump of the second automated pipette to control flow of the sample through said one or more microfluidic chips. The method further comprises (b) hydrodynamically processing the sample through said one or more microfluidic chips; and (c) isolating at least one multinucleated fetal giant cell, wherein the at least one multinucleated fetal giant cell contacts at least one of said binding moieties and wherein the at least one multinucleated fetal giant cell is 50 to 150 μm in diameter as measured along the longest axis.

In some embodiments, the sinusoidal microchannels have a channel width in the range of about 20 μm to about 25 μm and a channel height in the range of about 140 μm to about 150 m. In some embodiments, the sample has not been processed to removed maternal cells, and the method does not comprise removing maternal cells prior to isolation of the at least one fetal cell.

In some embodiments, the hydrodynamically processing the sample through said one or more microfluidic chips comprises applying a pulsative flow. In some embodiments, the overall flow rate is in the range of 50 to 300 per minute, 100 to 250 per minute, 100 to30 ill per minute, 50 to 150 per minute, 50 to 100 per minute, or 100 to 150 per minute. In some embodiments, applying a pulsative flow comprises 300 pulses per milliliter of maternal sample and a repetitive flow pattern comprising an increase in flow rate to 1 μl/sec for 100 milliseconds and a pause for 100 milliseconds. In some embodiments, applying a pulsative flow comprises 100 to 400 pulses per milliliter of maternal sample and a repetitive flow pattern comprising an increase in flow rate to a flow rate in a range of 1-5 μl/sec for 100-200 milliseconds and a pause for a period of time in a range from 100 milliseconds to 1 second.

In some embodiments, the binding moieties are anti-EpCAM antibodies. In some embodiments, the method further comprises identifying the at least one multinucleated fetal giant cell by staining with anti-EpCAM antibodies, or by staining the nuclei or DNA with a Hoechst stain. In some embodiments, the method further comprises identifying the at least one multinucleated fetal giant cell by FISH probe signals. In some embodiments, the sample is maternal whole blood admixed with a whole blood buffer composition comprising a ficoll polysaccharide and a halogenated sucrose derivative.

In some embodiments, the sample is maternal whole blood admixed with a dual whole blood and fixative buffer composition comprising a ficoll polysaccharide, a halogenated sucrose derivative, an anticoagulant, at least one of glyoxal or paraformaldehyde or formaldehyde or a formal acetic acid or a formal saline or a phosphate formalin or a formalin calcium, and a pH buffer.

In some embodiments, the binding moieties are immobilized by linkers. In some embodiments, the method further comprises degrading the linkers and recovering the fetal cells in an eluate. In some embodiments, the method further comprises lysing the recovered fetal cells in the eluate to obtain fetal nucleic acids.

In some embodiments, the method further comprises lysing isolated cells and recovering fetal nucleic acids. In some embodiments, the method further comprises recovering isolated fetal cells or fetal DNA in an eluate; providing one or more microfluidic chips for detection or analysis of fetal cells or fetal DNA, wherein each microfluidic chip comprises an inlet port, an outlet port, and microchannels in fluid communication with the inlet port and outlet port; providing a pair of automated pipettes corresponding to each microfluidic chip, comprising a first automated pipette comprising a first pump, and a first pipette tip containing the eluate or a solution comprising the isolated fetal cells or fetal DNA and coupled to the inlet port; a second automated pipette comprising a second pump, and a second pipette tip coupled to the outlet port; and wherein the controller is further programmed to command the first pump of the first automated pipette and the second pump of the second automated pipette to control flow of the eluate or the solution through the microfluidic chip; and hydrodynamically processing the eluate or the solution through said one or more microfluidic chips. In some embodiments, recovery of the fetal cells occurs in less than 2 hours.

In some embodiments, the method further comprises using the fetal cells from the eluate for cell-based analysis. In some embodiments, the cell-based analysis is ICC, or fluorescent in situ hybridization (FISH). In some embodiments, the method further comprises using the fetal nucleic acids for nucleic acid based analysis. In some embodiments, the nucleic acid based analysis is qPCR, NGS, amplification, DNA sequencing, or RNA sequencing, northern blotting, southern blotting, or microarray analysis. In some embodiments, the method further comprises using the fetal cells from the eluate to detect autosomal and sex chromosome aneuploidies, microdeletions, or duplications. In some embodiments, the method further comprises using the fetal nucleic acids to detect a single gene disorder.

The present disclosure describes a whole blood buffer composition and related methods for blood collections. The whole blood buffer compositions described herein not only provide stabilization of multiple types of analytes (cells, exosomes, cfDNA) in whole blood samples, but also enables use of whole blood samples for microfluidic applications by eliminating microclotting of whole blood samples in microfluidic devices (e.g. chips with micron size channels).

### BRIEF DESCRIPTION OF THE DRAWINGS

The accompanying drawings, which are incorporated herein and form part of the specification, illustrate various embodiments of the present invention and, together with the description, further serve to explain the principles of the invention and to enable a person skilled in the pertinent art to make and use the invention. In the drawings, like reference numbers indicate identical or functionally similar elements.

FIG. 1a is a schematic diagram of an example fluid-tight flow system according to embodiments of the present disclosure, and additional components of real-time feedback control according to embodiments of the present disclosure.

FIG. 1B illustrates an example fluid-tight flow system including a controller a pipetting instrument, e.g. an automated liquid handler, comprising multiple automated pipettes, multiple microfluidic chips each with an inlet port and outlet port, and an instrument deck to support the microfluidic chip, pipette tips, samples, reagents, workstations for sample processing.

FIG. 1c is a perspective view of multiple pipettes and microfluidic chips, wherein the pipette tips are coupled to the inlet port and outlet port of the respective microfluidic chip, according to embodiments of the present disclosure.

FIG. 2a, on the left, is a perspective view of the pipettes and microfluidic chip, wherein the pipette tips are coupled to the inlet port and outlet port of the microfluidic chip, respectively, according to embodiments of the present disclosure; and on the right, is a vertical sectional view of the same according to embodiments of the present disclosure.

FIG. 2b is a cross sectional view of the pipette tips coupled to the inlet port and outlet port of the microfluidic chip, respectively, according to embodiments of the present disclosure.

FIG. 2c is an exploded view of the microfluidic chip showing the base plate and cover plate (center), a perspective view of the assembled microfluidic chip (right), and a magnified view of channel recesses of the base plate (inset), according to embodiments of the present disclosure.

FIG. 2d is an image of the base plate (top, left), the cover plate (bottom, left), and the assembled microfluidic chip (right).

FIG. 2e is a cross-section of the microfluidic chip (taken on the plane indicated in FIG. 2c where the base plate and cover plate meet) with a top view of the base plate of the microfluidic chip according to embodiments of the present disclosure (cover plate not shown), according to embodiments of the present disclosure.

FIG. 2f (top left) is an image of microfluidic chip with an inlet port, outlet port, and connected multiple parallel channels with a sinusoidal shape, and (top right) multiple magnified images of sinusoidal channels. FIG. 2f (bottom) is a 100× magnified image of a cross-section of two channels of the microfluidic chip, according to embodiments of the present disclosure.

FIG. 3 is the backend software architecture for preparing firmware commands of a Hamilton Microlab STAR line liquid handler according to one embodiment of the present disclosure.

FIG. 4a is a flow chart including exemplary methods according to embodiments of the present invention.

FIG. 4b is an exemplary schematic of coordinating commands and firmware parameters to control z-drive motors and pipetting drive motors of pipettes 1 and 2 according to embodiments of the present invention.

FIG. 4c is a flow chart including exemplary methods according to embodiments of the present invention.

FIG. 4d is a flow chart including exemplary methods for conducting analysis on the data from the pressure sensors according to embodiments of the present invention.

FIG. 4e is a chart of exemplary real-time feedback control parameters to avoid over-pressure in a microfluidic chip according to embodiments of the present invention.

FIG. 4f is a flow chart including exemplary methods for conducting analysis on the data from the pressure sensors according to embodiments of the present invention.

FIG. 5a are images from the fluorescence microscope of a) a maternal cell showing FISH probe signals on two X chromosomes, b) a fetal cell showing FISH probe signals on three X chromosomes, c) a fetal cell showing FISH probe signals (3 each) for chromosome 21 and chromosome 13, and d) a fetal cell showing FISH probe signals (3 each) for chromosome 21 and chromosome 18.

FIG. 5b are images from the fluorescence microscope of a) a multinucleated fetal giant cell showing FISH probe signals (multiples of each) for chromosome 21 and chromosome 13, and b) a multinucleated fetal giant cell showing FISH prove signals (multiples of each) for chromosome 21 and chromosome 18.

FIG. 6a are images from the fluorescence microscope of a fetal cell from a patient sample that was positive for CK7, negative for CD45.

FIG. 6b are images from the fluorescence microscope of fetal cells from a patient sample—two were positive for CK45, of which one was positive for CD7.

FIG. 7 is a graph showing qPCR data on patient sample PN01295.

FIG. 8 shows full chip images of a chip that utilized the BioFluidica blood buffer (A) and a chip that utilized the Patent EP 315091B 1 Formulation #74 buffer (B).

FIG. 9 shows a full chip image of a chip that utilized the BioFluidica blood buffer (A), an enlarged image of the inlet channel and entry of the sinusoidal microchannels (B, top) and the outlet channel and exit of the sinusoidal microchannels (B, bottom), and a further enlarged image of the inlet channel and entry of the sinusoidal microchannels (C).

FIG. 10 shows a full chip image of a chip that utilized the Patent EP 315091B I Formulation #74 blood buffer (A), an enlarged image of the inlet channel and entry of the sinusoidal microchannels (B, top) and the outlet channel and exit of the sinusoidal microchannels (B, bottom), and a further enlarged image of the inlet channel and entry of the sinusoidal microchannels (C).

### BRIEF DESCRIPTION OF THE SEQUENCE LISTING

SEQ ID NO: 1 represents an oligonucleotide linker.

SEQ ID NO: 2 represents an oligonucleotide linker.

SEQ ID NO: 3 represents an oligonucleotide linker.

SEQ ID NO: 4 represents an oligonucleotide linker.

### DETAILED DESCRIPTION OF EMBODIMENTS

While the present invention may be embodied in many different forms, a number of illustrative embodiments are described herein with the understanding that the present disclosure is to be considered as providing examples of the principles of the invention and such examples are not intended to limit the invention to preferred embodiments described herein and/or illustrated herein. The claimed subject matter might also be embodied in other ways, to include different steps or elements similar to the ones described in this document, in conjunction with other present or future technologies. Moreover, although the term “step” may be used herein to connote different aspects of methods employed, the term should not be interpreted as implying any particular order among or between various steps herein disclosed unless and except when the order of individual steps is explicitly described.

Embodiments of the present invention will now be described more fully hereinafter with reference to the accompanying drawings, in which some, but not all, embodiments of the invention are shown. Indeed, the invention may be embodied in many different forms and should not be construed as limited to the embodiments set forth herein; rather, these embodiments are provided so that this disclosure will satisfy applicable legal requirements. Like numbers refer to elements throughout. Other details of the embodiments of the invention should be readily apparent to one skilled in the art from the drawings. Although the invention has been described based upon these preferred embodiments, it would be apparent to those skilled in the art that certain modifications, variations, and alternative constructions would be apparent, while remaining within the spirit and scope of the invention.

It is to be understood that all ranges described herein comprise all subranges therein. As illustration, the range “from 50 to 80” includes all possible ranges therein (e.g., 51-79, 52-78, 53-77, 54-76, 55-75, 60-70, etc.). Furthermore, all values within a given range include the endpoint of a range (e.g., the range 50-80 includes the ranges with endpoints such as 55-80, 50-75, etc.).

The presently disclosed subject matter is now described in more detail.

The inventions disclosed herein relate to non-invasive prenatal screening, including screening to identify genetic abnormalities and inform prenatal and perinatal care. The inventions disclosed herein are methods of isolating fetal cells, such as trophoblasts and multinucleated fetal giant cells, from a maternal whole blood sample from a pregnant subject, that can be used for screening or diagnosis. The inventions disclosed herein also relate to whole blood buffer compositions and related methods for eliminating microclotting and aggregation for microfluidic applications.

Definitions

“Fetal cell” means a cell derived from the fetus. “Microchannel” means a channel having dimensions of less than 1 mm.

“Pipette” means a pipetting structure that can aspirate and/or dispense a liquid into or from a pipette tip. Liquid handling systems often refer to pipettes as pipetting channels.

“Binding moiety” means a chemical species to which a cell binds. Exemplary binding moieties include antibodies, aptamers (nucleic acid based high-affinity ligands that bind to antigens), high affinity peptides, affimers, haptens, nucleic acids, proteins, synthetic polymers, or carbohydrates.

“Selectively binds” refers to binding specificity and the ability of a binding moiety to discriminate between similar and dissimilar antigens.

“Enrichment of fetal cells” means the ratio of fetal cells to maternal cells of the sample is increased. The fold of enrichment is preferably more than 100 fold, more than 300 fold, more than 500 fold, more than 1000 fold, even more preferably more than 10,000 fold and most preferably more than 100,000 fold. As described in some embodiments herein, fold of enrichment can be achieved with a given binding moiety or combination of binding moieties. The fold enrichment can be calculated by dividing the number of target cells recovered by the total number of non-target cells in the starting sample.

“Bi-directionally loading a sample” means hydrodynamically processing a sample through the microfluidic chip, aspirating the sample into the second pipette tip, reversing the direction of flow and hydrodynamically processing the sample through the microfluidic chip in the reverse direction. In some embodiments, the method comprises bi-directionally loading a sample multiple times.

“Isolate” means capture or immobilize. As described herein, a fetal cell is isolated wherein the fetal cell contacts a binding moiety. The number of isolated fetal cells is at least the number of recovered fetal cells.

“Recover” means obtaining (for example, in an eluate) after release of isolated cells (for example, after enzymatic cleavage of a linker).

“Purity” means the percent representation of target fetal cells within a background of non-targeted cells. For example, 80% purity means 80% of the cells isolated and recovered are target fetal cells, e.g. the percentage of particular antibody/antibody combination positive cells among recovered cells. As described in some embodiments herein, increased purity can be achieved with a given binding moiety or combination of binding moieties. Purity can be calculated by dividing the number of fetal cells isolated and recovered in the eluate by the number of non-fetal cells in the eluate.

“About” as used herein when referring to a measurable value such as an amount, a temporal duration, and the like, is meant to encompass variations of ±5%, or even ±1% from the specified value, as such variations are appropriate for the disclosed compositions or to perform the disclosed methods.

“Surface marker” means a protein expressed on the surface of a cell.

“Multinucleated fetal giant cells” herein refers to multinucleated cells (such as cells formed from fusion of cytotrophoblasts, or fusion of extravillous trophoblasts), and multinucleated vesicles (such as syncytial nuclear aggregates shed from the syncytiotrophoblast, which is a single multinucleated fetal cell covering the human placenta) of fetal origin. Nuclei are not always distinct and nuclear content may appear as diffuse throughout the cell or vesicle structure.

“Pulsative flow” is a hydrodynamic profile which requires that fluid move through a microchannel or conduit in a pulsating fashion transitioning between high and low pressures.

“Channel width” and “channel height” are dimensions of a cross-section of the microchannel, each measured in a plane perpendicular to the plane in which the microchannels extend from the inlet port to the outlet port. The channel width is perpendicular to the channel height, and the channel width is the smaller of the two dimensions.

“Eluate” means a solution obtained at least in part by elution. Elution is the process of extracting a substance (e.g., cells or DNA) by washing it with a liquid (e.g., a buffer).

Methods of Isolating Fetal Cells from a Maternal Whole Blood Sample from a Pregnant Subject

Described herein are exemplary embodiments of a method of isolating fetal cells from a sample from a pregnant subject. In an embodiment, the method of isolating fetal cells from a sample from a pregnant subject comprises providing an isolation system comprising a microfluidic chip comprising an inlet port, an outlet port, and multiple parallel sinusoidal microchannels in fluid communication with the inlet port and outlet port. The sinusoidal microchannels comprise at least one of: binding moieties that selectively bind to EpCAM, binding moieties that selectively bind to CD105, and/or binding moieties that selectively bind to CD141. For example, the sinusoidal microchannels can comprise binding moieties that selectively bind to EpCAM, alone or in combination with binding moieties that selectively bind to CD105, and/or binding moieties that selectively bind to CD141. For example, the sinusoidal microchannels can comprise binding moieties that selectively bind to CD105, alone or in combination with binding moieties that selectively bind to EpCAM, and/or binding moieties that selectively bind to CD141. For example, the sinusoidal microchannels can comprise binding moieties that selectively bind to CD141, alone or in combination with binding moieties that selectively bind to CD105, and/or binding moieties that selectively bind to CD105.

The method of isolating fetal cells from a sample from a pregnant subject further comprises providing a liquid handling system comprising a pair of automated pipettes corresponding to each microfluidic chip: a first automated pipette comprises a first pump, and a first pipette tip containing a sample from a pregnant subject and coupled to the inlet port; and a second automated pipette comprises a second pump, and a second pipette tip coupled to the outlet port. The liquid handling system further comprises a controller comprising a non-transitory computer readable medium in communication with the first pump and the second pump, and programmed to command the first pump of the first automated pipette and the second pump of the second automated pipette to control flow of the sample through the microfluidic chip. The first pipette tip contains a sample from a pregnant subject, wherein the sample is maternal whole blood, has not been processed to remove maternal cells, and comprises at least one fetal cell. Generally, there is a high degree of heterogeneity among samples from the same pregnant subject (e.g. depending on the gestation week a sample is taken, how the sample is obtained, etc.) and a high degree of heterogeneity among samples from a population of pregnant subjects. Accordingly, the first pipette tip contains a sample from a pregnant subject that comprises at least one fetal cell and the methods disclosed herein describe methods of isolating fetal cells from a sample from a pregnant subject when at least one fetal cell is present for isolation.

The method of isolating fetal cells from a sample from a pregnant subject further comprises hydrodynamically processing the sample through said one or more microfluidic chips. The method of isolating fetal cells from a sample from a pregnant subject further comprises isolating at least one fetal cell, wherein the at least one fetal cell contacts at least one of said binding moieties, and wherein the method does not comprise removing maternal cells prior to isolation of the at least one fetal cell. In some embodiments, the method comprises recovering at least one fetal cell in the presence of at least 500 maternal cells, at least 750 maternal cells, or at least 1000 maternal cells. In some embodiments, fetal cell purity is greater than 5%, 10%, 15%, or 20% of the population of cells isolated and recovered.

The methods described herein advantageously and unexpectedly isolate fetal cells from whole blood using binding moieties that selectively bind to EpCAM, binding moieties that selectively bind to CD105, and/or binding moieties that selectively bind to CD141 and without sample processing steps or isolation steps that remove maternal cells.

The number of fetal cells in maternal circulation can be in the range of 1 nucleated cell per 10⁴ to 1 per 10⁹ nucleated maternal cells. (Krabchi et al., Quantification of all fetal nucleated cells in maternal blood between the 18th and 22nd weeks of pregnancy using molecular cytogenetic techniques. Clin Genet 2001;60(2):145-50) Krabchi et al. measured the absolute number of all different types of male fetal nucleated cells per unit of volume of peripheral maternal blood obtained between 18 and 22 weeks of pregnancy, and identified 2 to 6 male fetal nucleated cells per milliliter of maternal blood by identifying fetal XY cells among maternal XX cells by fluorescence in situ hybridization (FISH) and primed in situ labeling (PRINS), without enrichment, along with manual examination of cells. Others have reported one fetal cell per 10⁵ -10⁸ nucleated maternal cells or 1-10 fetal cells per ml maternal blood.

At least for the past decade, methods of isolating fetal cells from whole blood have generally included enrichment protocols that involve initial blood processing steps to remove excess maternal cells such as: selective density gradient centrifugation (DGC) to separate out maternal red blood cells and plasma, selective lysis of maternal red blood cells, and magnetic-activated cell sorting (MACS) utilizing maternal white blood cell markers to deplete maternal white blood cells.

In particular, use of anti-CD105, anti-CD141, and anti-EpCAM antibodies to isolate fetal cells has required enrichment steps to remove maternal cells from whole blood in order to be able to isolate fetal cells. The selectivity of the anti-CD105, anti-CD141, and anti-EpCAM antibodies has been dependent on removal of maternal cells from whole blood, a reduction of 1.3×10¹¹ RBCs in some instances.

In 2014, Hatt et al. (Fetal Diagn Ther. 2014; 35(3):218-27) describes identification and enrichment of fetal cells from maternal blood. Using magnetic cell sorting (MACS) enrichment, Hatt et al. reports identification of 3.2 fetal cells per 10 ml of blood (with CD105), 0.36 fetal cells per 10 ml of blood (with CD34), 0.56 fetal cells per 10 ml of blood (with CD141), and 0 fetal cells per 10 ml of blood (with CD146). Each blood sample was processed to stabilize or fix cells with formaldehyde and to remove maternal cells. Maternal erythrocytes were lysed with a lysis buffer comprises a non-ionic detergent, preferably Triton X-100, and unlysed cells are pelleted by centrifugation. Thus, even with steps to initially deplete maternal cells, the best results that could be obtained was 0.32 fetal cells per ml of blood with CD105 and 0.06 fetal cells per ml of blood with CD141. (see also FCMB APS US Patent Publication 20120003643, wherein each example relies on the same maternal blood processing steps (Example 1) involving fixation with formaldehyde, and lysis of maternal erythrocytes and separation by centrifugation.)

Hatt et al. also reported an average of 4.3 fetal cells per 30 ml of blood (or 0.14 fetal cells per ml) using the same blood processing methods to fix cells and remove maternal cells and MACS enrichment with antibodies for CD105 and CD141 (Prenatal Diagnosis 2014, 34, 1066-1072). Hatt et al. recognized that prior attempts failed to isolate fetal cells using the marker CD105 without the fixation step; thus, suggesting that the fixation step is necessary. Hatt et al. further recognized that there was relatively large cell loss through the enrichment procedure. Hatt et al. were unable to achieve their ultimate goal of find enough fetal cells to generate representative genomic DNA from the fetus through whole genome amplification.

In 2016, Arcedi Biotech Aps reported an average of 12.8 fetal cells from 30 ml of maternal blood (or 0.43 fetal cells per ml) using the same blood processing methods to fix cells and remove maternal cells and MACs enrichment with antibodies for CD105 (as described in U.S. Pat. No. 9,429,520) (Kølvraa et al., Prenatal Diagnosis 2016, 36, 1127-1134, see also U.S. Pat. No. 9,429,520). The blood processing step of fixation and RBC lysis reduced the approximate number of maternal cells by 1.3×10¹¹ RBCs.

Fast forward almost four years, and Arcedi continues to rely on the following steps for isolation of rare fetal cells: 1) removal of erythrocytes, e.g. by lysis of erythrocytes, 2) magnetic bead enrichment with microbeads labeled with antibodies against endothelial/mesenchymal cell surface markers, e.g. CD105, 3) staining of fetal cells with anti-cytokeratin antibodies, and 4) identifying fetal cells on slides with an automated fluorescence microscope scanner. (Ravn et al. The Number of Circulating Fetal Extravillous Trophoblasts Varies from Gestational Week 6 to 20. Reprod. Sci. (2020)). Ravn et al. reported an average of 5.5 fetal extravillous trophoblasts (fEVTs) per 30 ml of blood with blood samples drawn at gestational week (GW) 6-8, an average of 11.9 fEVTs per 30 ml of blood with blood samples drawn at GW 12-13, and an average of 5.3 fEVTs per 30 ml of blood with blood samples drawn at GW 19-20. Thus, the highest reported average was 11.9 fEVTs per 30 ml (or 0.40 fetal cells per ml) of blood with blood samples drawn at GW 12-13.

In 2017, Hou et al. described enrichment of whole blood samples by gradient centrifugation to deplete maternal red blood cells and separate out a peripheral blood mononuclear cell layer containing circulating trophoblasts, prior to cell capture using imprinted NanoVelcro Microchips with anti-EpCAM antibodies. (Hou et al. Hou S, Chen J-F, Song M, Zhu Y, Jan Y J, Chen S H, et al. Imprinted NanoVelcro microchips for isolation and characterization of circulating fetal trophoblasts (cTBs): toward noninvasive prenatal diagnostics. ACS Nano 2017; 11(8):8167-77). Hou et al. reported 3-6 cTBs on the imprinted nanoVelcro substrates from 2 mL of these maternal blood samples for the healthy cohort, and 5-15 cTBs per 2 mL of maternal blood for the diseased cohort. Thus, teachings regarding successful use of anti-CD105, anti-CD141, and anti-EpCAM antibodies for fetal cell isolation are tied to teachings of initial blood processing steps to remove maternal cells.

The methods of isolating fetal cells described herein without removal of maternal cells are contrary to accepted wisdom in the art. Moreover, given the selectivity of the anti-CD105, anti-CD141, and anti-EpCAM antibodies for fetal cell isolation has been dependent on an initial removal of maternal cells from whole blood, it is unexpected that the methods described herein could isolate fetal cells from whole blood using binding moieties that selectively bind to EpCAM, binding moieties that selectively bind to CD105, and/or binding moieties that selectively bind to CD141 without sample processing steps or isolation steps that remove maternal cells. It is further surprising that fetal cell isolation results described herein are on par with or better than fetal cell isolation results obtained from known methods with an initial step of depleting maternal cells using antibodies against the same fetal cell surface markers. It is further surprising to recover at least one fetal cell in the presence of at least 500 maternal cells, at least 750 maternal cells, or at least 1000 maternal cells. It is further surprising that an eluate comprising at least one fetal cell in the presence of at least 500 maternal cells, at least 750 maternal cells, or at least 1000 maternal cells yielded successful downstream testing results, e.g. successful FISH results.

In some embodiments, the isolation system comprises two or more microfluidic chips, wherein each first pipette tip of each first automated pipette of each pair of automate pipettes corresponding to each chip comprises the same sample, wherein hydrodynamically processing the sample through said two or more microfluidic chips comprises flowing a portion of the same sample through each microfluidic chip, and the method comprises isolating an average of 0.5 fetal cells per milliliter of sample. In some embodiments, the isolation system comprises eight microfluidic chips and eight pairs of automated pipettes with each respective first pipette tip containing at least 1 milliliter of the same sample, and wherein hydrodynamically processing the sample through said eight microfluidic chips comprises flowing the at least lml of sample in each first pipette tip through the corresponding microfluidic chip. In some embodiments, the sample is diluted 20:1, 19:1, 18:1, 17:1, 16:1, 15:1, 14:1, 13:1, 12:1, 11:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19, 1:20 in 1×PBS prior to flowing through the microfluidic chip.

The presently disclosed methods of isolating fetal cells also describe certain synergistic combinations of binding moieties that, together with the microfluidic chip and the liquid handling system, have a synergistic effect.

In some embodiments, the microchannels comprise a synergistic combination of binding moieties that selectively bind to EpCAM and binding moieties that selectively bind to CD141. The binding moieties that selectively bind to EpCAM and the binding moieties that selectively bind to CD141, when used with the microfluidic chip described herein and the liquid handling system described herein, are more effective in isolating fetal cells when in combination than when applied individually. In some embodiments, the method comprises isolating at least 3 fetal cells per milliliter of sample. In some embodiments, the method comprises recovering at least three fetal cells in the presence of at least 500 maternal cells, at least 750 maternal cells, or at least 1000 maternal cells. In some embodiments, fetal cell purity is greater than 5%, 10%, 15%, or 20% of the population of cells isolated and recovered. In some embodiments, the isolation system comprises two or more microfluidic chips, wherein each first pipette tip of each first automated pipette of each pair of automate pipettes corresponding to each chip comprises the same sample, wherein hydrodynamically processing the sample through said two or more microfluidic chips comprises flowing a portion of the same sample through each microfluidic chip, and the method comprises isolating at least an average of 3 fetal cells per milliliter of sample. In some embodiments, the method comprises recovering at least three fetal cells in the presence of at least 500 maternal cells, at least 750 maternal cells, or at least 1000 maternal cells. In some embodiments, fetal cell purity is greater than 5%, 10%, 15%, or 20% of the population of cells isolated and recovered.

In some embodiments, the method comprises microchannels comprise a synergistic combination of binding moieties that selectively bind to EpCAM and binding moieties that selectively bind to CD105. The binding moieties that selectively bind to EpCAM in combination with the binding moieties that selectively bind to CD105, when used with the microfluidic chip described herein and the liquid handling system described herein, are more effective in isolating fetal cells when in combination than when applied individually. In some embodiments, the method comprises isolating at least 2 fetal cells per milliliter of sample. In some embodiments, the method comprises recovering at least two fetal cells in the presence of at least 500 maternal cells, at least 750 maternal cells, or at least 1000 maternal cells. In some embodiments, fetal cell purity is greater than 5%, 10%, 15%, or 20% of the population of cells isolated and recovered. In some embodiments, the isolation system comprises two or more microfluidic chips, wherein each first pipette tip of each first automated pipette of each pair of automate pipettes corresponding to each chip comprises the same sample, wherein hydrodynamically processing the sample through said two or more microfluidic chips comprises flowing a portion of the same sample through each microfluidic chip, and the method comprises isolating at least an average of 2 fetal cells per milliliter of sample. In some embodiments, the method comprises recovering at least two fetal cells in the presence of at least 500 maternal cells, at least 750 maternal cells, or at least 1000 maternal cells. In some embodiments, fetal cell purity is greater than 5%, 10%, 15%, or 20% of the population of cells isolated and recovered.

In some embodiments, the fetal cells are fetal trophoblastic cells. In some embodiments, the fetal cells are extravillous trophoblast cells. In some embodiments, the sample from a pregnant subject is taken during gestation week 8, 9, 10, 11, 12, 13, 14, 15, 16, 17, 18, 19, 20, 21, 22, 23, 24, 25, 26, 27, 28, 29, 30, 31, 32, 33, 34, or 35.

In some embodiments, the sample is maternal whole blood admixed with a whole blood buffer composition comprising a ficoll polysaccharide and a halogenated sucrose derivative. In some embodiments, the whole blood buffer composition further comprises amino acids, human serum proteins and a Bax channel blocker. In some embodiments, the sample is maternal whole blood admixed with a dual whole blood and fixative buffer composition comprising a ficoll polysaccharide, a halogenated sucrose derivative, an anticoagulant, at least one of glyoxal or paraformaldehyde or formaldehyde or a formal acetic acid or a formal saline or a phosphate formalin or a formalin calcium, and a pH buffer. In some embodiments, the method comprises bi-directionally loading a sample.

In some embodiments, formation of microclots in the microfluidic chip is inhibited. In some embodiments, the flow rate of the admixed whole blood through the microchannels on the chip is up to 25 μl/min without clogging. In some embodiments, maternal cell lysis and release of maternal cellular nucleic acids is inhibited. In some embodiments, the method comprises stabilizing nucleated cells in whole blood at ambient temperatures. In some embodiments, the method comprises inhibiting lysis of nucleated cells and release of cellular nucleic acids.

In an embodiment, hydrodynamically processing the sample through said one or more microfluidic chips comprises applying a pulsative flow. In an embodiment, the overall flow rate is in the range of 50 to 300 per minute, 100 to 250 per minute, 100 to300 per minute, 50 to 150 per minute, 50 to 100 per minute, or 100 to 150 per minute.

Described herein are also exemplary embodiments of a method of isolating multinucleated fetal giant cells from a sample from a pregnant subject. In an embodiment, the method of isolating multinucleated fetal giant cells from a sample from a pregnant subject comprises providing an isolation system comprising one or more microfluidic chips, wherein each microfluidic chip comprises an inlet port, an outlet port, and multiple parallel sinusoidal microchannels in fluid communication with the inlet port and outlet port. The sinusoidal microchannels have a channel width in the range of about 15 μm to about 45 μm and a channel height in the range of about 100 μm to about 150 μm, and the sinusoidal microchannels comprise binding moieties that selectively bind to a surface marker of fetal cells. The method of isolating multinucleated fetal giant cells from a sample from a pregnant subject further comprises providing a liquid handling system comprising a pair of automated pipettes corresponding to each microfluidic chip: a first automated pipette comprising a first pump, and a first pipette tip containing a sample from a pregnant subject and coupled to the inlet port, wherein the sample is maternal whole blood and; a second automated pipette comprising a second pump, and a second pipette tip coupled to the outlet port. The liquid handling system further comprises a controller comprising a non-transitory computer readable medium in communication with the first pump and the second pump, and programmed to command the first pump of the first automated pipette and the second pump of the second automated pipette to control flow of the sample through said one or more microfluidic chips. The method of isolating multinucleated fetal giant cells from a sample from a pregnant subject further comprises hydrodynamically processing the sample through said one or more microfluidic chips. In an embodiment, hydrodynamically processing the sample through said one or more microfluidic chips comprises applying a pulsative flow. The method of isolating multinucleated fetal giant cells from a sample from a pregnant subject further comprises isolating at least one multinucleated fetal giant cell, wherein the at least one multinucleated fetal giant cell contacts at least one of said binding moieties and wherein the at least one multinucleated fetal giant cell is 50 to 150 μm in diameter as measured along the longest axis. In some embodiments, the method comprises recovering at least one multinucleated fetal giant cell in the presence of at least 500 maternal cells, at least 750 maternal cells, or at least 1000 maternal cells.

Isolation of multinucleated fetal giant cells from whole blood was unexpected given the larger size of the multinucleated fetal giant cells (50 to 150 μm in diameter as measured along the longest axis) as compared to the smaller sinusoidal microchannel channel width (in the range of about 15 μm to about 45 μm and in particular a width of about 25 μm). The methods of isolating multinucleated fetal giant cells described herein also are advantageous due to access to larger DNA content per unit of capture.

In an embodiment, the sinusoidal microchannels have a channel width in the range of about 20 μm to about 25 μm and a channel height in the range of about 140 μm to about 150 m. In another embodiment, the sinusoidal microchannels have a channel width of about 20 μm and a channel height of about 150 μm.

In an embodiment, pulsative flow comprises a repetition of a pulse and pause. Pulsative flow can be achieved by a number of strategies such as the sequence pulse-pause with an impulse force in μL/second applied during the pulse phase and a full stop of defined duration during the pause phase. Another strategy is to continuously reduce and raise the impulse force without a formal pause.

In an embodiment, the method of isolating multinucleated fetal giant cells from a sample from a pregnant subject comprises applying a pulsative flow comprises 300 pulses per milliliter of maternal sample and a repetitive flow pattern comprising an increase in flow rate to 1 μl/sec for 100 milliseconds and a pause for 100 milliseconds. For example, the pulsative flow comprises 300 pulses per ml of blood sample following this pattern:
a. increase in flow rate to 1 μl/sec for 100 milliseconds;
b. pause for 100 milliseconds; and
c. repeat until blood sample is processed.
   The overall flow rate is 13 μl per minute.

In another embodiment, the method of isolating multinucleated fetal giant cells from a sample from a pregnant subject comprises applying a pulsative flow comprises 100 to 400 pulses per milliliter of maternal sample and a repetitive flow pattern comprising an increase in flow rate to a flow rate in a range of 1-5 μl/sec for 100-200 milliseconds and a pause for a period of time in a range from 100 milliseconds to 1 second. For example, the pulsative flow for the isolation of multinucleated fetal giant cells (ranging in size from about 15 μm to about 150 μm along the long axis) comprises 100-400 pulses per ml of blood sample following this pattern and ranges:
a. increase flow rate to a flow rate in the range of 1-5 μl/sec for 100-200 milliseconds;
b. pause for a period of time in the range from 100 milliseconds to 1 second; and
c. repeat until blood sample is processed.
   The overall flow rate is the range of 13 μl to 25 μl per minute. In an embodiment, the overall flow rate is in the range of 5 μl to 30 μl per minute, 10 μl to 25 μl per minute, 10 μl to 30 μl per minute, 5 μl to 15 μl per minute, 5 μl to 10 μl per minute, or 10 μl to 15 μl per minute.

The fluid-tight flow system described below allows for an extremely low flow rate by precise control of the pressure differential by coordination of the pipettes to drive the applied fluid through the chip. In one embodiment, a pulsative flow is applied by the pipettes to control flow of the blood sample through the microchannels. The pulse is an increase in flow rate caused by coordination of the pipettes (e.g. plunger of pipette 1 moving down to apply pressure and the plunger of pipette 2 moving up to create a vacuum). The pulse pushes the liquid sample through the channels. The pause is a decrease in flow rate caused by coordination of the pipettes (e.g. plunger of pipette stops and the plunger of pipette 2 stops). The pause causes turbulence and allows enhanced interaction of the cells with the interior surface of the microchannels and binding moieties. The pause also relieves pressure in the microchannels of the microchip that can cause lysis and backflow of the sample through the ports after the pipettes have disengaged.

The volume applied during a pulse can be calculated, for example, by dividing the applied volume of the liquid sample (e.g. one milliliter) by the number of pulses desired. For example, a one milliliter sample volume applied to a chip with 100 pulses would consist of pulse volumes of 10 μl each. The rate at which volume flows in microliters per second can range from about 1 μl to about 10 μl per second while the pause may be set for any length of time but is typically less than one second.

The pulsative flow slows down the overall flow rate of the sample through the microfluidic chip, beyond what is achievable by a constant flow rate. The extremely low flow rate provides multiple advantages. The pulsative flow breaks down laminar flow, thereby causing more cells to interact with the interior surface of the channels and increasing the roll rate, which in turn increases capture efficiency. This is particularly useful for isolating fetal cells with fewer receptors that bind to binding moieties.

In an embodiment, the sample from a pregnant subject has not been processed to remove maternal cells, and the method of isolating multinucleated fetal giant cells from a sample from a pregnant subject does not comprise removing maternal cells prior to isolation of the at least one fetal cell. In another embodiment, the sinusoidal microchannels comprise one or more binding moieties that selectively bind to a surface marker of fetal cells. In one embodiment, the sinusoidal microchannels comprise binding moieties that selectively bind to a first surface marker of fetal cells and binding moieties that selectively bind to a second surface marker of fetal cells. In one embodiment, the sinusoidal microchannels comprise binding moieties that selectively bind to EpCAM, binding moieties that selectively bind to CD105, and/or binding moieties that selectively bind to CD141.

The methods of isolating multinucleated fetal giant cells described herein without removal of maternal cells are contrary to accepted wisdom in the art. Moreover, given the selectivity of anti-EpCAM antibodies for fetal cell isolation and other binding moieties that selectively bind to a surface marker of fetal cells have been dependent on an initial removal of maternal cells from whole blood, it is unexpected that the methods described herein could isolate multinucleated fetal giant cells from whole blood using binding moieties that selectively bind to EpCAM, binding moieties that selectively bind to CD105, and/or binding moieties that selectively bind to CD141 without sample processing steps or isolation steps that remove maternal cells.

In some embodiments, the sample is maternal whole blood admixed with a whole blood buffer composition comprising a ficoll polysaccharide and a halogenated sucrose derivative. In some embodiments, the whole blood buffer composition further comprises amino acids, human serum proteins and a Bax channel blocker. In some embodiments, the sample is maternal whole blood admixed with a dual whole blood and fixative buffer composition comprising a ficoll polysaccharide, a halogenated sucrose derivative, an anticoagulant, at least one of glyoxal or paraformaldehyde or formaldehyde or a formal acetic acid or a formal saline or a phosphate formalin or a formalin calcium, and a pH buffer. In some embodiments, the method comprises bi-directionally loading a sample.

In some embodiments, formation of microclots in the microfluidic chip is inhibited. In some embodiments, the flow rate of the admixed whole blood through the microchannels on the chip is up to 25 μl/min without clogging. In some embodiments, maternal cell lysis and release of maternal cellular nucleic acids is inhibited. In some embodiments, the method comprises stabilizing nucleated cells in whole blood at ambient temperatures. In some embodiments, the method comprises inhibiting lysis of nucleated cells and release of cellular nucleic acids.

Whole Blood Sample and Whole Blood Sample Preparation

The maternal whole blood sample from a pregnant subject is preferably in the range of 0.5 to 50 ml, such as in the range of 1 to 40 ml, such as from 5 to 35 ml or 10 to 30 ml.

The maternal whole blood sample from a pregnant subject is preferably obtained from a pregnant woman between 8-18 weeks of gestation, more preferably between 8-10, or 16-18 weeks of gestation.

In an embodiment the sample from a pregnant subject is venous whole blood. In an embodiment, the sample was collected in a blood specimen collection tube. In another embodiment, the sample was collected in a blood specimen collection tube containing a solution of, but not limited to, EDTA, sucralose, PBS, or adenosine.

In another embodiment, the sample is maternal whole blood admixed with a whole blood buffer composition, or a dual whole blood and fixative buffer composition. In an embodiment, the sample is maternal whole blood admixed with a whole blood buffer composition in a ratio ranging from 1:5 to 1:10. In an embodiment, the sample is maternal whole blood admixed with a dual whole blood and fixative buffer composition in a ratio ranging from 1:5 to 1:10.

In vitro methods for analyte stabilization in a blood sample are also described herein. In an embodiment of an in vitro method for analyte stabilization in a blood sample, wherein the analyte is a cell, exosome, or cfDNA, the method comprises combining or admixing a sample of blood with a whole blood buffer composition. In an embodiment, the cells are fixed cells or non-fixed cells. In an embodiment, the cells are circulating tumor cells, circulating leukemic cells, trophoblasts, erythrocytes, or white blood cells

In an embodiment, the whole blood sample is admixed with a whole blood buffer composition in a ratio ranging from 1:5 to 1:10. In an embodiment, a whole blood sample is admixed with a whole blood buffer composition immediately after collection or blood draw. In another embodiment, a whole blood sample is admixed with a whole blood buffer composition during collection or blood draw (e.g. a blood collection tub comprises a whole blood buffer composition).

In some embodiments the whole blood sample is admixed with a whole blood buffer composition, or a dual whole blood and fixative buffer composition substantially contemporaneously from the time of blood draw (e.g., within less than about 10 minutes of the blood draw).

In an embodiment, the sample is a whole blood sample. In an embodiment, the whole blood sample is preferably in the range of 0.5 to 50 ml, such as in the range of 1 to 40 ml, such as from 5 to 35 ml or 10 to 30 ml. In an embodiment the sample from a subject is venous whole blood. In an embodiment, the sample was collected in a blood specimen collection tube. In another embodiment, the sample was collected in a blood specimen collection tube containing a solution of, but not limited to, EDTA, sucralose, PBS, or adenosine.

In some embodiments a lapse of time of at least about 2 hours, at least about 6 hours, at least about 24 hours, at least about 7 days, at least about 14 days, at least about 15 days, at least about 16 days, at least about 17 days, at least about 18 days, at least about 19 days, or at least about 20 days can elapse between the time of blood draw (which may be substantially contemporaneous with the admixing step), and the time of isolation of cells from the whole blood admixture. In some embodiments, the whole blood admixture is stored at ambient temperatures for at least 24 hours, at least 2 days, at least 3 days, or at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, or at least 20 days. In some embodiments, the nucleated cells are stabilized for at least 4 days, at least 5 days, at least 6 days, at least 7 days, at least 8 days, at least 9 days, at least 10 days, at least 11 days, at least 12 days, at least 13 days, at least 14 days, at least 15 days, at least 16 days, at least 17 days, at least 18 days, at least 19 days, or at least 20 days.

Described herein is a method of stabilizing analytes in a whole blood sample at ambient temperatures comprising admixing a whole blood sample ex vivo with a composition described herein. “Stabilizing” herein means providing stable storage of. In an embodiment, the analytes are cells (fetal cells, circulating tumor cells), cfDNA, or exosomes. In an embodiment, the analyte is stabilized in a collection tube. In an embodiment, the analyte is stabilized in a collection tube for at least six hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours or at least 24 hours.

Whole Blood Buffer Composition

In an embodiment, the whole blood buffer composition comprises a ficoll polysaccharide and a halogenated sucrose derivative. In an embodiment, the ficoll polysaccharide is present at 16-24% w/v (or 2.2-3.4 mM), and sucralose is present at 160-240 mM. In an embodiment, the ficollpolysaccharide is present at 1-10 mM, and sucralose is present at 160-240 mM. In an embodiment, the ficollpolysaccharide is present at 2-4 mM, and sucralose is present at 160-240 mM. The ficollpolysaccharide surprisingly mildly inhibits phase separation of the liquid components primarily through modifying rheological properties such as increasing the low-shear viscosity while the halogenated sucrose derivative interacts with the outer erythrocyte membrane causing it to expand and manifest spicule like structures to form an echinocyte morphology. The echinocyte halogenated sugar complex results in a membrane structure that is more electronegative and less dense relative to the surrounding media consisting of polymer saturated plasma, inducing the cells to float at neutral density and resist aggregation. In some embodiments, the whole blood buffer composition further comprises at least one of: a cell apoptosis inhibitor, a human serum albumin or a bovine serum albumin, peptides, purines, anticoagulant agent, and pH buffer; or a combination thereof. In some embodiments, the whole blood buffer composition further comprises amino acids, human serum proteins and a Bax channel blocker. In some embodiments, the whole blood buffer composition comprises the ficollpolysaccharide present at 16-24% w/v (or 2.2-3.4 mM) and the halogenated sucrose derivative present at 160-240 mM; and at least one of: alanyl-glutamine present at 160-240 mM, at least one of human serum albumin present at 1.6 to 2.4% w/v or the bovine serum albumin is present at 1.6 to 2.4% w/v, adenine present at 4.0-6.0 mM, at least one of ethylenediaminetetraacetic acid present at 90 to 110 mM or citrate present at 12.8 to 19.2 mM, a Bax channel inhibitor present at 0.08 to 0.12 mM, or at least one of a phospho-buffered saline present at 0.8 to 1.2× or a tris-buffered saline present at 0.8 to 1.2×, or a combination thereof. In some embodiments, the whole blood buffer composition comprises sucralose present at 5-10% w/v, Ficoll PM 70 is present at 15-25% w/v, Ala-gln dipeptide is present at 2-6% w/v, human serum albumin is present at 1-5% w/v, adenine is present at 1-2% w/v, a Bax channel inhibitor is present at 0.1-0.5% w/v, and phosphate buffered saline present at 51-76% w/v. In some embodiments, the whole blood buffer composition comprises sucralose present at 5-15% w/v, and at least one of Ala-Gln present at 0-10% w/v, human serum albumin present at 0-10% w/v, adenine present at 0-1% w/v, a Bax channel inhibitor present at 0-1% w/v, or phosphate buffered saline present at 60-95% w/v.

Advantageously, it has surprisingly been found that the whole blood buffer composition inhibits sedimentation of whole blood, induces cell buoyancy, inhibits cell aggreggation, and protects against cell damage. The ability to suspend blood in a homogeneous phase improves the preservation of whole blood and prevents inflammatory responses that can cause apoptosis, necrosis, or other deleterious effects on target cells. Further advantageously, it has surprisingly been found that the combination of a ficollpolysaccharide present at 16-24% w/v (or 2.2-3.4 mM) and a halogenated sucrose derivative present at 160-240 mM provides significantly improved inhibition of sedimentation of whole blood as compared to the ficollpolysaccharide alone and the halogenated sucrose derivative alone. ficollpolysaccharide present at 16-24% w/v is ficollpolysaccharide present at 2.2-3.4 mM. Further advantageously, the whole blood buffer composition inhibits formation of elongated echinocyte tube structures and cell membrane shedding. Advantageously, it has surprisingly been found that the dual whole blood buffer composition enables microfluidic isolation of cells without microclotting. The ability to maintain cells in a suspension facilitates the low pressure passage of whole blood through microfluidic devices and reduce masking of the target cell surface antigens with capture or sensing technology on the device substratum.

Exemplary formulations of the whole blood buffer composition include the formulations listed in the tables below.

TABLE 1

| Component | 10X Concentration | | Working Range | |
|---|---|---|---|---|
| Suraclose | 200 | mM | 20-40 | mM |
| Ficoll PM 70 | 20% | w/v | 2-4% | |
| Ala-Gln dipeptide | 200 | mM | 20-40 | mM |
| Human serum albumin (HSA) | 2% | w/v | 0.1-0.4% | |
| Adenine | 5.0 | mM | 0.5-1.0 | mM |
| EDTA | 100 | mM | 10 | mM |
| Bax Channel Inhibitor | 0.10 | mM | 0.01-0.02 | mM |

| | | |
|---|---|---|
| Phospho-Buffered-Saline, | 1X | 0.1-0.2X |
| pH 7.2 | | |

TABLE 2

| Component | 10X Concentration | | Working Range | |
|---|---|---|---|---|
| Suraclose | 200 | mM | 20-40 | mM |
| Ficoll PM 70 | 20% | w/v | 2-4% | |
| Ala-Gln dipeptide | 200 | mM | 20-40 | mM |
| BSA (Fraction VIII) | 2% | w/v | 0.2-0.4% | |
| Adenine | 5.0 | mM | 0.5-1.0 | mM |
| Citrate | 16 | mM | 1.6-3.2 | mM |
| Bax Channel Inhibitor | 0.10 | mM | 0.01-0.02 | mM |

| | | |
|---|---|---|
| Tris Buffered Saline, pH 7.2 | 1X | 0.1-0.2X |

In an exemplary embodiment, a dual whole blood and fixative buffer composition a ficollpolysaccharide, a halogenated sucrose derivative, and at least one of glyoxal or paraformaldehyde or formaldehyde or a formal acetic acid or a formal saline or a phosphate formalin or a formalin calcium. In some embodiments, the dual whole blood and fixative buffer composition further comprises an anticoagulant. In some embodiments, the dual whole blood and fixative buffer composition further comprises a pH buffer. In some embodiments, the ficollpolysaccharide is present at 8-12% w/v, and the halogenated sucrose derivative is present at 160-240 mM. In some embodiments, the anticoagulant is citrate present at 12.8 to 19.2 mM. In some embodiments, at least one of: glyoxal is present at 2.4 to 3.6% or paraformaldehyde is present at 3.2% to 4.8% or formaldehyde is present at 1.6 to 2.4% or the formal acetic acid is present at 1.6 to 2.4% or the formal saline is present at 1.6 to 2.4% or the phosphate formalin is present at 1.6 to 2.4% or the formalin calcium is present at 1.6 to 2.4%. In some embodiments, the pH buffer is present at 0.8 to 1.2×.

Advantageously, it has surprisingly been found that the dual whole blood and fixative buffer composition stabilizes nucleated blood cells by inhibiting cell lysis. Exemplary formulations of dual whole blood and fixative buffer compositions include the formulations listed in the tables below.

TABLE 3

| Component | 10X Concentration | | Working Range | |
|---|---|---|---|---|
| Suraclose | 200 | mM | 20-40 | mM |
| Ficoll PM 70 | 10% | w/v | 1-2% | |
| Citrate | 16 | mM | 1.6-3.2 | mM |

| | | |
|---|---|---|
| Glyoxal | 3.0% | 0.3-0.6% |
| Tris-Buffered-Saline, pH 6.8 | 1X | 0.1-0.2X |

TABLE 4

| Component | 10X Concentration | | Working Range | |
|---|---|---|---|---|
| Suraclose | 200 | mM | 20-40 | mM |
| Ficoll PM 70 | 10% | w/v | 1-2% | |
| Citrate | 16 | mM | 1.6-3.2 | mM |

| | | |
|---|---|---|
| Paraformaldehyde | 4.0% | 0.4-0.8% |
| Tris-Buffered-Saline, pH 6.8 | 1X | 0.1-0.2X |

TABLE 5

| Component | 10X Concentration | | Working Range | |
|---|---|---|---|---|
| Suraclose | 200 | mM | 20-40 | mM |
| Ficoll PM 70 | 10% | w/v | 1-2% | |
| Citrate | 16 | mM | 1.6-3.2 | mM |

| | | |
|---|---|---|
| Formaldehyde | 2.0% | 0.2-0.4% |
| Tris-Buffered-Saline, pH 6.8 | 1X | 0.1-0.2X |

TABLE 6

| Component | 10X Concentration | | Working Range | |
|---|---|---|---|---|
| Suraclose | 200 | mM | 20-40 | mM |
| Ficoll PM 70 | 10% | w/v | 1-2% | |
| Citrate | 16 | mM | 1.6-3.2 | mM |

| | | |
|---|---|---|
| Formal acetic acid | 2.0% | 0.2-0.4% |
| Tris-Buffered-Saline, pH 6.8 | 1X | 0.1-0.2X |

TABLE 7

| Component | 10X Concentration | | Working Range | |
|---|---|---|---|---|
| Suraclose | 200 | mM | 20-40 | mM |
| Ficoll PM 70 | 10% | w/v | 1-2% | |
| Citrate | 16 | mM | 1.6-3.2 | mM |

| | | |
|---|---|---|
| Formal Saline | 2.0% | 0.2-0.4% |
| Tris-Buffered-Saline, pH 6.8 | 1X | 0.1-0.2X |

TABLE 8

| Component | 10X Concentration | | Working Range | |
|---|---|---|---|---|
| Suraclose | 200 | mM | 20-40 | mM |
| Ficoll PM 70 | 10% | w/v | 1-2% | |
| Citrate | 16 | mM | 1.6-3.2 | mM |

| | | |
|---|---|---|
| Phosphate formalin | 2.0% | 0.2-0.4% |
| Tris-Buffered-Saline, pH 6.8 | 1X | 0.1-0.2X |

TABLE 9

| Component | 10X Concentration | | Working Range | |
|---|---|---|---|---|
| Suraclose | 200 | mM | 20-40 | mM |
| Ficoll PM 70 | 10% | w/v | 1-2% | |
| Citrate | 16 | mM | 1.6-3.2 | mM |

| | | |
|---|---|---|
| Formalin calcium | 2.0% | 0.2-0.4% |
| Tris-Buffered-Saline, pH 6.8 | 1X | 0.1-0.2X |

Exemplary formulations of the whole blood buffer composition include the formulations listed in the tables below.

TABLE 10

| Component | 10X Concentration | | Working Range | |
|---|---|---|---|---|
| Suraclose | 200 | mM | 20-40 | mM |
| Ficoll PM 70 | 20% | w/v | 2-4% | |
| Ala-Gln dipeptide | 200 | mM | 20-40 | mM |
| Human serum albumin (HSA) | 2% | w/v | 0.1-0.4% | |
| Adenine | 5.0 | mM | 0.5-1.0 | mM |
| EDTA | 100 | mM | 10 | mM |
| Bax Channel Inhibitor | 0.10 | mM | 0.01-0.02 | mM |

| | | |
|---|---|---|
| Phospho-Buffered-Saline, | 1X | 0.1-0.2X |
| pH 7.2 | | |

TABLE 11

| Component | 10X Concentration | | Working Range | |
|---|---|---|---|---|
| Suraclose | 200 | mM | 20-40 | mM |
| Ficoll PM 70 | 20% | w/v | 2-4% | |
| Ala-Gln dipeptide | 200 | mM | 20-40 | mM |
| BSA (Fraction VIII) | 2% | w/v | 0.2-0.4% | |
| Adenine | 5.0 | mM | 0.5-1.0 | mM |
| Citrate | 16 | mM | 1.6-3.2 | mM |
| Bax Channel Inhibitor | 0.10 | mM | 0.01-0.02 | mM |

| | | |
|---|---|---|
| Tris Buffered Saline, pH 7.2 | 1X | 0.1-0.2X |

TABLE 12

| Ingredient | CAS No | Form | MW |
|---|---|---|---|
| Sucralose | 56038-13-2 | Powder | 397.63 |
| Ficoll PM 70 | 72146-89-5 | Powder | 70,000 |
| Ala-gln | 39537-23-0 | Powder | 217.22 |
| Human Serum Albumin | NA | Crystal | 547.69 |
| Adenine | 73-24-5 | Powder | 135.13 |
| Bax Channel Inhibitor | NA | Lyo. | 540.12 |
| 1X PBS | NA | Solution | |
| K2ETA | 25102-12-9 | Powder | 404 |

TABLE 13
whole blood buffer composition

| BF016 | g/100 mL | |
|---|---|---|
| Sucralose | 8 | |
| Ficoll PM 70 | 20 | |
| Ala-gln | 4 | |
| Human Serum Albumin | 2 | |
| Adenine | 0.2 | |
| Bax Channel Inhibitor | 100 | uL |
| 1X PBS | ~45 | mL |

| | |
|---|---|
| K2ETA | 0 |

In an embodiment, the formulation in Table 13 is admixed with a whole blood sample after blood collection in a standard EDTA tub.

TABLE 14

| BF020 | g/100 mL | |
|---|---|---|
| Sucralose | 8 | |
| Ficoll PM 70 | 20 | |
| Ala-gln | 4 | |
| Human Serum Albumin | 2 | |
| Adenine | 0.2 | |
| Bax Channel Inhibitor | 100 | uL |
| 1X PBS | ~45 | mL |

| | |
|---|---|
| K2ETA | 1.8 |

In an embodiment, the formulation in Table 14 is contained in a tube (prefilled tube) for admixture with a whole blood sample during blood draw/collection.

Typically, Ficoll PM 70 is a macromolecule (long chain sugar) that helps to prevent microclot formation. Typically, Human Serum Albumin (HSA) has an important role in blood; it helps with transport of compounds (hormones, fatty acids, etc) in the blood stream as well as helping to maintain osmotic blood pressure. Typically, Adenine helps to extend the storage life of blood that has been collected — through maintenance of ATP in red blood cells. Typically, Bax Channel Inhibitor helps to prevent mitochondria-mediated apoptosis pathway. Here, in the compositions described, the components have a novel advantageous effect and synergistic effect.

In an embodiment, the whole blood sample is preferably in the range of 0.5 to 50 ml, such as in the range of 1 to 40 ml, such as from 5 to 35 ml or 10 to 30 ml. In an embodiment the sample from a subject is venous whole blood. In an embodiment, the sample was collected in a blood specimen collection tube. In another embodiment, the sample was collected in a blood specimen collection tube containing a solution of, but not limited to, EDTA, sucralose, PBS, or adenosine.

It will be appreciated that concentrates or dilutions of the amounts recited herein may be employed. In general, the relative proportions of the ingredients recited will remain the same. Thus, by way of example, if the teachings call for 30 parts by weight of a Component A, and 10 parts by weight of a Component B, the skilled artisan will recognize that such teachings also constitute a teaching of the use of Component A and Component B in a relative ratio of 3:1. Teachings of concentrations in the examples may be varied within about 25% (or higher) of the stated values and similar results are expected. Moreover, such compositions of the examples may be employed successfully in the present methods to isolate fetal nucleic acids (e.g., cell-free fetal DNA).

As used herein, “ficoll polysaccharide” generically, is defined as a synthetic polymer of sucrose and epichlorohydrin. The ficoll polysaccharide may be any commercially available Ficoll®. In some embodiments, the ficoll polysaccride is Ficoll® PM 70. In some embodiments, Ficoll® PM 70 is present at 16-24% w/v (or 2.2-3.4 mM) in a 10× concentrated stock solution of the reagents. Ficoll® is commonly known as part of Ficoll-Paque, which is used for separation of blood to its components and density gradient centrifugation. Accordingly, ficollpolysaccharide as an unexpected active component of the compositions disclosed herein, which are used in the methods disclosed herein, e.g. for inhibiting phase separation.

Exemplary halogenated sucrose derivatives include sucralose (1,6-dichloro-1,6-dideoxy-(3-β-fructofuranosyl-4-chloro-4-deoxy-α-D-galactopyranoside); trichloronated maltose; 1,6-dichloro-1,6-dideoxy-β-D-fructofuranosyl-4-chloro-4-deoxy-6-O-monododecanoate-α-D-galactopyranoside; 1,6-sichloro-1,6-dideoxy-β-D-fructofuranosyl-4-chloro-4-deoxy-6-O-monotetradecanoate-α-D-galactopyranoside; and combinations thereof. In some embodiments, the halogenated sucrose derivative is sucralose. In some embodiments, sucralose is present at 160-240 mM in a 10× concentrated stock solution of the reagents.

Exemplary human serum albumin includes commercially available human serum albumin, recombinant human serum albumin, human serum albumin fusion proteins, and human serum albumin mutants. Exemplary bovine serum albumin includes BSA Fraction V, commercially available bovine serum albumin, recombinant bovine serum albumin, human bovine albumin fusion proteins, and bovine serum albumin mutants. In some embodiments, human serum albumin is present at 1.6 to 2.4% w/v in a 10× concentrated stock solution of the reagents. In some embodiments, the bovine serum albumin is present at 1.6 to 2.4% w/v in a 10× concentrated stock solution of the reagents.

Exemplary cell apoptosis inhibitors include a Bax channel inhibitor, and endoplasmic reticulum stress inhibitor. The cell apoptosis inhibitor is particularly useful wherein the composition herein is used in relation to CTCs (circulating tumor cells) or other cells that have entered or will likely enter an apoptosis program. Inhibiting cell apoptosis further avoids release and/or lysis of nucleic acids. In some embodiments, the cell apoptosis inhibitor is a Bax channel inhibitor present at 0.08 to 0.12 mM in a 10× concentrated stock solution of the reagents.

Exemplary peptides include a small di- and tri-peptides. In some embodiments, the di-peptide has the amino acid sequence aa-aa, wherein aa is any of the 20 natural or any unnatural amino acids. In some embodiments, the di-peptide is alanine-glutamine. In some embodiments, the di-peptide is glycine-glycine. In some embodiments, the tri-peptide has the amino acid sequence aa-aa-aa, wherein aa is any of the 20 natural or any unnatural amino acids. In some embodiments, the tri-peptide is glycine-glycine-glycine. In some embodiments, the peptide is alanine-glutamine present at 160-240 mM in a 10× concentrated stock solution of the reagents.

Exemplary purines include adenine, guanine, or both. In some embodiments, the purine is adenine. In some embodiments, the purine is guanine. In some embodiments, the purine is adenine present at 4.0-6.0 mM in a 10× concentrated stock solution of the reagents.

Exemplary anticoagulants include ethylenediaminetetraacetic acid (EDTA), hirudin, heparin, and a citrate. In some embodiments, the anticoagulant is sodium citrate. In some embodiments, the anticoagulant is hirudin. In some embodiments, the anticoagulant is EDTA. In some embodiments, the anticoagulant is ethylenediaminetetraacetic acid present at 90 to 110 mM in a 10× concentrated stock solution of the reagents. In some embodiments, the anticoagulant is a citrate present at 12.8 to 19.2 mM in a 10× concentrated stock solution of the reagents.

Exemplary pH buffers include citric acid, tartaric acid, malic acid, sulfosalicylic acid, sulfoisophthalic acid, oxalic acid, borate, CAPS (3-(cyclohexylamino)-1-propanesulfonic acid), CAPSO (3-(cyclohexylamino)-2-hydroxy-1-propanesulfonic acid), EPPS (4-(2-hydroxyethyl)-1-piperazinepropanesulfonic acid), HEPES (4-(2-hydroxyethyl)piperazine-l-ethanesulfonic acid), MES (2-(N-morpholino)ethanesulfonic acid), MOPS (3-(N-morpholino)propanesulfonic acid), MOPSO (3-morpholino-2-hydroxypropanesulfonic acid), PIPES (1,4-piperazinediethanesulfonic acid), TAPS (N[tris(hydroxymethyl)methyl]-3-aminopropanesulfonic acid), TAPSO (2-hydroxy-3-[tris(hydroxymethyl)methylamino]-1-propanesulfonic acid), TES (N-[tris(hydroxymethyl)methyl]-2-aminoethanesulfonic acid), bicine (N,N-bis(2-hydroxyethyl)glycine), tricine (N-[tris(hydroxymethyl)methyl]glycine), tris (tris(hydroxymethyl)aminomethane) and bis-tris (2-[bis(2-hydroxyethyl)amino]-2-(hydroxymethyl)-1,3-propanediol). Selection of a pH buffer is dependent on, for example, the desired pH to be maintained, the nature of the sample, the solvent conditions, other components of the formulation, and other criteria. In some embodiments, the pH buffer is employed at a pH that is within about 0.1, 0.2, 0.3, 0.4, 0.5, 0.6, 0.7, 0.8, 0.9 or 1.0 pH unit of a proton dissociation constant (pKa) that is a characteristic of the buffer. In some embodiments, the composition herein has a pH of about 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9 or 9.0. In some embodiments, a phospho-buffered saline with a pH of 7.2 is present at 0.8 to 1.2× in a 10× concentrated stock solution of the reagents. In some embodiments, a tris-buffered saline with a pH of 7.2 is present at 0.8 to 1.2× in a 10x concentrated stock solution of the reagents.

Articles of Manufacture and Kits

Described herein are articles of manufacture comprising a formulation described herein. Example articles of manufacture include a suitable blood collection tube, container or vessel. In an embodiment, a whole blood buffer composition is contained within a blood collection tube. In an embodiment, the blood collection tube is an evacuated blood collection tube. In an embodiment, the blood collection tube is an evacuated blood tube having less than atmospheric pressure to withdraw a predetermined volume of whole blood. In some embodiments, these articles of manufacture are used in the kits and methods described herein.

Described herein are kits comprising any one of the articles of manufacture described herein and a package insert. In some embodiments, the components of the kit are supplied in a packaging means, such as a compartmentalized plastic enclosure, preferably with a hermetically sealable cover so that the contents of the kit can be sterilized and sealed for storage.

Microfluidic Chip

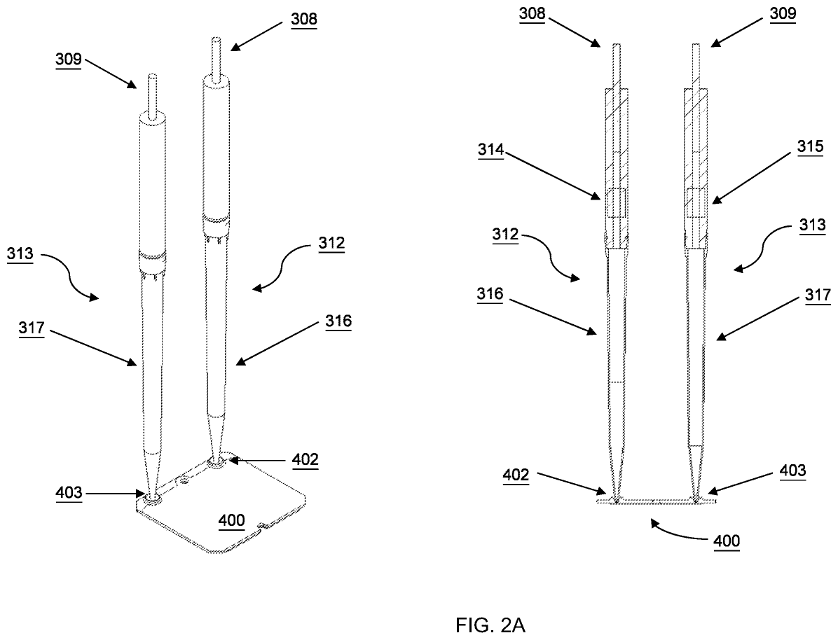

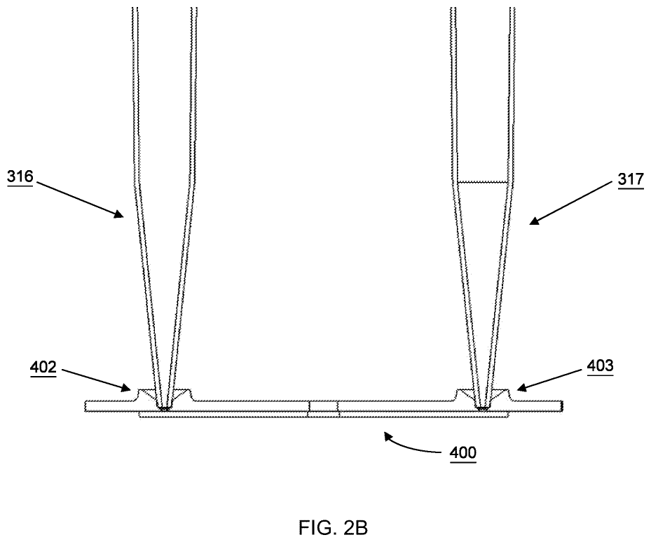

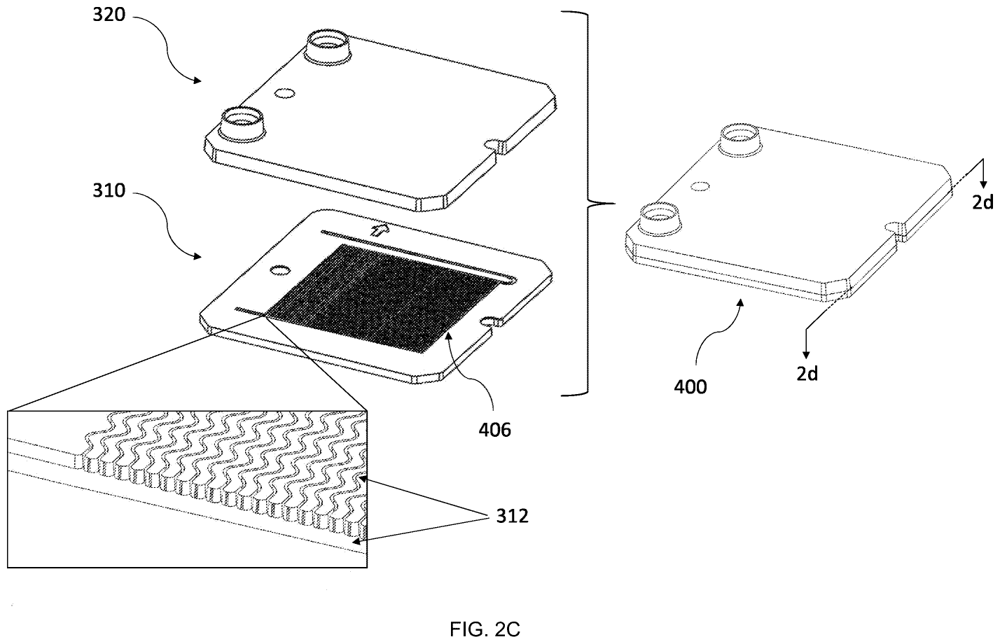

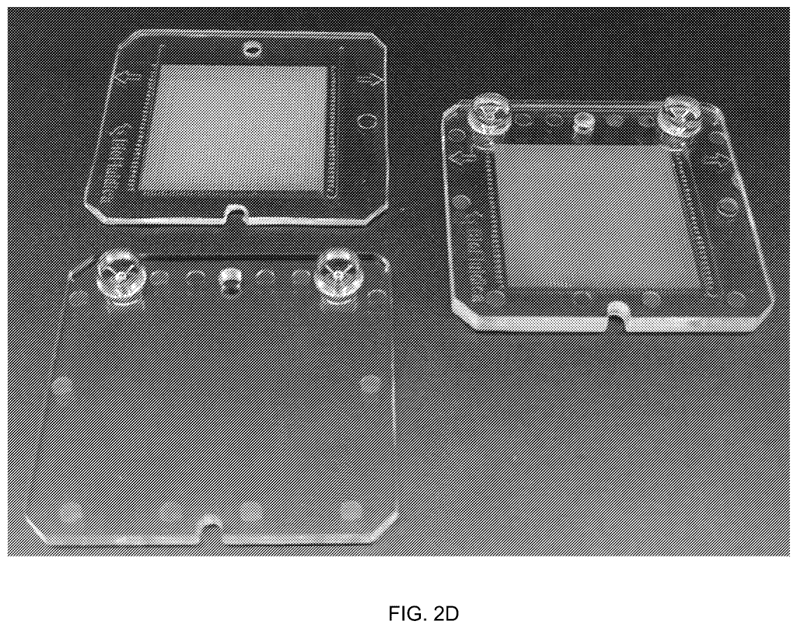

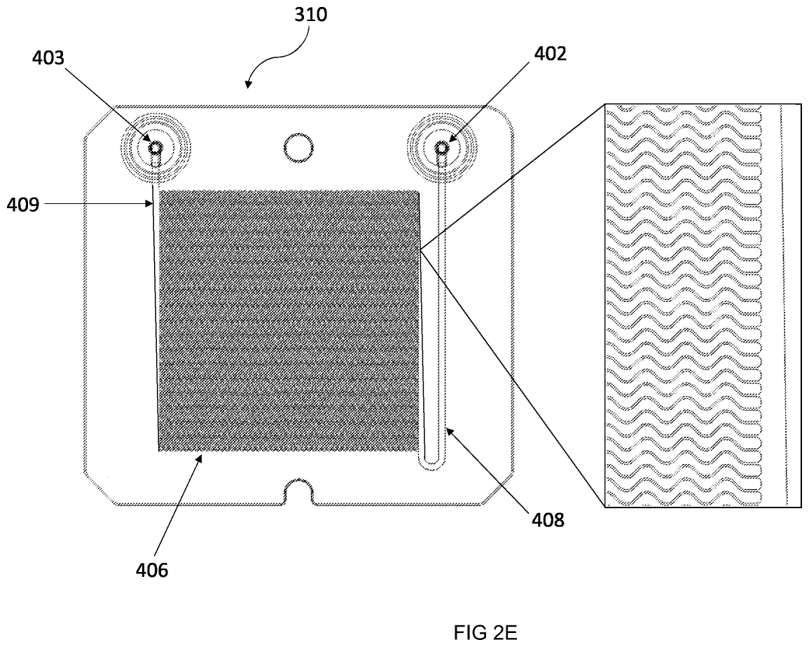

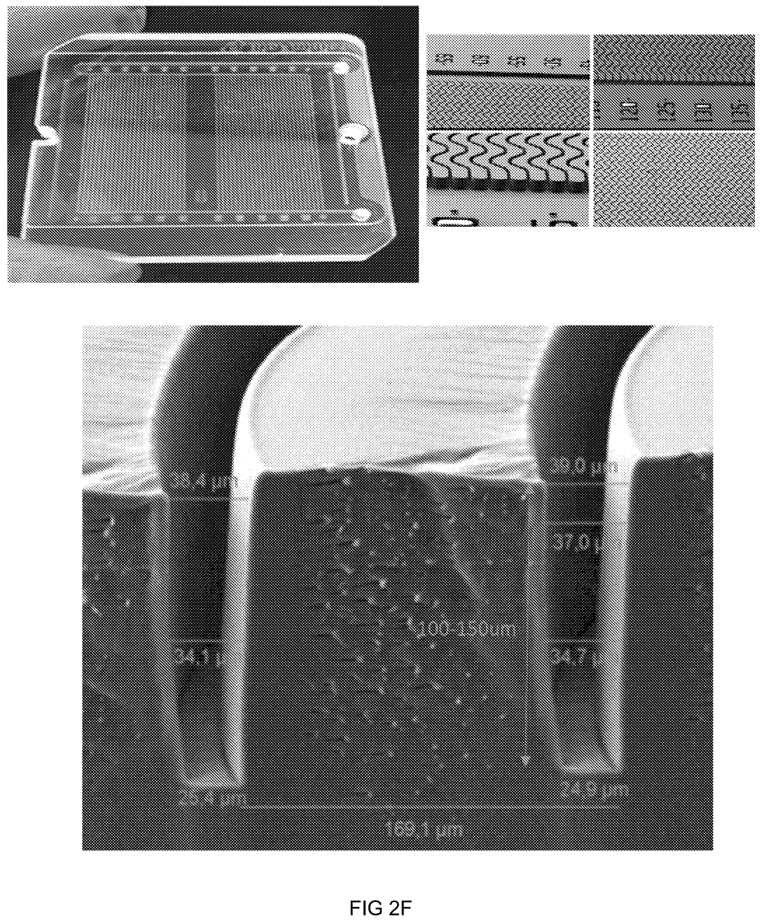

FIG. 2c is an exploded view of the microfluidic chip 400 showing the base plate 310 and cover plate 320 (center), a perspective view of the assembled microfluidic chip 400(right), and a magnified view of channel recesses 312 of the base plate 310 (inset). In some embodiments, the base plate 310 and cover plate 320 is made of a thermoplastic substrate, such as cyclic olefin copolymer (COC), polycarbonate (PC), polymethylmethacrylate, (PMMA), polystyrene (PS), polyvinylchloride (PVC), and polyethyleneterephthalate glycol (PETG). In an embodiment, the thermoplastic substrate is cyclic olefin polymer (COP). The base plate 310 and cover plate 320 can be formed by injection molding or hot embossing techniques known in the art, and then aligned and thermally fusion bonded. As shown, the base plate 310 comprises channel recesses 312 and the cover plate 320 encloses the channel recesses 312 of the base plate 310, thereby forming the channels of the microfluidic chip. In another embodiment, a cover plate comprises channel recesses and a base plate encloses the channel recesses of the cover plate, thereby forming the channels of the microfluidic chip.

FIG. 2d is an image of the base plate (top, left), the cover plate (bottom, left), and the assembled microfluidic chip (right).

FIG. 2e is a cross-section of the microfluidic chip (taken on the plane indicated in FIG. 2c where the base plate and cover plate meet) with a top view of the base plate 310 of the microfluidic chip according to embodiments of the present disclosure (cover plate not shown). The microfluidic chip 400 comprises an inlet port 402, a feeder microchannel 408, multiple parallel sinusoidal channels 406, an exit microchannel 409, and an outlet port 403; wherein the feeder microchannel intersects with the isolation channels, and the exit microchannel intersects with the isolation channels. In one embodiment, the capture bed comprises a plurality of parallel sinusoidal microchannels.

FIG. 2f (top left) is an image of a microfluidic chip 400 with an inlet port 402, outlet port 403, and multiple parallel sinusoidal microchannels 406 with a sinusoidal shape connected to the inlet port and outlet port, and (top right) multiple magnified images of sinusoidal microchannels. FIG. 2f (bottom) is a 100× magnified image of a cross-section of two sinusoidal microchannels of the microfluidic chip 400.

In one embodiment, the microfluidic chip comprises 100 to 150 multiple parallel sinusoidal microchannels. The microchannels have a sinusoidal shape, for example, as shown in FIG. 2f (top right).

In an embodiment, the microfluidic chip comprises microchannels have hydraulic diameters in the range of 10-200 m. In an embodiment, the microfluidic chip comprises microchannels have a channel width or channel height in the range of about 10 to about 200 m.

In one embodiment, the channels have a high aspect ratio. In one embodiment, the channels have a channel width in the range of about 15 μm to about 45 μm, or preferably in the range of about 20 μm to about 25 μm; and a channel height in the range of about 100 μm to about 150 m, or preferably in the range of about 140 μm to about 150 m. In another embodiment, the microchannels have a channel width of about 20 μm and a channel height of about 150 m. The high aspect ratio of the sinusoidal microchannel design maximizes the interaction of the biomarker analyte or cell with the microchannel surface.

In another embodiment, the sinusoidal channels have a tapered width or trapezoidal cross-section shape. In one such embodiment, as shown in FIG. 2f (bottom) the channel width measured at the top of the sinusoidal microchannel is in the range of about 18 μm to about 40 μm and the channel width measured at the bottom of the sinusoidal microchannel is in the range of about 15 μm to about 26 μm. The terms “top” and “bottom” are relative terms to indicate opposite ends of a sinusoidal microchannel cross-section from which the channel width is measured.

The sinusoidal microchannels comprise binding moieties that selectively bind molecules or antigens on membranes of the fetal cells or target analytes. In some embodiments, the binding moieties are antibodies, aptamers, affimers, or haptens immobilized to the interior surface of the sinusoidal microchannels. “Antibody” refers to a polypeptide ligand comprising at least a light chain or heavy chain immunoglobulin variable region which specifically recognizes and binds an epitope of an antigen. Typically, a naturally occurring immunoglobulin has heavy (H) chains and light (L) chains interconnected by disulfide bonds. There are two types of light chain, lambda (λ) and kappa (k). There are five main heavy chain classes (or isotypes) which determine the functional activity of an antibody molecule: IgM, IgD, IgG, IgA and IgE. Examples of antibodies include chimeric antibodies (e.g. humanized murine antibodies), heteroconjugate antibodies (e.g. bi-specific antibodies), fusion proteins, monoclonal antibodies, fully human antibodies.

In some embodiments, the sinusoidal microchannels comprise binding moieties immobilized by or linked with oligonucleotides (containing four uracil-thymidine repeats) to the interior surface of the sinusoidal microchannels. In an embodiment, the oligonucleotide sequence is:

(SEQ ID NO: 1)
5AmMC12-TTTTTTTTCCCTTCCTCCTCACTTCCCTTT-
DideoxyU/T/ideoxy-U/T/ideoxyU/T/ideoxy-
U/TTTTTTTT-3ThioMC3-D

In an embodiment, the oligonucleotide sequence is:

(SEQ ID NO: 2)
5AmMC12-TTTTTTTTCCCTTCCTCCTCACTTCCCTTT-
deoxy-U/T/deoxy-U/T/deoxyU/T/deoxy-U/
TTTTTTTT-3ThioMC3-D

In another embodiment, the oligonucleotide sequence is:

(SEQ ID NO: 3)
5AmMC12-TTTTTTTTCCCTTCCTCCTCACTTCCCTTT-
(deoxy-U/T)ₙ-TTTTTTT-3ThioMC3-D

where n is the number of -deoxy-U/T- repeats and is 1, 2, 3, 4, 5, 6, 7, 8, 9, 10, 11, 12, 13, 14, or 15. Notable features are the dideoxy uracil-thymidine multimer (repeats) which are the target of Uracil DNA Glycosylase. The multimer layout assures that at least one deoxy uracil or dideoxy uracil will be exposed to degradative attack by a USER enzyme or UDG followed by ExoVIII, respectively, which upon the depyrimidination of uracil will cleave the phosphodiester backbone at that location. In some embodiments, the sinusoidal microchannels comprise binding moieties immobilized by or linked with a linear or branched polymer to the interior surface of the sinusoidal microchannels, wherein the linear or branched polymer can be selectively degraded.

In some embodiments, binding moieties that selectively bind to a surface marker of a target analyte (such as fetal cells, circulating tumor cells, exosomes) may combined and immobilized on the microchannel surface at specific ratios. In an embodiment, the ratio of binding moieties that selectively bind to target analyte surface marker A to binding moieties that selectively bind to target analyte surface marker B is 9:1, 8:1, 7:1, 6:1, 5:1, 4:1, 3:1, 2:1, 1:1, 1:2, 1:3, 1:4, 1:5, 1:6, 1:7, 1:8, 1:9. In an embodiment, the ratio of binding moieties that selectively bind to EpCAM to binding moieties that selectively bind to CD105 is 9:1, 8:1, 7:1, 6:1, 5:1, 4:1, 3:1, 2:1, 1:1, 1:2, 1:3, 1:4, 1:5, 1:6, 1:7, 1:8, 1:9. In an embodiment, the ratio of binding moieties that selectively bind to EpCAM to binding moieties that selectively bind to CD105 is 3:1, 2:1, 1:1, 1:2, 1:3. In an embodiment, In an embodiment, the ratio of binding moieties that selectively bind to EpCAM to binding moieties that selectively bind to CD105 is 1:1. In an embodiment, the ratio of binding moieties that selectively bind to EpCAM to binding moieties that selectively bind to CD141 is 9:1, 8:1, 7:1, 6:1, 5:1, 4:1, 3:1, 2:1, 1:1, 1:2, 1:3, 1:4, 1:5, 1:6, 1:7, 1:8, 1:9. In an embodiment, the ratio of binding moieties that selectively bind to EpCAM to binding moieties that selectively bind to CD141 is 3:1, 2:1, 1:1, 1:2, 1:3. In an embodiment, In an embodiment, the ratio of binding moieties that selectively bind to EpCAM to binding moieties that selectively bind to CD141 is 1:1. In an embodiment, sinusoidal microchannels comprise three different kinds of binding moieties, wherein each binding moiety selectively binds to a different target analyte surface marker. In an embodiment, the ratio of binding moiety A: binding moiety B: binding moiety C is 1:1:1.

Liquid Handling System

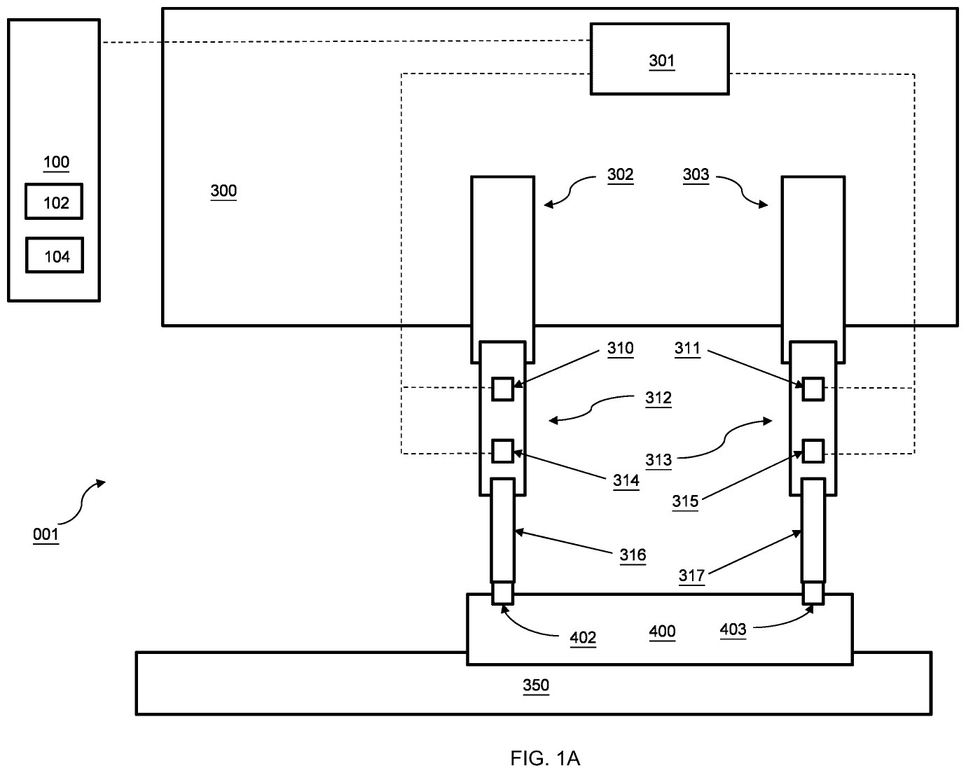

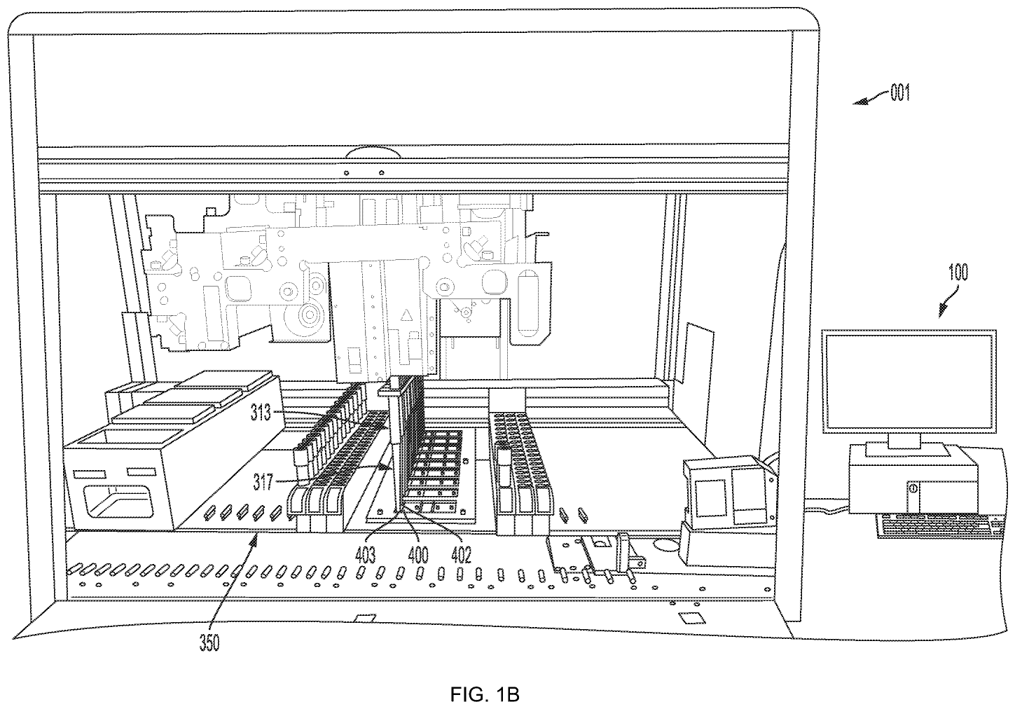

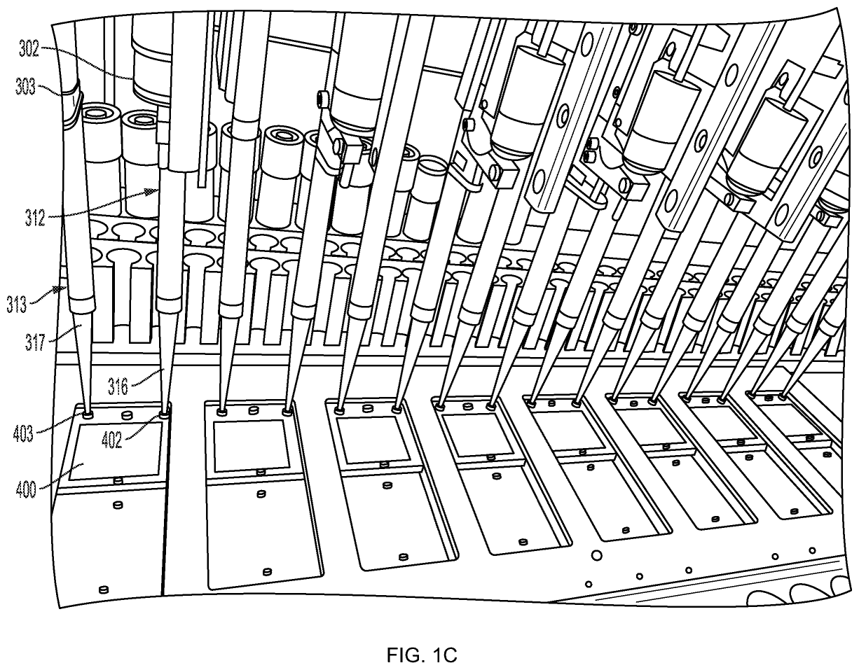

FIG. 1 is a schematic diagram of an example fluid-tight flow system according to embodiments of the present disclosure, and additional components of real-time feedback control according to embodiments of the present disclosure. FIG. 1a illustrates an example fluid-tight flow system including a controller 100, a pipetting instrument 001 comprising two automated pipettes 312 and 313, and a microfluidic chip 400. The microfluidic chip 400 comprises an inlet port 402, an outlet port 403, and multiple parallel sinusoidal channels 406 (shown in FIGS. 2c and 2d). FIG. 1B illustrates an example fluid-tight flow system including a controller 100, e.g. embodied in a computer; a pipetting instrument 001, e.g. an automated liquid handler, comprising multiple automated pipettes, e.g. 312 and 313; multiple microfluidic chips, e.g. 400, each with an inlet port and outlet port, e.g. 402 and 403, respectively; and an instrument deck 350 to support the microfluidic chip 400, pipette tips, samples, reagents, workstations for sample processing. FIG. 1c is a perspective view of multiple pipettes, e.g. 312 and 313, and microfluidic chips, e.g. 400, wherein the pipette tips, e.g. 316 and 317, are coupled to the inlet port, e.g. 402, and outlet port, e.g. 403, of the respective microfluidic chip, e.g. 400, according to embodiments of the present disclosure.

A first automated pipette 312 comprises a pump 308 (shown in FIG. 2a) and a pipette tip 316 that contains a liquid sample (not shown) and is coupled to the inlet port 402. A second automated pipette 313 comprises a pump 309 (shown in FIG. 2a) and a pipette tip 317 that is coupled to the outlet port 403. In one embodiment, the pipette tips 316 and 317 are simultaneously coupled to the inlet port 402 and the outlet port 403, respectively. In one embodiment, the pipette tips 316 and 317 are disposable pipette tips. The two automated pipettes 312 and 313 are configured and operative to control fluid flow of a liquid sample from the pipette tip 316 and through the microfluidic chip 400 via the inlet port 402, multiple parallel sinusoidal channels 406 (shown in FIG. 2c and FIG. 2e), and outlet port 403. The liquid sample may flow through the microfluidic chip into the pipette tip 317 of the second automated pipette 313 or a sample container (not shown).

The pipetting instrument 001 may be an automated liquid handling system such as Biomek™ FX from Beckman-Coulter, Inc. (Brea, Calif.), Freedom EVO™ from Tecan Group, Ltd. (Switzerland), and STAR Line™ from Hamilton Company (Reno, Nev.). In one embodiment, the pipetting instrument 001 comprises an instrument motherboard 301 that is in communication with a controller 100, instrument motors (e.g. pipettor arm drive motors such as X- and Y-drive motors; pipette Z-drive motor; and pipetting drive motors 310, and 311), and instrument sensors (e.g. pressure sensors 314 and 315, tip sensors, capacitive sensors). The instrument motherboard 301 comprises a communication device, a processing device, and a memory device for storing programs that control the functions of various pipetting instrument 001 components. The pipetting instrument 001 may further comprise an instrument deck 350 to support the microfluidic chip 400, pipette tips, samples, reagents, workstations for sample processing.

The controller 100 is in communication with the instrument motherboard 301, instrument motors (e.g. pipettor arm drive motors such as X- and Y-drive motors; pipette Z-drive motor; and pipetting drive motors 310, and 311), and instrument sensors (e.g. pressure sensors 314 and 315, tip sensors, capacitive sensors). In one embodiment, the controller 100 is integrated into the pipetting instrument 001 or with the instrument motherboard 301. The controller 100 generally comprises a communication device, a processing device, and a memory device. The processing device is operatively coupled to the communication device and memory device. The processing device uses the communication device to communicate with the instrument motherboard 301, and as such the communication device generally comprises a modem, server, or other device for communicating with the instrument motherboard 301. The controller 100 may comprises a non-transitory computer readable medium, stored in the memory device, and programmed to command the first pump of the first automated pipette and the second pump of the second automated pipette to control flow of the liquid sample through the microfluidic chip. The controller 100 may be embodied in one or more computers, microprocessors or microcomputers, microcontrollers, programmable logic controllers, field programmable gate arrays, or other suitably configurable or programmable hardware components. The controller 100 may comprise control software, firmware, hardware or other programming instruction sets programmed to receive user inputs, and control instrument motors (e.g. pipettor arm drive motors such as X- and Y-drive motors; pipette Z-drive motor; and pipetting drive motors 310, and 311); as well as provide for real-time feedback control according to embodiments of the present disclosure.

The controller 100 may comprise a non-transitory computer readable medium, stored in the memory device, and programmed to receive data from the first pressure sensor in real-time and data from the second pressure sensor in real-time, and adjust command of at least the first pump of the first automated pipette or the second pump of the second automated pipette to adjust a flow rate within the microfluidic chip using real-time feedback based on said data from the first pressure sensor and second pressure sensor. The controller 100 may comprise control software, firmware, hardware or other programming instruction sets programmed to receive data from instrument sensors (e.g. pressure sensors 314 and 315), receive user inputs, conduct analyses based on pressure data, and adjust control of the pump(s) of the automated pipette(s).

The controller 100 may control parameters of the pipetting instrument 001 such as, timing of movement and X, Y, Z positions of instrument arms 302 and 303, timing and control of pipetting drive motors 310 and 311 such as to control fluid flow rates of a liquid sample through a microfluidic chip. The controller 100 can transmit control signals or other instructions to electrical or electromechanical system components (e.g. such as motors or drives, servos, actuators, racks and pinions, gearing mechanisms, and other interconnected or engaging dynamic parts) via communication technologies to enable data communication (e.g. serial or Ethernet connections, Universal Serial Bus (USB), Institute of Electrical and Electronics Engineers (IEEE) Standard 1394 (i.e., “FireWire”) connections, wireless data communications technologies such as BLUETOOTH™ or other forms based upon infrared (IR) or radio frequency (RF) signals.

FIG. 2a, on the left, is a perspective view of the pipettes 312 and 313 and microfluidic chip 400, wherein the pipette tips 316 and 317 are coupled to the inlet port 402 and outlet port 403 of the microfluidic chip 400, respectively, according to embodiments of the present disclosure; and on the right, is a vertical sectional view of the same according to embodiments of the present disclosure. Accordingly, the pipettes 312 and 313 comprising the pipette tips 316 and 317, respectively, are in fluid communication with the channels of the microfluidic chip. The pipette tips 316 and 317 are coupled to the inlet port 402 and outlet port 403 of the microfluidic chip 400 via a friction fit, thereby creating a hermetic (or air-tight) seal and a leak-tight seal. As used herein, “fluid-tight” means air-tight and leak-tight. In one embodiment, the pumps of the automated pipettes are pistons or plungers 308 and 309 in communication with pipetting drive motors 310 and 311 and pressure sensors 314 and 315. In one embodiment, the pressure sensors 314 and 315 are integrated into the pipettes 312 and 313.

FIG. 2b is a cross sectional view of the pipette tips 316 and 317 coupled to the inlet port 402 and outlet port 403 of the microfluidic chip 400, respectively, according to embodiments of the present disclosure. In one embodiment, the inlet port 402 or outlet port 403 has a tapered shape. In one embodiment, the inlet port 402 and outlet port 403 have a tapered shape and are thus configured to receive and couple to pipette tips are varying sizes.

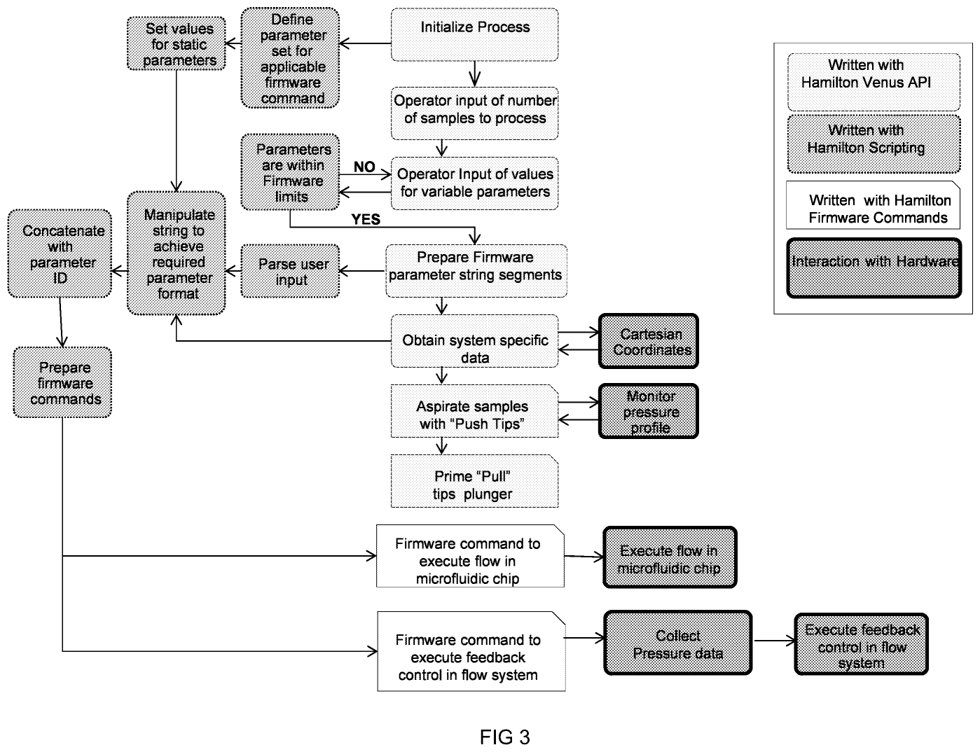

FIG. 3 is the backend software architecture for preparing firmware commands of a Hamilton Microlab STAR line liquid handler according to one embodiment of the present disclosure.

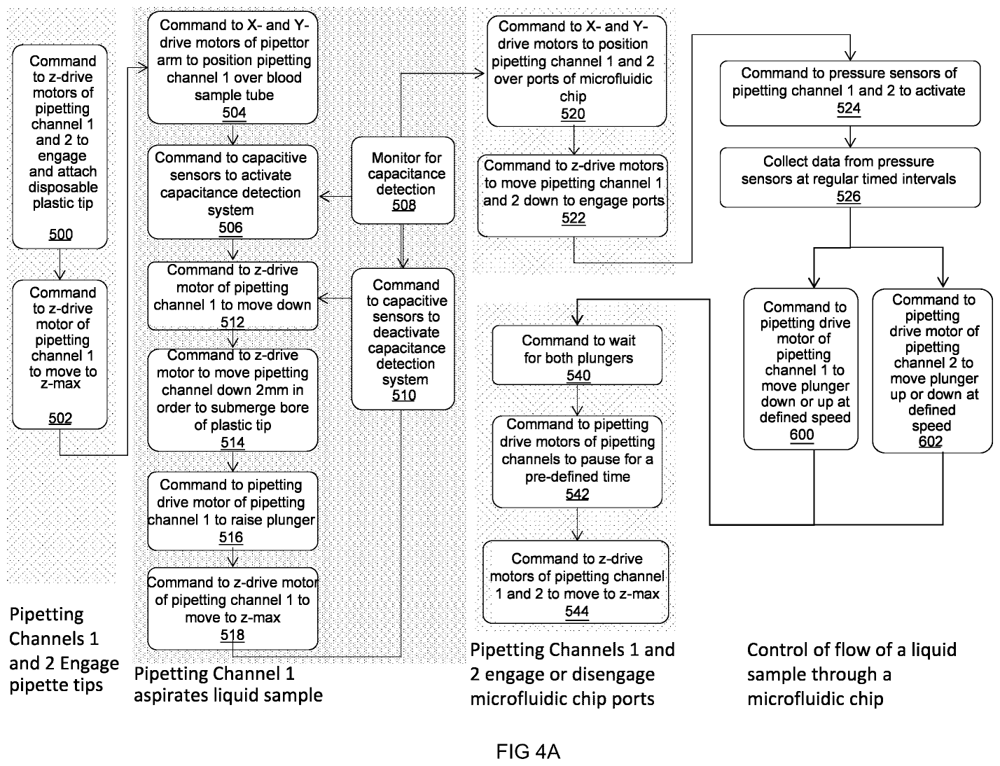

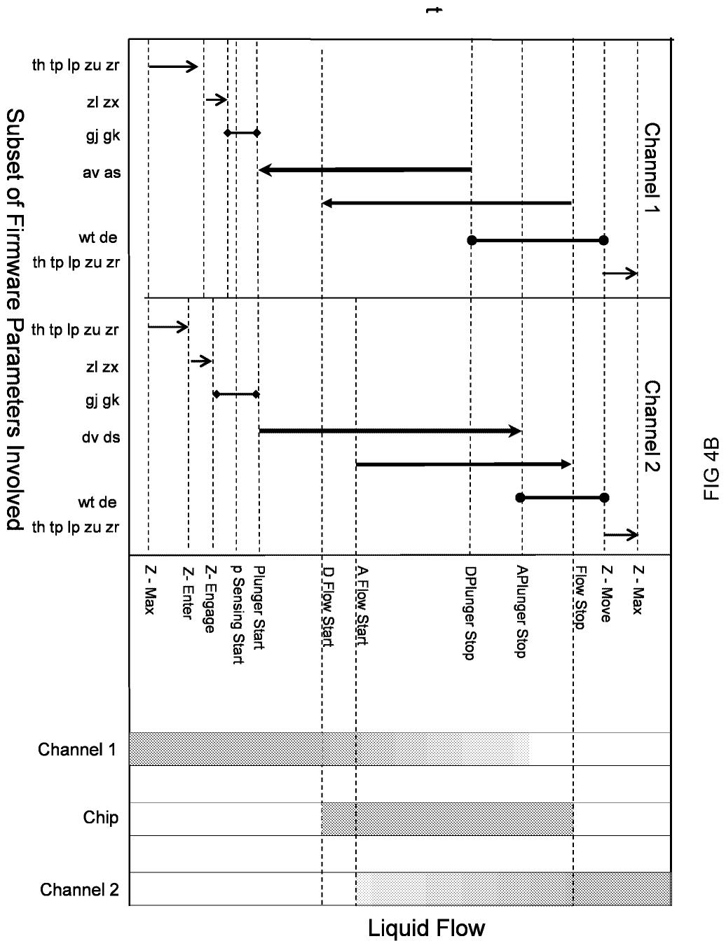

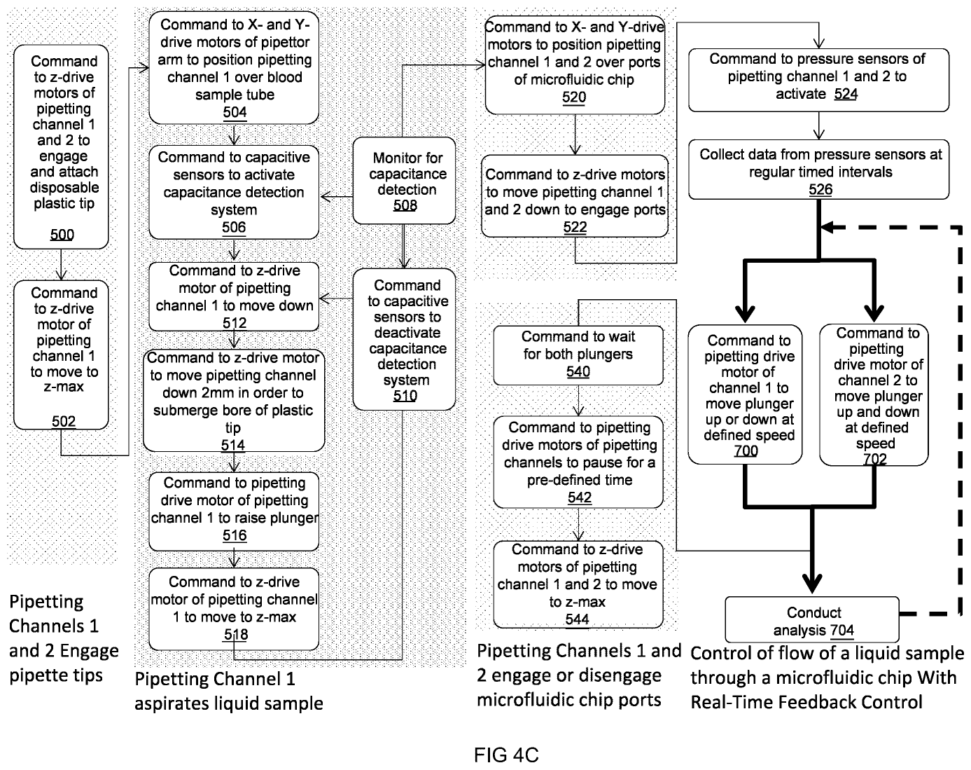

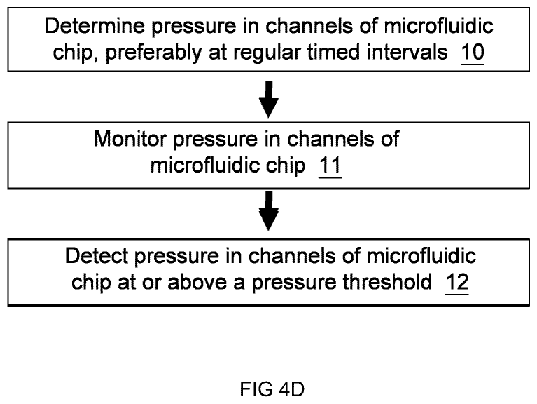

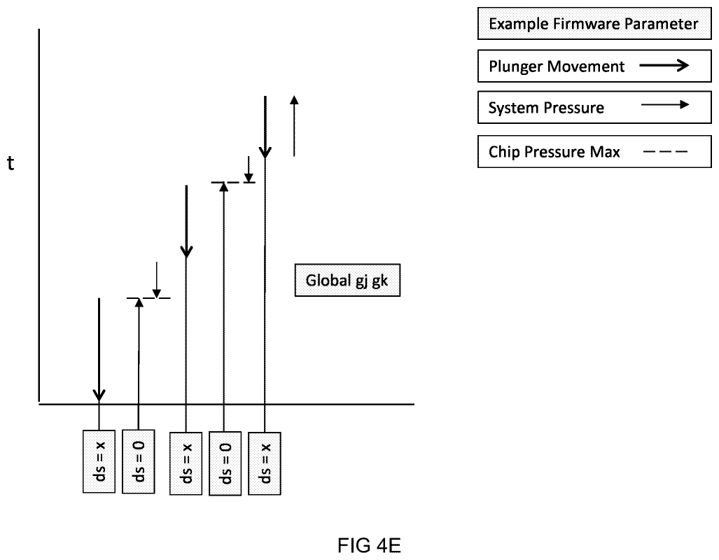

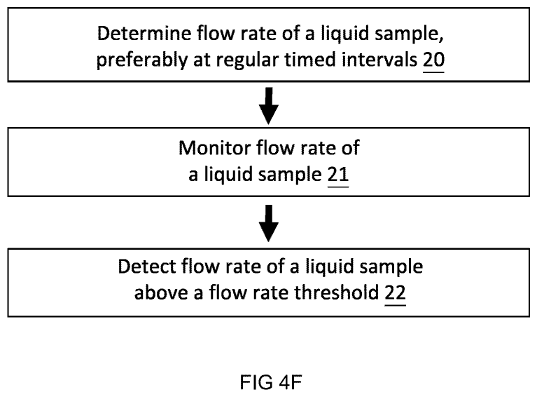

FIG. 4a is a flow chart including exemplary methods according to embodiments of the present invention. The method may be implemented by controller 100 in communication with other components of the presently disclosed system; for example, by sending commands and receiving data via the instrument motherboard 301, which is in communication with instrument motors or instrument sensors. In accordance with some embodiments, a computer readable medium may be encoded with data and instructions for controlling flow of a liquid sample through a microfluidic chip; such as data and instructions to: command the X- and Y-drive motors of the pipetting arm to position pipette 1 and 2, each comprising a pipette tip, over the inlet and outlet ports of a microfluidic chip (step 520), command the z-drive motors to move pipette 1 and 2 down to engage the pipette tips with the inlet and outlet ports of the microfluidic chip, respectively (step 522), command pressure sensors of pipettes 1 and 2 to activate (step 524), collect data from pressure sensors, preferably at regular time intervals (step 526), and command a) pipetting drive motor of pipette 1 to move plunger down or up at a defined speed (step 600) and b) pipetting drive motor of pipette 2 to move plunger up or down at a defined speed (step 602) and coordinate these commands to control flow of a liquid sample through a microfluidic chip and into the pipette tip of pipette 2, and command the z-drive motors of the pipettes to move to z-max (step 544). The command to a pipetting drive motor of a pipette to move plunger down or up at a defined speed includes a defined speed of zero to stop the movement of the plunger. FIG. 4b is an exemplary schematic of coordinating commands and firmware parameters to control z-drive motors and pipetting drive motors of pipettes 1 and 2 to control flow from the pipette tip of pipette 1, through a microfluidic chip, and into the pipette tip of pipette 2, according to embodiments of the present invention.

The fluid-tight flow system reduces the loss of biomaterial by using automated pipettes comprising pipette tips coupled to the inlet and outlet ports of a microfluidic chip, removing extraneous components such as capillary connectors and directly introducing a liquid sample into a microfluidic chip for isolation. Second, the automated pipettes comprising pipette tips coupled to the inlet and outlet ports of a microfluidic chip creates a fluid-tight flow system that enables coordinated use of the pipettes in such a way to control flow of a liquid sample from one pipette tip, through a microfluidic chip, and into the other pipette tip to collect the liquid sample. Typically, pistons or plungers of automated pipettes are configured to aspirate or dispense when the pipette tip is in contact with a liquid sample. The fluid-tight flow system described herein enables use of the pipettes as synchronized pumps to control flow of a liquid sample through a microfluidic chip, including use of a pipette to aspirate or pull a liquid sample that is not in contact with the pipette tip or dispense or push a liquid sample that is no longer in contact with the pipette tip (i.e. when the liquid sample has completely entered the microfluidic chip). Third, the fluid-tight flow system disclosed herein enables control of flow rates at low to extremely low flow rates through microfluidic chips.

FIG. 4c is a flow chart including exemplary methods according to embodiments of the present invention. The method may be implemented by controller 100 in communication with other components of the presently disclosed system; for example, by sending commands and receiving data via the instrument motherboard 301, which is in communication with instrument motors or instrument sensors. In accordance with some embodiments, a computer readable medium may be encoded with data and instructions for controlling flow of a liquid sample through a microfluidic chip; such as data and instructions to: command the X- and Y-drive motors of the pipetting arm to position pipette 1 and 2, each comprising a pipette tip, over the inlet and outlet ports of a microfluidic chip (step 520), command the z-drive motors to move pipette 1 and 2 down to engage the pipette tips with the inlet and outlet ports of the microfluidic chip, respectively (step 522), command pressure sensors of pipettes 1 and 2 to activate (step 524), collect data from pressure sensors, preferably at regular time intervals (step 526), command a) pipetting drive motor of pipette 1 to move plunger down or up at a defined speed (step 700) and b) pipetting drive motor of pipette 2 to move plunger up or down at a defined speed (step 702) and coordinate these commands to control flow of a liquid sample through a microfluidic chip (and ultimately into the pipette tip of pipette 2), conduct analysis on the data from the pressure sensors (step 704) and adjust commands (represented by dotted line) in steps 700 and 702, and command the z-drive motors of the pipettes to move to z-max (step 544). The command to a pipetting drive motor of a pipette to move plunger down or up at a defined speed includes a defined speed of zero to stop the movement of the plunger.

Typically, a pressure sensor monitors pressure in the air space between a liquid sample and a plunger in a pipette. Accordingly, any real-time feedback in current liquid handling pipetting systems with pressure sensors (e.g. Dynamic Device real-time closed loop pipetting systems) is limited to detection of errors related to functions of a pipette tip (e.g. clogging in a pipette tip, flow rate of aspirating into a pipette tip, flow rate of dispensing from a pipette tip, volumetric monitoring of liquid dispensed or aspirated) apart from any fluidic system and thus requiring separate pressure sensors to monitor pressure in a fluidic system. Pressure data and movement of the plunger can be correlated to calculate a standard curve (pressure v. time) representing aspirating a liquid sample into a pipette tip or dispensing a liquid sample from a pipette tip. For example, when the pipette tip is in contact with a sample liquid and as the piston or plunger moves up, air pressure in the tip is lowered and a liquid sample is pushed into a pipette tip by the atmospheric pressure. Deviations from this standard curve can detect errors related to functions of pipette tip, such as a clogged tip during aspiration based on a pressure threshold for clots and incomplete aspiration of a liquid sample into a pipette tip based on a pressure threshold for insufficient liquid in a pipette tip.

The systems and methods including real-time feedback control and disclosed herein have unique advantages in controlling flow in a microfluidic chip. The automated pipettes comprising pressure sensors and pipette tips coupled to the inlet and outlet ports of a microfluidic chip creates a fluid-tight flow system that enables monitoring pressure in a fluidic system and determining flow rate without additional sensor components, and adjusting flow rate with real-time feedback controls. Real-time feedback based on pressure data in the systems disclosed herein comprises detection of clogging in the microfluidic chip, detection of a pressure level at or above a pressure threshold to avoid over-pressure in a microfluidic chip, and detection of flow rate at or above a flow rate threshold for a liquid sample.

FIG. 4d is a flow chart including exemplary methods for conducting analysis on the data from the pressure sensors according to embodiments of the present invention. As shown in FIG. 4d, the step of conducting analysis on the data from the pressure sensors (step 704) can include the following steps, and a computer readable medium may further be encoded with data and instructions to: determine pressure in the channels of a microfluidic chip, preferably at regular timed intervals (step 10), monitor pressure in channels of a microfluidic chip (step 11), and detect pressure in the channels of a microfluidic chip at, above, or below a pressure threshold (step 12). A pressure threshold that correlates to detection of clogging in a microfluidic chip can be determined by 1) comparison between a standard curve (pressure v. time), based on pressure data and movement of the plunger(s), that represents successful flow of a liquid sample through a microfluidic chip and a curve (pressure v. time), based on pressure data and movement of the plunger(s), that represents clogging in a microfluidic chip and 2) selection of a pressure level as a pressure threshold. A pressure threshold that correlates to maximum pressure in a microfluidic chip can be determined by 1) comparison between a standard curve (pressure v. time), based on pressure data and movement of the plunger(s), that represents successful flow of a liquid sample through a microfluidic chip and a curve (pressure v. time), based on pressure data and movement of the plunger(s), that represents reaching maximum pressure in a microfluidic chip and 2) selection of a pressure level as a pressure threshold, e.g. to avoid over-pressure in a microfluidic chip. A computer readable medium may further be encoded with data and instructions to receive user input of a pressure threshold, or to determine any of the foregoing pressure thresholds.

A computer readable medium may further be encoded with data and instructions to repeat adjustments in commands in steps 700 and 702 and analysis (step 704) in order to control flow of a liquid sample through a microfluidic chip with real-time feedback. FIG. 4e is a chart of exemplary real-time feedback control parameters to avoid over-pressure in a microfluidic chip according to embodiments of the present invention. As shown schematically in this chart, steps 10-12 (with respect to a pressure threshold that correlates to maximum pressure in a microfluidic chip), 700, and 702 are repeated over time as fluid flow through a microfluidic chip is adjusted. Pressure thresholds to avoid over-pressure in a microfluidic chip may be defined by user, or a computer readable medium may further be encoded with data and instructions to determine a pressure threshold to avoid over-pressure in a microfluidic chip.

FIG. 4f is a flow chart including exemplary methods for conducting analysis on the data from the pressure sensors according to embodiments of the present invention. As shown in FIG. 4f, the step of conducting analysis on the data from the pressure sensors (step 704) can include the following steps, and a computer readable medium may further be encoded with data and instructions to: determine flow rate of a liquid sample in the channels of a microfluidic chip, preferably at regular timed intervals (step 20), monitor the flow rate of a liquid sample in channels of a microfluidic chip (step 21), and detect a flow rate in the channels of a microfluidic chip at, above, or below a flow rate threshold (step 22). A flow rate threshold that correlates to optimized flow to isolate a given biomarker can be determined by 1) comparison between a standard curve (flow rate v. time), based on pressure data and movement of the plunger(s), that represents successful flow of a liquid sample through a microfluidic chip and a curve (flow rate v. time), based on pressure data and movement of the plunger(s), that represents an optimized flow rate for a class of liquid samples through a microfluidic chip and 2) selection of a flow rate as a flow rate threshold. A computer readable medium may further be encoded with data and instructions to receive user input of a flow rate threshold, or to determine a flow rate threshold. A computer readable medium may further be encoded with data and instructions to repeat adjustments in commands in steps 700 and 702 and analysis (step 704) in order to control flow of a liquid sample through a microfluidic chip with real-time feedback.

As shown in FIG. 1a, the controller 100 comprises a decision engine 102 and a flow control rules server 104. As described herein, a computer readable medium may be encoded with data and instructions to command a) pipetting drive motor of pipette 1 to move plunger down or up at a defined speed (step 700) and b) pipetting drive motor of pipette 2 to move plunger up or down at a defined speed (step 702) and coordinate these commands to control flow of a liquid sample through a microfluidic chip (and ultimately into the pipette tip of pipette 2). The flow control rules server 104 comprises rules for coordinating commands to the pipetting drive motors of the pipettes to control flow of a liquid sample through a microfluidic chip. Exemplary rules are set forth in Table 15:

| | Command to pipetting | Command to pipetting |
|---|---|---|
| | drive motor of Pipetting | drive motor of Pipetting |
| | Channel 1 comprising | Channel 2 comprising |
| Flow Control | pipette tip coupled | pipette tip coupled |
| options | to inlet port | to outlet port |
| Start flow | Move plunger down | Move plunger up |
| Start flow | Move plunger down | No movement of plunger |
| Start flow | No movement of plunger | Move plunger up |
| Decrease flow rate | Move plunger down at decreased | No change in movement |
| | speed | |
| Decrease flow rate | No change in movement | Move plunger up at decrease |
| | | speed |
| Decrease flow rate | No change in movement | Move plunger down at |
| | | decreased speed |
| Decrease flow rate | Move plunger up at decrease speed | No change in movement |
| Decrease flow rate | Move plunger down at decreased | Move plunger up at |
| | speed | decreased speed |
| Increase flow rate | Move plunger down at increased | No change in movement |
| | speed | |
| Increase flow rate | No change in movement | Move plunger up at |
| | | increased speed |
| Increase flow rate | No change in movement | Move plunger down at |
| | | increased speed |
| Increase flow rate | Move plunger up at increased speed | No change in movement |
| Increase flow rate | Move plunger down at increased | Move plunger up at |
| | speed | increased speed |
| Stop flow | Stop movement of plunger | Stop movement of plunger |
| Stop flow | Move plunger down | Move plunger down |
| Stop flow | Move plunger up | Move plunger up |
| Reverse flow | Move plunger in opposite direction | Move plunger in opposite |
| | relative to previous movement | direction relative to previous |
| | | movement |

The flow control rules server 104 may comprise rules for determining a pressure threshold. The flow control rules server 104 may comprise rules for determining a flow rate threshold. The decision engine 102 is configured to determine which rules of the flow control rules server to apply to coordinate commands to the pipetting drive motors of the pipettes to control flow of a liquid sample through a microfluidic chip. In one embodiment, the decision engine 102 is configured to determine which rules of the flow control rules server to apply in response to detection of pressure at, above, or below a pressure threshold. In one embodiment, the decision engine 102 is configured to determine which rules of the flow control rules server to apply in response to detection of a flow rate at, above, or below a flow rate threshold.

The various techniques described herein may be implemented with hardware or software or, where appropriate, with a combination of both. For example, the controller device 100 shown in FIG. 1a may include suitable hardware, software, or combinations thereof configured to implement the various techniques described herein. The methods and system of the disclosed embodiments, or certain aspects or portions thereof, may take the form of program code (i.e., instructions) embodied in tangible media, such as floppy diskettes, CD-ROMs, hard drives, or any other machine-readable storage medium, wherein, when the program code is loaded into and executed by a machine, such as a computer, the machine becomes an apparatus for practicing the presently disclosed subject matter. In the case of program code execution on programmable computers, the computer will generally include a processor, a storage medium readable by the processor (including volatile and non-volatile memory and/or storage elements), at least one input device and at least one output device. One or more programs are preferably implemented in a high level procedural or object oriented programming language to communicate with a computer system. However, the program(s) can be implemented in assembly or machine language, if desired. In any case, the language may be a compiled or interpreted language, and combined with hardware implementations.

The described methods and components of the system may also be embodied in the form of program code that is transmitted over some transmission medium, such as over electrical wiring or cabling, through fiber optics, or via any other form of transmission, wherein, when the program code is received and loaded into and executed by a machine, such as an EPROM, a gate array, a programmable logic device (PLD), a client computer, a video recorder or the like, the machine becomes an apparatus for practicing the presently disclosed subject matter. When implemented on a general-purpose processor, the program code combines with the processor to provide a unique apparatus that operates to perform the processing of the presently disclosed subject matter.

Method of Inhibiting Microclots

Described herein is a method of inhibiting microclotting of a whole blood sample in a microfluidic device comprising admixing a whole blood sample ex vivo with a whole blood buffer composition comprising a ficoll polysaccharide and a halogenated sucrose derivative prior to introduction of the whole blood sample into a microfluidic device comprising microchannels, and hydrodynamically processing the admixed whole blood sample through the microfluidic chip, wherein microclotting in the microfluidic device is inhibited. In an embodiment, the whole blood buffer composition further comprises at least one of a human serum albumin or a bovine serum albumin. In another embodiment, the whole blood buffer composition further comprises a cell apoptosis inhibitor.

In an embodiment, the microchannels have a channel width or channel height in the range of about 10 to about 200 μm. In an embodiment, the microfluidic chip comprises: an inlet port, an outlet port, and microchannels in fluid communication with the inlet port and outlet port.

In an embodiment, the method further comprises providing (a) a liquid handling system for hydrodynamically processing the admixed whole blood sample through the microfluidic chip, wherein the liquid handling system comprises i) a pair of automated pipettes comprising: a) a first automated pipette comprising a first pump, and a first pipette tip containing the admixed whole blood sample, wherein the first pipette tip is coupled to the inlet port, and; b) a second automated pipette comprising a second pump, and a second pipette tip coupled to the outlet port simultaneously with the first pipette tip coupled to the inlet port; and ii) a controller comprising a non-transitory computer readable medium in communication with the first pump and the second pump, and programmed to command the first pump of the first automated pipette and the second pump of the second automated pipette to control flow of the sample through the microfluidic chip.

In an embodiment, the method further comprises providing a pump for hydrodynamically processing the admixed whole blood sample through the microfluidic chip. In an embodiment, the pump is a syringe pump, peristaltic pump, pneumatic pump, or another microfluidic pump that drives or facilitates flow of liquid through a microfluidic device.

Release and Recovery, and Downstream Analysis

The methods described herein advantageously enable recovery of isolated cells or DNA from isolated cells in a simple eluate, which can then be easily transferred for further analysis of the isolated cells or DNA from isolated cells. Known methods, such as cell picking (e.g. with a CytePicker or ALS Cell Celector) or laser capture microdissection, are laborious, time-consuming, and can results in loss of cells. In some embodiments, recovery of the fetal cells occurs in less than 3 hours, 2 hours, or 1 hour. In some embodiments, recovery of the fetal cells occurs in less than 2 hours. These time frames are based on a start time beginning within the start of sample processing through a microfluidic chip according to embodiments of methods described herein.

Cells that do not bind to a binding moiety and are not immobilized can be depleted. In some embodiments, the method comprises flowing buffers or solutions through the microchannels to wash away any cells that have not been isolated or immobilized by a binding moiety. In some embodiments, the method further comprises releasing the isolated cells. In some embodiments, the sinusoidal microchannels comprise binding moieties immobilized by or linked with oligonucleotides containing a uracil residue to the interior surface of the sinusoidal microchannels. In an embodiment, the method further comprises hydrodynamically processing an enzyme to remove or degrade the uracil reside, thereby releasing the isolated cells. In an embodiment, the method comprises recovering the released cells in an eluate or buffer.

In some embodiments, the method further comprises identifying the fetal cells in the eluate. Isolated and recovered fetal cells can be identified by immunocytochemistry methods. Isolated fetal cells are released from a microfluidic device in an eluate and deposited into a collection vessel. The recovered fetal cells may be fixed with 2% Paraformaldehyde or Shandon Green Cytofluid. The fixed fetal cells are deposited onto a slide via cytocentrifugation. The fetal cells on the slide can be stained with a combination of any of the following mouse anti human antibody stains: Cytokeratin marker Pan-CK, CK1, CK2, CK3, CK4, CK5, CK6, CK7, CK8, or CD45. For example, positive staining for two markers and not CD45 is indicative of a fetal derived cell. In another example, multinucleated fetal giant cells can be identified by staining with anti-EpCAM antibodies and/or staining the nuclei or DNA with Hoechst stains.

In some embodiments, the method further comprises enumerating the number of isolated and recovered fetal cells by immunocytochemistry analysis. The number of non-target maternal derived cells from the same cell eluate can also be enumerated by immunocytochemistry analysis. For example, the number of fetal cells per mL of bodily fluid can be determined by counting the number of cells that stain positive for CK-7 and negative for CD-45; and the number of non-fetal cells per mL of bodily fluid can be determined by counting the number of cells that stain positive for CD45. The purity of the cell eluate can be determined by dividing the number of fetal cells by the number of non-fetal cells. These metrics can then be used to evaluate the performance characteristics of the microfluidics device and/or binding moieties and to evaluate a patient sample for screening or diagnostic purposes.

In some embodiments, the method further comprises evaluating the performance of a microfluidic chip and binding moieties. Fetal derived control cell lines are spiked into a bodily fluid specimen derived from a non-pregnant subject. The fetal derived control cells are enriched from the bodily fluid specimen using the microfluidics device surfaced with different combinations of antibodies. DNA can be isolated from the enriched fetal derived control cell lines by incubation with a lysis buffer containing Proteinase K, column based purification, and incubation at 95° C. for 15 minutes; or other methods known in the art. The quantity of DNA isolated is derived from analyzing amplification of a housekeeping gene qPCR against a standard curve of known concentration. Some housekeeping genes include GAPDH, RNaseP, and BACTIN. The quantity of DNA can be correlated with the number of cells present. The correlation of DNA quantity to cell number can be calculated with the assumption that one cell contains 6.6 pg total DNA. The number of non-target cells remaining on the microfluidics device and the percent isolation of non-target cells can be determined by calculating the input number of cells in the starting material by qPCR of the same housekeeping genes and calculating the number of cells in the final elution. The percent capture of the target cells can be determined by dividing the number of cells isolated in the Liquid Scan eluate with the number of starting cells. These metrics can then be used to evaluate the performance characteristics of the microfluidics device and/or binding moieties.

In some embodiments, the method further comprises evaluating the fetal cells from the eluate. For example, utilizing fluorescence in situ hybridization (FISH) analysis, recovered fetal cells can be evaluated for the presence of a Y chromosome specific gene indicating the presence of a male fetus. Recovered fetal cells can also be enumerated and analyzed for fetal chromosome aneuploidy with a FISH analysis. Recovered fetal cells are deposited onto a BSA treated slide via cytocentrifugation. Recovered fetal cells are then fixed on the slides with a combination of Methanol and Acetic Acid and the cells are dehydrated in Ethanol. One or more FISH probe(s) is/are added to the cells. FISH probes specific to a region of the genome can be added to detect the presence of that region in the recovered fetal cells. For example, FISH probes for chromosome enumeration can be added to detect recovered fetal cells which are aneuploid. FISH probes for a Y chromosome specific gene can be added to detect the presence of a male fetus The recovered fetal cells are then imaged after staining with VectaShied+DAPI. The slides are imaged with filters specific to the FISH probes and evaluated.

In some embodiments, the method further comprises lysing the isolated cells, eluting the lysed cells and/or nucleic acids of the lysed cells, and evaluating nucleic acids of the lysed cells. The nucleic acids can be DNA or RNA. In an embodiment, the method comprises lysing the isolated cells by hydrodynamically processing a lysis buffer through said one or more microfluidic chips. Exemplary lysis buffers include a buffer comprising Proteinase K and Sodium Azide, or an RNA specific cell lysis buffer. In an embodiment, the method comprises recovering the nucleic acids from lysed cells in an eluate or buffer. The eluate may be dispensed into a vial or other container. The eluate comprising the nucleic acids can be incubated at a temperature of 25° C.-35° C.-45° C., 45° C.-55° C., 55° C.-65° C., 65° C.-75° C., 75° C. - 85° C., 85° C.-95° C., or 95° C.-105° C. The eluate comprising RNA can be purified through a column.

The eluate comprising the nucleic acids can be evaluated by downstream molecular analysis, including: TaqMan qPCR analysis, amplification of a Y-chromosome specific gene to detect the presence of a male fetus, amplification for STR analysis (e.g. using a commercial forensic analysis kit or an AmpFLSTR kit), amplification of specific regions of the fetal genome with tailored oligonucleotides, amplification of regions of the fetal genome for di-deoxy sequencing, and Next Generation Sequencing. The eluate comprising RNA can be used for downstream gene expression analysis. RNA can be converted to cDNA and analyzed by Next Generation RNA Seq. Nucleic acid testing can be used to identify various fetal characteristics including but not limited to sex of the fetus, preeclampsia in the mother, rhesus status of the fetus and the presence of any chromosomal abnormalities including but not limited to any chromosomal inversions, translocations, aneuploidies, other mutations, or any combination thereof. Other downstream molecular analysis includes polymerase chain reaction (PCR), reverse transcription polymerase chain reaction (RT-PCR), quantitative real time polymerase chain reaction (Q-PCR), gel electrophoresis, capillary electrophoresis, mass spectrometry, fluorescence detection, ultraviolet spectrometry, DNA hybridization, allele specific polymerase chain reaction, polymerase cycling assembly (PCA), asymmetric polymerase chain reaction, linear after the exponential polymerase chain reaction (LATE-PCR), helicase-dependent amplification (HDA), hot-start polymerase chain reaction, intersequence-specific polymerase chain reaction (ISSR), inverse polymerase chain reaction, ligation mediated polymerase chain reaction, methylation specific polymerase chain reaction (MSP), multiplex polymerase chain reaction, nested polymerase chain reaction, solid phase polymerase chain reaction, or any combination thereof.

In an embodiment, the method further comprises providing one or more microfluidic chips for detection or analysis of fetal cells or fetal nucleic acids, wherein each microfluidic chip comprises an inlet port, an outlet port, and microchannels in fluid communication with the inlet port and outlet port; and providing a pair of automated pipettes corresponding to each microfluidic chip, comprising a first automated pipette comprising a first pump, and a first pipette tip containing the eluate or a solution comprising the isolated fetal cells or fetal DNA and coupled to the inlet port; a second automated pipette comprising a second pump, and a second pipette tip coupled to the outlet port; wherein the controller is further programmed to command the first pump of the first automated pipette and the second pump of the second automated pipette to control flow of the eluate or the solution through the microfluidic chip. In an embodiment, the method further comprises hydrodynamically processing the eluate or the solution through said one or more microfluidic chips.

### EXAMPLES:

### Example 1

### Cell Capture of Target Cells Using Fetal Derived Control Cell Lines, Analyzed by a PCR

Materials:

BeWo (ATCC®CCL-98™) male choriocarcinoma placental tissue cell line control cells were retrieved from tissue culture, washed, pelleted and resuspended in 1 mL HBSS. After the addition of 3 μL CellTrace CFSE, the cells were incubated in the dark at room temperature for 20 minutes. The cells were then washed in 25 mL HBSS, pelleted by centrifugation at 150×g for 5 minutes and then resuspended in 1 mL media.

10 μL of the resuspended cells were enumerated on a Countess II FL instrument (Invitrogen, Carlsbad CA) to provide a dilution factor for obtaining 100 cells per mL to spike into whole blood.

22 mL Peripheral blood from a healthy female donor was drawn by venipuncture into a Vacuette® tube containing EDTA. The enumerated BeWo cells were added to the blood sample to a final concentration of 100 cells per mL.

Microfluidic chips made of cyclic olefin polymer (COP) and 42.0 mm×39.1 mm in size were used. (See FIG. 2d) The microfluidic were manufactured from injection molded COP plates that were UV activated and thermally bonded. Each microfluidic chip had an inlet port, an inlet microchannel, 150 sinusoidal channels in parallel, an outlet microchannel, and an outlet port in fluid communication. The chip structures form the microfluidic network and are connected such that a sample would flow through the inlet port, the inlet microchannel, the parallel sinusoidal channels, the outlet microchannel, and the outlet port. The inlet channels, sinusoidal channels, and outlet channels were 25 uM in width and 150 uM in height.

The thermal bonded and UV activated microfluidic chips were prepared with a solution of 20 mg EDC in 1 mL MES combined with 100 μM Oligo solution. The surface of the device was blocked with a 1:1 solution of 20% HSA in 1×PBS followed by treatment with a TCEP in HEPES buffer. The prepared chips were surfaced with one of seven different antibody combinations in triplicate for a total of twenty-one devices. The antibody combinations were 1) EpCAM at 1 mg/mL; 2) CD105 at 1 mg/mL; 3) CD141 at 1 mg/mL; 4) EpCAM+CD105 at 0.5 mg/mL respectively; 5) EpCAM+CD141 at 0.5 mg/mL respectively; CD105+CD141 at 0.5 mg/mL respectively; 6) CD105+CD141 at 0.5 mg/mL respectively; 7) EpCAM+CD105+CD141 at 0.33 mg/mL respectively. EpCAM was sourced from Abcam, and CD105 and CD141 were sourced from Biolegend. The antibodies were immobilized to the interior surface of the sinusoidal microchannels via oligonucleotide linkers containing a uracil residue:

(SEQ ID NO: 4)
5AmMC12-TTTTTTTTCCCTTCCTCCTCACTTCCCTTT-
deoxy-U/T/deoxy-U/T/deoxyU/T/deoxy-U/
TTTTTTTT-3ThioMC3-D
USER enzyme was obtained from (New England
Biolabs).

An isoviscosity buffer composition with the following formulation was prepared:

| Reagent | Volume |
|---|---|
| 20% HSA in 1X PBS | 20 mL |
| 1X PBS | 20 mL |
| BF016 | 4 mL |

BF016 recipe

| Ingredient | Amount/100 mL BF016 | | Final Conc | |
|---|---|---|---|---|
| Sucralose | 8 | g | 201 | mM |
| Ficoll PM 70 | 30 | mL | 2.8 | mM |
| Ala-gln | 10 | mL | 180 | mM |
| Human Serum Albumin | 10 | mL | 10% | (w/v) |
| Adenosine | 10 | mL | 150 | mM |
| Bax Channel Inhibitor | 0.1 | mL | 10 | uM |

| | | | |
|---|---|---|---|
| PBS | ~30 | mL | 1x |

The isoviscosity buffer composition is a buffer to transition between two solutions of extremely differing viscosities, such as blood and PBS. Degassing can occur at interfaces between two miscible liquids of vastly differing viscosity and can be ameliorated by employing a buffer with a viscosity and density in the intermediate range between the two extreme fluids. The isoviscosity buffer is composed in such a way that physiological osmolarity and pH is maintained while viscosities similar to blood are also maintained in order to prevent sudden hydrodynamic pressures in the chip that could lead to cell lysis. Other reagents used include: isoviscosity Buffer 1×PBS, PRF DMEM/5% FBS, HBSS/5% FBS, and HBSS/5% FBS.

Methods:

The LiquidScan liquid handling platform was utilized to perform the liquid handling steps. The LiquidScan liquid handling platform has multiple pairs of automated pipettes (as shown in FIG. 1a and FIG. 1B). Two pipettes (with pipette tips) are employed with each microfluidic chip. Generally, with respect to the fluid chip processing steps below, one pipette aspirates a given fluid, one pipette tip is frictionally coupled to the inlet port and the second pipette tip is frictionally coupled to the outlet port, and the LiquidScan controller coordinates that dispensing and aspiration actions of each pipette in order to process the fluid through the chip. Pipette tips are then disengaged from the inlet port and outlet port and discarded. New pipette tips are used for each fluid.

Exemplary LiquidScan liquid handling platform flow parameters are described in the table below.

| | | | | | | | HBSS/5% | | |
|---|---|---|---|---|---|---|---|---|---|
| | | | | | isoviscosity | PRF | FBS- | | HBSS/5% |
| | PBS | isoviscosity | | Reverse | Buffer 1X | DMEM/5% | wash2 | USER | FBS-Elution |
| | Wash | Buffer | Blood* | Blood | PBS ** | FBS wash1 | ** | Elution | wash |
| Aspirate | 5000 | 1500 | 5000 | 5000 | 1500 | 1500 | 1500 | 400 | 3000 |
| Volume | | | | | | | | | |
| (0.1 mL) | | | | | | | | | |
| Aspirate | 200 | 80 | 10 | 10 | 10 | 10 | 10 | 10 | 20 |
| Speed (0.1 | | | | | | | | | |
| mL/sec) | | | | | | | | | |
| Dispense | 5000 | 1500 | 5000 | 5000 | 1500 | 1500 | 1500 | 400 | 3000 |
| Vol (0.1 | | | | | | | | | |
| mL) | | | | | | | | | |
| Dispense | 200 | 80 | 10 | 10 | 10 | 10 | 10 | 10 | 20 |
| Speed (0.1 | | | | | | | | | |
| mL/sec) | | | | | | | | | |
| Aspirate | 2213 | 2213 | 2213 | 2213 | 2213 | 2213 | 2213 | 2209 | 2213 |
| Height (0.1 | | | | | | | | | |
| mm) | | | | | | | | | |
| Dispense | 2213 | 2213 | 2213 | 2213 | 2213 | 2213 | 2213 | 2209 | 2213 |
| Height (0.1 | | | | | | | | | |
| mm) | | | | | | | | | |
| Blowout | 0 | 0 | 0 | 0 | 0 | 0 | 0 | 0 | 0 |
| Volume | | | | | | | | | |
| (0.1 mL) | | | | | | | | | |
| Settling | 0 | 0 | 0 | 0 | 0 | 0 | 0 | 0 | 0 |
| Time after | | | | | | | | | |
| Aspirating | | | | | | | | | |
| (0.1 s) | | | | | | | | | |
| Settling | 0 | 0 | 0 | 0 | 0 | 0 | 0 | 0 | 0 |
| Time after | | | | | | | | | |
| Dispensing | | | | | | | | | |
| (0.1 s) | | | | | | | | | |
| Global | 45 | 75 | 45 | 45 | 10 | 30 | 20 | 1800 | 120 |
| Settling | | | | | | | | | |
| Time (0.1 s) | | | | | | | | | |
| Mix | 0 | 0 | 0 | 0 | 0 | 0 | 0 | 0 | 0 |
| Volume | | | | | | | | | |
| (mL) | | | | | | | | | |
| Mix Cycles | 0 | 0 | 0 | 0 | 0 | 0 | 0 | 0 | 0 |
| Pulse | 10 | 10 | 150 | 150 | 15 | 15 | 15 | 1 | 1 |
| Number | | | | | | | | | |
| Pulse | 0.1 | 0.1 | 0.1 | 0.1 | 0.1 | 0.1 | 0.1 | 0 | 0 |
| Pause (sec) | | | | | | | | | |
| Ramp | 1 | 1 | 1 | 1 | 1 | 1 | 1 | 1 | 1 |
| Pulse | | | | | | | | | |
| Count | | | | | | | | | |
| Flow Cycles | 1 | 1 | 1 | 1 | 1 | 1 | 1 | 1 | 1 |
| Direction | Backwards | Forward | Forward | Backwards | Forward | Forward | Forward | Forward | Forward |
| of Flow | | | | | | | | | |
| Over | 0 | 0 | 0 | 0 | 0 | 0 | 0 | 0 | 0 |
| Aspirate | | | | | | | | | |
| Dual | 0 | 0 | 0 | 0 | 0 | 0 | 0 | 0 | 0 |
| Collection | | | | | | | | | |
| Source | 1 | 9 | 121 | 121 | 17 | 25 | 33 | 152 | 25 |
| Tube | | | | | | | | | |
| Position | | | | | | | | | |
| Destination | 209 | 209 | 177 | 177 | 233 | 241 | 233 | 233 | 241 |
| Tube | | | | | | | | | |
| Position | | | | | | | | | |
| Pause | | | yes | | yes | | yes | yes | |
| BackFlow | 100 | 100 | 100 | 100 | 0 | 10 | 0 | 0 | 0 |
| Pre (0.1 | | | | | | | | | |
| mL) | | | | | | | | | |
| BackFlow | 450 | 500 | 500 | 500 | 20 | 20 | 20 | 20 | 0 |
| Post (0.1 | | | | | | | | | |
| mL) | | | | | | | | | |
| Tip Type | 1000 | 1000 | 1000 | 1000 | 1000 | 1000 | 1000 | 300 | 1000 |

*Gently swirl or finger tap tubes to resuspend cells that may have settled to bottom
** Clean ports with buffer to remove contaminate blood

The left-hand column generally describes the dispensing and aspirating actions of each pipette to process a given fluid through the chip. Aspirate Volume (0.1 mL) and Aspirate Speed (0.1 mL/sec) refer to pipette 1, and aspiration of a given liquid from a container. Thereafter, pipette 1 couples to the inlet port of the microfluidic chip and pipette 2 simultaneously couples to the outlet port of the same microfluidic chip. Dispense Vol (0.1 mL) and Dispense Speed (0.1 mL/sec) refer to pipette 1, and dispensing of the liquid into the microfluidic chip. Aspirate Height (0.1 mm) refers to Z plane height at which the pipette aspirates. Dispense Height (0.1 mm) refers to Z plane height at which the pipette dispenses. Blowout Volume (0.1 mL) refers to a small volume of air aspirated providing an extra dispense to ensure the tip is completely emptied. Settling Time after Aspirating (0.1 s) refers to the amount of time taken to equilibrate to pressure between the two pipette tips after aspirating. Settling Time after Dispensing (0.1 s) refers to the amount of time taken to equilibrate to pressure between the two pipette tips after dispensing. Global Settling Time (0.1 s) refers to the amount of time taken to equilibrate to pressure between the two pipette tips after aspirating and dispensing. Mix Volume (mL) refers to the volume required to mix liquids. Mix Cycles refers to the number of times each pipette aspirates and dispenses the mix volume. No mix cycles were performed. Pulse Number refers to the number of pulses for the volume being dispensed. Pulse Pause (sec) refers to the time between pulses. Ramp Pulse Count refers to the multiplier for the speed at which the pulse occurs. Flow Cycles refers to the number of time a given liquid is hydrodynamically processed through the microfluidic chip in one direction. Direction of Flow refers to the direction of flow of a given liquid. Over Aspirate refers to the volume overage required to aspirate the sample volume. Dual Collection refers to both channels aspirating simultaneously. Source Tube Position refers to the location on the deck of the aspirated specimen. Destination Tube Position refers to the location on the deck of the dispensed specimen. Pause refers to a direction to the software to stop all motion until manually overridden. BackFlow Pre (0.1 mL) refers to dispensing prior to engaging with the chip. BackFlow Post (0.1 mL) refers to dispensing prior to disengaging with the chip. Tip Type refers to the type of pipette tip (e.g. 1000 refers to a 1000 μL, pipette tip and 300 refers to a 300 μL pipette tip.

In an embodiment, a pulsative flow comprising 5-400 pulses per ml of liquid follows this pattern:
a. a pulse that increases flow rate to 1 μl/sec for 100 milliseconds;
b. pause for 100 milliseconds; and
c. repeat until a given liquid is processed.
   In an embodiment, the pulsative flow comprises 100-400 pulses per milliliter of liquid sample and a repetitive flow pattern comprising an increase in flow rate to 1 μl/sec for 100 milliseconds and a pause for 100 milliseconds. In an embodiment, the pulsative flow comprises 300 pulses per milliliter of maternal sample and a repetitive flow pattern comprising an increase in flow rate to 1 μl/sec for 100 milliseconds and a pause for 100 milliseconds. In an embodiment, the pulsative flow comprises 100 to 400 pulses per milliliter of maternal sample and a repetitive flow pattern comprising an increase in flow rate to a flow rate in a range of 1-5 μl/sec for 100-200 milliseconds and a pause for a period of time in a range from 100 milliseconds to 1 second. In an embodiment, the pulsative flow comprises 50-200 pulses per milliliter of maternal sample and a repetitive flow pattern comprising an increase in flow rate to a flow rate in a range of 1-5 μl/sec for 100-200 milliseconds and a pause for a period of time in a range from 100 milliseconds to 1 second.

The overall flow rate of a given liquid through the microfluidic chip can be calculated by measuring the volume of liquid per unit time and can be adjusted using the pulse pause and global settling time to alter flow dynamics through the chip. In an embodiment, the pulsative flow results in extremely low flow rates, as low as 1 μL-12.5 μL/minute

The top row of the table above describes the liquid to be processed through the microfluidic chip. The PBS wash removes microfluidic chip storage fluid. The isoviscosity buffer composition primes the microfluidic chip after the PBS wash and prior to flowing the blood sample through the microfluidic chip. The blood sample comprised a whole blood buffer composition if the blood draw was more than 24 hrs prior to LiquidScan processing. The blood sample tube may be gently swirled or finger tapped to resuspend cells that may have settled to the bottom of the tube. The blood sample was bidirectionally loaded. The inlet and outlet ports of the microfluidic chip are cleaned with a buffer to remove contaminate blood. The isoviscosity Buffer 1× PBS primes the microfluidic chip after the blood sample is hydrodynamically processed and prior to FBS washes. The PRF DMEM/5% FBS wash and HBSS/5% FBS washes remove red blood cells and other cells that did not bind to a binding moiety. The inlet and outlet ports of the microfluidic chip are cleaned with a buffer to remove contaminate blood. The HBSS/5% FBS-Elution wash flushes the isolated target cells from the chip, thereby recovering the isolated target cells in an eluate. The eluate in the pipette tip can be transferred to a receptacle such as a sterile microtube. If a blood sample was processed in part by more than one microfluidic chip, the eluate comprising the recovered isolated target cells from each chip can be pooled.

The above Liquid Scan flow parameters are exemplary. Flow parameters are adjusted depending on the viscosity of the starting sample, the type of biospecimen, the number of rare cells in the starting sample, the number of binding moieties on the cell surface, and/or the desired purity. These flow parameters are adjusted by changing the aspiration and dispensing volume and speed, the global settling time and the number of pulse pauses. For example, adjustments to flow parameters are also made depending on whether the blood sample was admixed with a whole blood buffer composition, or a dual whole blood and fixative buffer composition. Adjustments to flow protocols are made depending on the type of downstream protocol implemented. For example, for downstream qPCR, cells are lysed on chip with a PicoPure buffer comprising Proteinase K and the buffer and lysed cells are eluted off the chip. For downstream ICC, the linker is enzymatically cleaved with the USER™ enzyme, and the cells were eluted off the chip.

In Example 1: 1 mL BeWo spiked peripheral blood was processed over each pre-prepared microfluidic chip on the LiquidScan liquid handling platform. In brief, the device was flushed with 500 μL 1×PBS prior to equilibration with an isoviscosity buffer. Two times 500 μL blood was bi-directionally loaded through the microfluidic chip for a total of 1 mL blood flowed through each device. A post blood load wash of 150 μL the isoviscosity buffer/1× PBS was followed with two FBS washes. 40 μL PicoPure buffer (Thermo Fisher, Carlsbad, CA) containing Proteinase K was added directly to the washed cells immobilized on the chip. Following a 40 minute incubation on the device at room temperature the buffer, containing the lysed cells, was eluted off the chip by flowing an additional 40 μL PicoPure buffer through the chip.

The eluate was incubated at 65 C for three hours followed by a ten minute incubation at to inactivate the Proteinase K. The eluate was lyophilized in a Savant DNA 120 SpeedVac Concentrator (Thermo Fisher, Carlsbad, CA). Each dried down pellet was resuspended in a Quantifiler™ Duo DNA Quantification Kit reagent mix comprising 42 μL Primer Mix, 50 μL Master Mix and 8 μL DNase/Rnase free water. This kit measures amplification of total DNA with the RPPH1 Gene and the male fetal fraction with the SRY Gene. After a thorough vortex, the resuspended pellet was pipetted into four 25 μL aliquots in a MicroAmp Endura Plate Optical 96-Well Fast Clear Reaction Plate (Applied Biosystems, Foster City, CA).

A standard curve of eight DNA concentrations: 50000 pg/μL; 16700 pg/μL; 5560 pg/μL; 1850 pg/μL; 620 pg/μL; 210 pg/μL; 68 pg/μL and 23 pg/μL was added to the PCR plate and amplified alongside the samples.

The reactions were performed on a Quant Studio 3 Real-Time PCR System under the following cycling conditions: 50 C for 2 minutes, 95 C for 10 minutes followed by 40 cycles of for 15 seconds, 60 C for 1 minute with data collection on.

The quantity of DNA is correlated with the number of cells present. Ct values were converted into sample concentration in pg/μL against the standard curve. The concentration was converted to cells/mL using a conversion factor of 6.6 pg DNA/cell.

Results:

For each microfluidic chip surface condition (left hand column), the table below provides 1) the average number of target fetal cells isolated out of the 100 spiked fetal derived control cells per mL of blood sample, 2) the number of fetal cells isolated using binding moieties other than EpCAM alone divided by the number of fetal cells isolated using EpCAM alone. EpCAM alone is the standard baseline for comparison to assess the performance of the all other antibodies and/or combinations thereo.

| Microfluidic Chip | AVERAGE of | Capture target |
|---|---|---|
| sinusoidal channel | Male Fetal Cells/ | vs. Standard |
| Surface Antibody | Input mL | EpCAM |
| EpCAM | 95 | 1.00 |
| CD105 | 99 | 1.04 |
| CD141 | 31 | 0.33 |
| EpCAM + CD105 | 114 | 1.20 |
| EpCAM + CD141 | 98 | 1.03 |
| CD105 + CD141 | 31 | 0.33 |
| EpCAM + CD105 + CD141 | 36 | 0.38 |

### Example 2

### Cell Capture of Trophoblasts from Patient Samples, analyzed by ICC

Materials:

10 mL Peripheral blood from a female pregnant with a male fetus was drawn by venipuncture into a Vacuette® tube containing EDTA. The blood was diluted in PBS to a final volume of 24 mL.

Samples not processed for cell capture the same day of the blood draw were admixed with a whole blood buffer composition. The whole blood buffer composition was made with the following materials per 100 ml: 8 grams sucralose (from Sigma-Aldrich, PN: 69293, CAS No. 56038-13-2, powder form), 20 grams Ficoll PM 70 (from Sigma-Aldrich, PN: F2878, CAS No. 72146-89-5, powder form), 4 grams Ala-gln dipeptide (from Sigma-Aldrich, PN: A8185, CAS No. 39537-23-0, powder form), 2 grams human serum albumin (from SeraCare, PN: 1850-0026, crystal form), 200 mg adenine (from Sigma-Aldrich, PN: A2786, CAS No. 73-24-5, powder form), 100 μL Bax channel inhibitor (from TOCRIS, PN: 2160, lyophilized form), and about 90 mls of 1× PBS (from Gibco, PN: 10010-023, solution form). A 50 ml batch was prepared. 1 gram of human serum albumin was mixed with 10 grams of Ficoll 70 PM in their powder forms. 3 mls of 1×PBS, Ca²⁺, Mg²⁺ free was added to a 100 ml beaker, then stirred on a magnetic stir plate at medium speed with a magnetic stir bar at least one radius of the beaker in length. Each of the following was added and allowed to completely dissolve: 1 gram of sucralose, 1 gram of the Ficoll PM/HSA powder mixture, and 1 gram of Ala-Glu. Each of the following was added and allowed to completely dissolve (three times): 1 gram of sucralose, and 1 gram of the Ficoll 70 PM/HSA powder mixture. 1 gram of Ala-Glu was added and allowed to completely dissolve. of 1×PBS, Ca²⁺, Mg²⁺ was added and allowed to mix for 30 seconds at an increased stirring speed of about a 10% increase. 1 gram of the Ficoll 70 PM/HSA powder mixture was added and allowed to completely dissolve. The mixture was stirred for 30 minutes. The product will have a slight yellow color and a thick white foam on top. The mixture product was transferred to a 50 ml screw-cap conical tube, carefully allowing the foam to transfer. Solution rest for 10 minutes. 1×PBS, Ca²⁺, Mg²⁺ was added q.s. to 50 mls. The mixture was gently mixed by inversion 10 times. 120 ul of BF016 was added to 1.2 mls of whole blood collected within the last 24 hours. The entire sample was used to perform SEDI-RATE® ESR test to determine the Erythrocyte Sedimentation Rate. The ESR should be approximately 0 at 1 hr and <10 at 24 hours. Scores in this range indicate the buffer is behaving properly.

Microfluidic chips made of cyclic olefin polymer (COP) and 42.0 mm×39.1 mm in size were used. (See FIG. 2d) The microfluidic were manufactured from injection molded COP plates that were UV activated and thermally bonded. Each microfluidic chip had an inlet port, an inlet microchannel, 150 sinusoidal channels in parallel, an outlet microchannel, and an outlet port in fluid communication. The chip structures form the microfluidic network and are connected such that a sample from a pregnant subject would flow through the inlet port, the inlet microchannel, the parallel sinusoidal channels, the outlet microchannel, and the outlet port. The inlet channels, sinusoidal channels, and outlet channels were 25 uM in width and 150 uM in height.

The thermal bonded and UV activated microfluidic chips were prepared with a solution of 20 mg EDC in 1 mL MES combined with 100 μM Oligo solution and then treated with a TCEP in HEPES buffer. The prepared chips were surfaced with one of seven different antibody combinations. Eight chips were prepared for each antibody combination. The antibody combinations were 1) EpCAM at 1 mg/mL; 2) CD105 at 1 mg/mL; 3) CD141 at 1 mg/mL; 4) EpCAM+CD105 at 0.5 mg/mL respectively; 5) EpCAM+CD141 at 0.5 mg/mL respectively; CD105+CD141 at 0.5 mg/mL respectively; 6) CD105+CD141 at 0.5 mg/mL respectively; 7) EpCAM+CD105+CD141 at 0.33 mg/mL respectively. (prophetic). The antibodies were immobilized to the interior surface of the sinusoidal microchannels via oligonucleotide linkers containing a uracil residue.

Methods:

The LiquidScan liquid handling platform was utilized to perform the liquid handling steps. The LiquidScan liquid handling platform has multiple pairs of automated pipettes (as shown in FIG. 1B and FIG. 1c). Two pipettes (with pipette tips) are employed with each microfluidic chip. Generally, with respect to the fluid chip processing steps below, one pipette aspirates a given fluid, one pipette tip is frictionally coupled to the inlet port and the second pipette tip is frictionally coupled to the outlet port, and the LiquidScan controller coordinates that dispensing and aspiration actions of each pipette in order to process the fluid through the chip. Pipette tips are then disengaged from the inlet port and outlet port and discarded. New pipette tips are used for each fluid.

LiquidScan liquid handling platform flow parameters as described in Example 1 are exemplary and were generally used. Flow parameters are adjusted depending on the viscosity of the starting sample, the type of biospecimen, the number of rare cells in the starting sample, the number of binding moieties on the cell surface, and/or the desired purity. These flow parameters are adjusted by changing the aspiration and dispensing volume and speed, the global settling time and the number of pulse pauses. For example, adjustments to flow parameters are also made depending on whether the blood sample was admixed with a whole blood buffer, or a dual whole blood and fixative buffer composition. Adjustments to flow protocols are made depending on the type of downstream protocol implemented. For example, for downstream qPCR, cells are lysed on chip with a PicoPure buffer comprising Proteinase K and the buffer and lysed cells are eluted off the chip. For downstream ICC, the linker is enzymatically cleaved with the USER enzyme, and the cells were eluted off the chip.

In Example 2: 8 mL blood from each pregnant female was processed over 8 pre-prepared microfluidic chips. In brief, the device was flushed with 500 μL 1×PBS prior to equilibration with an isoviscosity buffer. Two times 500 μL blood was bi-directionally loaded through the microfluidic chip for a total of 1 mL blood flowed through each device. A post blood load wash of 150 μL isoviscosity buffer/1×PBS was followed with two FBS washes. 40 uL USER™USER™ enzyme (4 U /10 μL) was added to the device followed by a 30 minute incubation at 37 C. The were eluted for ICC with USER enzyme.

The eluate was mixed with Cytospin Fluid solution (Thermo Fisher Scientific, Waltham, MA) and deposited on a slide via a Cytospin® funnel with a Shandon Cytospin 4 instrument at 750 rpm for 4 minutes. The slides were air dried and treated with 95% ethanol for 20 minutes. The slides were then dried and rehydrated in staining buffer. The specimens were stained using automated processes on the Biocare Intellipath™ FLX stainer with antibodies against CK7 and CD45. After staining, a small drop of Prolong Gold® with DAPI (ThermoFisher, Waltham, MA) was applied to the specimen, mounted with a glass coverslip and visualized using a 20× objective on a Keyence® BZ-X710 All-in-One fluorescence microscope The cell were enumerated using the Keyence BZ-Analyzer software. Cells that were positive for both CK7 and CD45 staining we classified as Trophoblasts. The background was defined by enumerating all cells on the slide. Results:

| | | Av. | Av. |
|---|---|---|---|
| | | Tropho- | Background/ |
| Patient ID | Chip Surface | blasts/mL | mL |
| PN01272123301 | EpCAM | 0.50 | 116.25 |
| | CD105 | TBD | TBD |
| | CD141 | TBD | TBD |
| | EpCAM/CD105 | 2.50 | 135.50 |
| | EpCAM/CD141 | 4.83 | 129.17 |
| | CD105/CD141 | TBD | TBD |
| | EpCAM/CD105/CD141 | TBD | TBD |
| PN01273123401 | EpCAM | 0.75 | 228.75 |
| | CD105 | TBD | TBD |
| | CD141 | TBD | TBD |
| | EpCAM/CD105 | 2.50 | 265.67 |
| | EpCAM/CD141 | 3.00 | 267.83 |
| | EpCAM/CD105/CD141 | TBD | TBD |

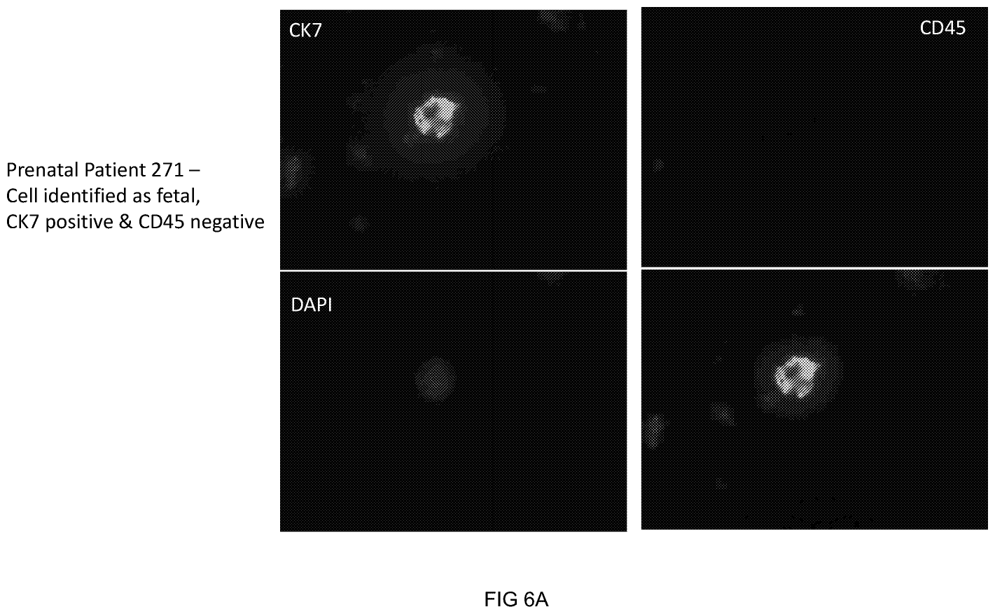

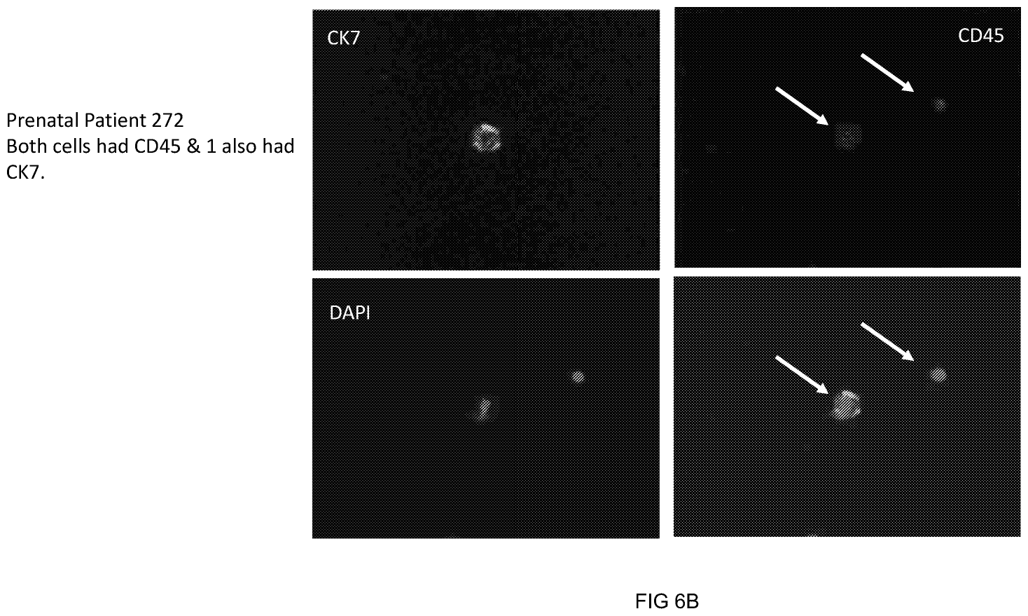

FIG. 6a are images from the fluorescence microscope of a fetal cell from a patient sample that was positive for CK7, negative for CD45.

FIG. 6b are images from the fluorescence microscope of fetal cells from a patient sample—two were positive for CK45, of which one was positive for CD7.

### Example 3

### Cell Capture of Trophoblasts from Patient Samples, Analyzed by a PCR Materials

10 mL Peripheral blood from a female pregnant with a male fetus was drawn by venipuncture into a Vacuette® tube containing EDTA.

Microfluidic chips made of cyclic olefin polymer (COP) and 42 mm×39.1 in size were used. (See FIG. 2d) The microfluidic were manufactured from injection molded COP plates that were UV activated and thermally bonded. Each microfluidic chip had an inlet port, an inlet microchannel, 150 sinusoidal channels in parallel, an outlet microchannel, and an outlet port in fluid communication. The chip structures form the microfluidic network and are connected such that a sample would flow through the inlet port, the inlet microchannel, the parallel sinusoidal channels, the outlet microchannel, and the outlet port. The inlet channels, sinusoidal channels, and outlet channels were 25 uM in width and 150 uM in height.

The thermal bonded and UV activated microfluidic chips were prepared with a solution of 20 mg EDC in 1 mL MES combined with 100 μM Oligo solution and then treated with a TCEP in HEPES buffer. The prepared chips were surfaced with one of seven different antibody combinations. Eight chips were prepared for each antibody combination. The antibody combinations were 1) EpCAM at 1 mg/mL; 2) CD105 at 1 mg/mL; 3) CD141 at 1 mg/mL; 4) EpCAM+CD105 at 0.5 mg/mL respectively; 5) EpCAM+CD141 at 0.5 mg/mL respectively; CD105+CD141 at 0.5 mg/mL respectively; 6) CD105+CD141 at 0.5 mg/mL respectively; 7) EpCAM+CD105+CD141 at 0.33 mg/mL respectively. The antibodies were immobilized to the interior surface of the sinusoidal microchannels via oligonucleotide linkers containing a uracil residue.

Methods:

The LiquidScan liquid handling platform has multiple automated pipettes (as shown in FIG. 1a and FIG. 1B). Two pipettes (with pipette tips) are employed with each microfluidic chip. Generally, with respect to the fluid chip processing steps below, one pipette aspirates a given fluid, one pipette tip is frictionally coupled to the inlet port and simultaneously the second pipette tip is frictionally coupled to the outlet port, and the LiquidScan controller coordinates that dispensing and aspiration actions of each pipette in order to process the fluid through the chip. Pipette tips are then disengaged from the inlet port and outlet port and discarded. New pipette tips are used for each fluid.

LiquidScan liquid handling platform flow parameters as described in Example 1 are exemplary and were generally used. Flow parameters are adjusted depending on the viscosity of the starting sample, the type of biospecimen, the number of rare cells in the starting sample, the number of binding moieties on the cell surface, and/or the desired purity. These flow parameters are adjusted by changing the aspiration and dispensing volume and speed, the global settling time and the number of pulse pauses. For example, adjustments to flow parameters are also made depending on whether the blood sample was admixed with a whole blood buffer composition, or a dual whole blood and fixative buffer composition. Adjustments to flow protocols are made depending on the type of downstream protocol implemented. For example, for downstream qPCR, cells are lysed on chip with a PicoPure buffer comprising Proteinase K and the buffer and lysed cells are eluted off the chip. For downstream ICC, the linker is enzymatically cleaved with the USER™ enzyme, and the cells were eluted off the chip.

In Example 3: 8 mL blood from each pregnant female was processed over 8 pre-prepared microfluidic chips. In brief, the device was flushed with 500 μL 1×PBS prior to equilibration with an isoviscosity buffer. Two times 500 μL blood was bi-directionally loaded through the microfluidic chip for a total of 1 mL blood flowed through each device. A post blood load wash of 150 μL the isoviscosity buffer/1×PBS was followed with two FBS washes. 40 μL PicoPure buffer (Thermo Fisher, Carlsbad, CA) containing Proteinase K was added directly to the washed cells immobilized on the chip. Following a 40 minute incubation on the device at room temperature the buffer, containing the lysed cells, was eluted off the chip by flowing an additional μL PicoPure buffer through the chip.

The eluate was incubated at 65 C for three hours followed by a ten minute incubation at to inactivate the Proteinase K. The eluate was lyophilized in a Savant DNA 120 SpeedVac Concentrator (Thermo Fisher, Carlsbad, CA). Each dried down pellet was resuspended in a Quantifiler™ Duo DNA Quantification Kit reagent mix comprising 42 μL Primer Mix, 50 μL Master Mix and 8 μL DNase/RNase free water. This kit measures amplification of total DNA with the RPPH1 Gene and the male fetal fraction with the SRY Gene. After a thorough vortex, the resuspended pellet was pipetted into four 25 μL aliquots in a MicroAmp Endura Plate Optical 96-Well Fast Clear Reaction Plate (Applied Biosystems, Foster City, CA).

A standard curve of eight DNA concentrations: 50000 pg/μL; 16700 pg/μL; 5560 pg/μL; 1850 pg/μL; 620 pg/μL; 210 pg/μL; 68 pg/μL and 23 pg/μL was added to the PCR plate and amplified alongside the samples.

The reactions were performed on a Quant Studio 3 Real-Time PCR System under the following cycling conditions: 50 C for 2 minutes, 95 C for 10 minutes followed by 40 cycles of for 15 seconds, 60 C for 1 minute with data collection on.

Ct values were converted into sample concentration in pg/μL against the standard curve. The concentration was converted to cells/mL using a conversion factor of 6.6 pg DNA/cell.

Results:

TaqMan quantitative PCR protocol were followed to test for the presence of the Y chromosome in female blood patient samples spiked with male control cells.

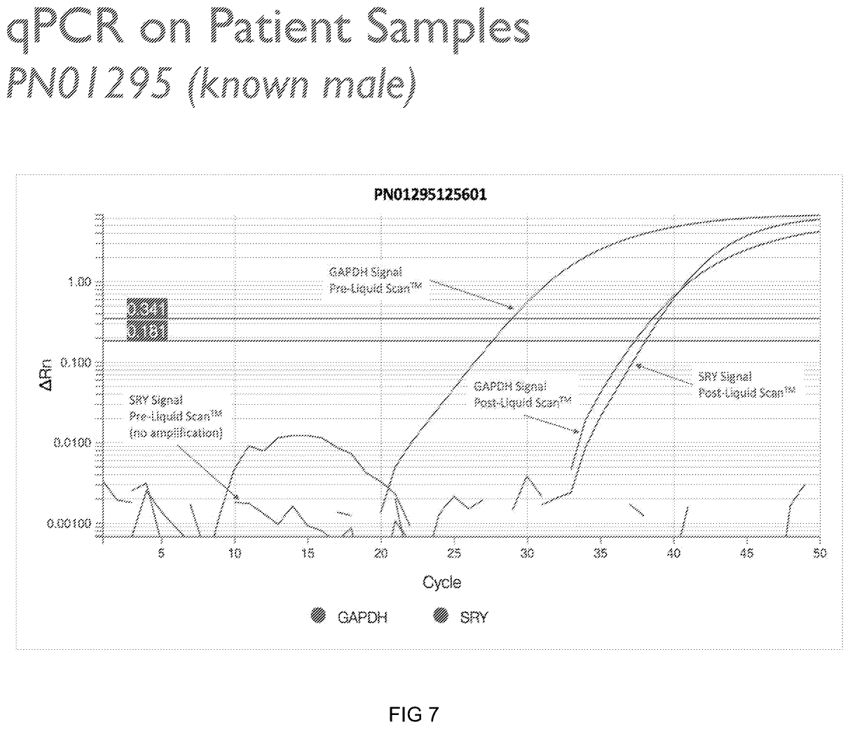

FIG. 7b is a graph showing qPCR data on patient sample PN01295.

### Prophetic Example 4

### Cell Capture of Trophoblasts from Control Cell Lines, Analyzed by ICC

Materials:

BeWo (ATCC®CCL-98 TH) male choriocarcinoma placental tissue cell line control cells were retrieved from tissue culture, washed, pelleted and resuspended in 1 mL HBSS. After the addition of 3μL CellTrace CFSE, the cells were incubated in the dark at room temperature for 20 minutes. The cells were then washed in 25 mL HBSS, pelleted by centrifugation at 150×g for 5 minutes and then resuspended in 1 mL media.

10 μL of the resuspended cells were enumerated on a Countess II FL instrument (Invitrogen, Carlsbad CA) to provide a dilution factor for obtaining 100 cells per mL to spike into whole blood.

22 mL Peripheral blood from a healthy female donor was drawn by venipuncture into a Vacuette® tube containing EDTA. The enumerated BeWo cells were added to the blood sample to a final concentration of 100 cells per mL.

Microfluidic chips made of cyclic olefin polymer (COP) and 42.0 mm x 39.1 mm in size were used. (See FIG. 2d) The microfluidic were manufactured from injection molded COP plates that were UV activated and thermally bonded. Each microfluidic chip had an inlet port, an inlet microchannel, 150 sinusoidal channels in parallel, an outlet microchannel, and an outlet port in fluid communication. The chip structures form the microfluidic network and are connected such that a sample would flow through the inlet port, the inlet microchannel, the parallel sinusoidal channels, the outlet microchannel, and the outlet port. The inlet channels, sinusoidal channels, and outlet channels were 25 in width and 150 in height.

The thermal bonded and UV activated microfluidic chips were prepared with a solution of 20 mg EDC in 1 mL MES combined with 100 μM Oligo solution and then treated with a TCEP in HEPES buffer. The prepared chips were surfaced with one of seven different antibody combinations in triplicate for a total of twenty-one devices. The antibody combinations were 1) EpCAM at 1 mg/mL; 2) CD105 at 1 mg/mL; 3) CD141 at 1 mg/mL; 4) EpCAM+CD105 at 0.5 mg/mL respectively; 5) EpCAM+CD141 at 0.5 mg/mL respectively; CD105+CD141 at 0.5 /mL respectively; 6) CD105+CD141 at 0.5 mg/mL respectively; 7) EpCAM+CD105+CD141 at 0.33 mg/mL respectively. The antibodies were immobilized to the interior surface of the sinusoidal microchannels via oligonucleotide linkers containing a uracil residue.

Methods:

The LiquidScan liquid handling platform has multiple automated pipettes (as shown in FIG. 1a and FIG. 1b). Two pipettes (with pipette tips) are employed with each microfluidic chip. Generally, with respect to the fluid chip processing steps below, one pipette aspirates a given fluid, one pipette tip is frictionally coupled to the inlet port and simultaneously the second pipette tip is frictionally coupled to the outlet port, and the LiquidScan controller coordinates that dispensing and aspiration actions of each pipette in order to process the fluid through the chip. Pipette tips are then disengaged from the inlet port and outlet port and discarded. New pipette tips are used for each fluid.

LiquidScan liquid handling platform flow parameters as described in Example 1 are exemplary and were generally used. Flow parameters are adjusted depending on the viscosity of the starting sample, the type of biospecimen, the number of rare cells in the starting sample, the number of binding moieties on the cell surface, and/or the desired purity. These flow parameters are adjusted by changing the aspiration and dispensing volume and speed, the global settling time and the number of pulse pauses. For example, adjustments to flow parameters are also made depending on whether the blood sample was admixed with a whole blood buffer composition, or a dual whole blood and fixative buffer composition. Adjustments to flow protocols are made depending on the type of downstream protocol implemented. For example, for downstream qPCR, cells are lysed on chip with a PicoPure buffer comprising Proteinase K and the buffer and lysed cells are eluted off the chip. For downstream ICC, the linker is enzymatically cleaved with the USER™ enzyme, and the cells were eluted off the chip.

In Example 4: 1 mL BeWo spiked peripheral blood was processed over each pre-prepared microfluidics chip on the LiquidScan liquid handling platform. In brief, the device was flushed with 500 μL 1×PBS prior to equilibration with a viscosity matched buffer. Two times 500 μL blood was bi-directionally loaded through the microfluidic chip for a total of 1 mL blood flowed through each device. A post blood load wash of 150 μL isoviscosity buffer/1×PBS was followed with two FBS washes. 40uL USER (4 U /10 μL) was added to the device followed by a minute incubation at 37 C. Cells were eluted for ICC with HB SS/5% FBS-Elution wash.

The eluate was mixed with Cytospin Fluid solution (Thermo Fisher Scientific, Waltham, MA) and deposited on a slide via a Cytospin® funnel with a Shandon Cytospin 4 instrument at 750 rpm for 4 minutes. The slides were air dried and treated with 95% ethanol for 20 minutes. The slides were then dried and rehydrated in staining buffer. The specimens were stained using automated processes on the Biocare Intellipath™ FLX stainer with antibodies against CK7 and CD45. After staining, a small drop of Prolong Gold® with DAPI (ThermoFisher, Waltham, MA) was applied to the specimen, mounted with a glass coverslip and visualized using a 20× objective on a Keyence® BZ-X710 All-in-One fluorescence microscope The cell were enumerated using the Keyence BZ-Analyzer software. Cells that were positive for both CK7 and CD45 staining we classified as Trophoblasts. The background was defined by enumerating all cells on the slide.

### Example 5

### Fluorescence In Situ Hybridization: Identification of Multinucleated Fetal Giant Cells and Aneuploidy Analysis in Patient Samples

10 mL Peripheral blood from a pregnant female pregnant was drawn by venipuncture into a Vacuette® tube containing EDTA.

Microfluidic chips made of cyclic olefin polymer (COP) and 42.0 mm×39.1 mm in size were used. (See FIG. 2d) The microfluidic chips were manufactured from injection molded COP plates that were UV activated and thermally bonded. Each microfluidic chip had an inlet port, an inlet microchannel, 150 sinusoidal channels in parallel, an outlet microchannel, and an outlet port in fluid communication. The chip structures form the microfluidic network and are connected such that a sample from a pregnant subject would flow through the inlet port, the inlet microchannel, the parallel sinusoidal channels, the outlet microchannel, and the outlet port. The inlet channels, sinusoidal channels, and outlet channels were 25 uM in width and 150 uM in height.

The thermal bonded and UV activated microfluidic chips were prepared with a solution of 20 mg EDC in 1 mL MES combined with 100 μM Oligo solution and then treated with a TCEP in HEPES buffer. EpCAM antibodies were immobilized to the interior surface of the sinusoidal microchannels via oligonucleotide linkers containing a uracil residue.

Methods:

The LiquidScan liquid handling platform has multiple automated pipettes. Two pipettes (with pipette tips) are employed with each microfluidic chip. Generally, with respect to the fluid chip processing steps below, one pipette aspirates a given fluid, one pipette tip is frictionally coupled to the inlet port and simultaneously the second pipette tip is frictionally coupled to the outlet port, and the LiquidScan controller coordinates that dispensing and aspiration actions of each pipette in order to process the fluid through the chip. Pipette tips are then disengaged from the inlet port and outlet port and discarded. New pipette tips are used for each fluid.

LiquidScan liquid handling platform flow parameters as described in Example 1 are exemplary and were generally used. Flow parameters are adjusted depending on the viscosity of the starting sample, the type of biospecimen, the number of rare cells in the starting sample, the number of binding moieties on the cell surface, and/or the desired purity. These flow parameters are adjusted by changing the aspiration and dispensing volume and speed, the global settling time and the number of pulse pauses. For example, adjustments to flow parameters are also made depending on whether the blood sample was admixed with a whole blood buffer composition, or a dual whole blood and fixative buffer composition. Adjustments to flow protocols are made depending on the type of downstream protocol implemented. For example, for downstream qPCR, cells are lysed on chip with a PicoPure buffer comprising Proteinase K and the buffer and lysed cells are eluted off the chip. For downstream ICC, the linker is enzymatically cleaved with the USER™ enzyme, and the cells were eluted off the chip.

In Example 5: 10 mL blood from each pregnant female was processed over 8 pre-prepared microfluidic chips. In brief, the device was flushed with 500 μL 1×PBS prior to equilibration with a viscosity matched buffer. Two times 500 μL blood was bi-directionally loaded through the microfluidic chip for a total of 1 mL blood flowed through each device. A post blood load wash of 150 μL isoviscosity buffer/1×PBS was followed with two FBS washes. 40 uL USER (4 U /10 μL ) was added to the device followed by a 30 minute incubation at 37 C. The were eluted for FISH in HB SS/5% FBS-Elution wash.

The cell eluate was deposited onto a BSA pretreated slide via a Cytospin® funnel on the Cytospin 4 instrument at 1100 rpm for 7 minutes. The cells were fixed onto the slide with Carnoy's solution and then dehydrated in an ethanol series prior to application of a pre-warmed chromosome enumeration probe (CEP) for chromosome X, Y, and 18 and locus specific identifier (LSI) probes for chromosomes 13 and 21. Following a two minute denaturation at 75 C, a coverslip was applied prior to overnight incubation in a 37 C humidifier.

Following removal of the coverslip, the slides were washed in 0.04× SSC at 72 C and 2× SSC containing 0.05% tween-20 at room temperature. After dehydration in an ethanol series, VectaShield with DAPI was applied to the slide prior to imaging on a Keyence® BZ-X710 All-in-One fluorescence microscope.

Results:

A total of 28 cells, which demonstrated triplicate chromosomal complement, were identified (Figure X). Additionally, 13 cells were identified where the number of chromosomes was above that expected for triploidy which represent multinucleate cells originating from the syncytiotrophoblast layer (Figure X).

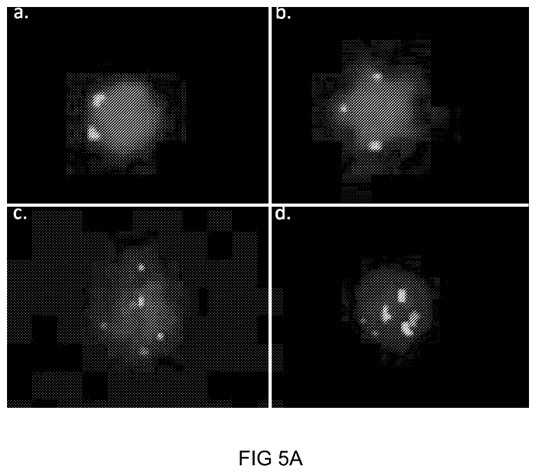

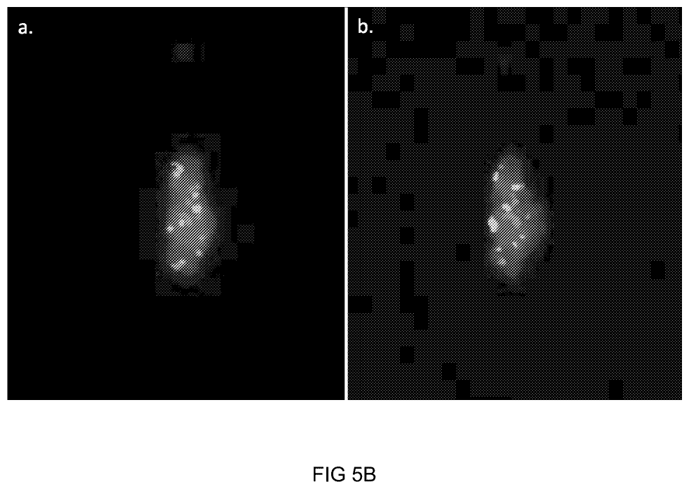

FIG. 5a are images from the fluorescence microscope of: a) a maternal cell showing FISH CEP probe signals on two X chromosomes (only CEP chromosome X aqua probe for DXZ1, Xp11.1-q11.1 were detected; CEP probes for both X and Y chromosomes were used); b) a fetal cell showing FISH CEP probe signals on three X chromosomes (only CEP chromosome X aqua probe for DXZ1, Xp11.1-q11.1 were detected; CEP probes for both X and Y chromosomes were used); c) a fetal cell showing FISH LSI probe signals (3 each) for chromosome 21 and chromosome 13, and d) a fetal cell showing FISH LSI probe signals (3 each) for chromosome 21 and chromosome 18. The maternal peripheral blood mononuclear cell size was about —8 μm in diameter as measured along the longest axis, and each of the fetal trophoblast cells were about 15-30 μm in diameter as measured along the longest axis and had a roughly spherical shape. The trophoblasts identified with the FISH LSI probe signals ranged in size, 15-30iim in diameter as measured along the longest axis. Generally, trophoblast diameter as measured along the longest axis can range from 15-49 μm.

FIG. 5b are images from the fluorescence microscope of a) a multinucleated fetal giant cell showing FISH LSI probe signals (multiples of each, more than 2 each) for chromosome 21 and chromosome 13, and b) a multinucleated fetal giant cell showing FISH LSI prob signals (multiples of each) for chromosome 21 and chromosome 18. The multinucleated fetal giant cells identified with multiple FISH LSI probe signals were larger than 50 μm in diameter as measured along the longest axis, and had a roughly elongated shape. Generally, multinucleated fetal giant cell diameter as measured along the longest axis can range from 50 μm-150 μm. Lymphocytes have a diameter in the range of about 5-10 μm.

While the embodiments have been described in connection with the preferred embodiments of the various figures, it is to be understood that other similar embodiments may be used or modifications and additions may be made to the described embodiment for performing the same function without deviating therefrom. Therefore, the disclosed embodiments should not be limited to any single embodiment, but rather should be construed in breadth and scope in accordance with the appended claims.

### Example 6

### Comparative Test on Buffers for Reducing or Preventing Micro-Clots.

Materials

Materials used are listed in Table 16

TABLE 16

| Item | Vendor | Vendor Cat# | Description | Quantity | |
|---|---|---|---|---|---|
| BCT - no | Air-Tite | MV6P | Blood Collection Tube, no | 6 | |
| additive | | | additive | | |
| BioFluidica | BioFluidica | BF020 | Table 11 Formulation | 3 | mL |
| Blood Buffer | | | | | |
| Patent EP | BioFluidica | NA | Table 10 Formulation | 3 | mL |
| 31509181 | | | | | |
| Formulation | | | | | |
| #74 | | | | | |
| BioFluidica | NA | NA | Pre-filled collection tube | 3 | |
| Blood Buffer | | | with BF020 (2021) | | |
| tube | | | | | |
| Healthy donor | NA | NA | Healthy Donor | | |
| whole blood | | | | | |
| CFSE Stained | NA | NA | 100 cells/mL spiked into | | |
| MCF7 Cells | | | same day blood | | |
| EpCAM Ab | Novus Bio | Bulk Order | 1 mg/mL | 3.2 | mL |
| Std EpCAM | BioFluidica | NA | Standard Oligo-EpCAM | 48 | |
| Chips | | | microfluidic chips | | |
| Protein | Thermo Fisher | 89806 | Stabilizes antibodies on | 5.4 | mL |
| Stabilizing | | | the chip | | |
| Solution | | | | | |
| Human Serum | LGC/SeraCare | 1850-0026 | Protein used in stabilizing | 0.6 | mL |
| Albumin | | | cocktail & M. Buffer | | |
| PBS (1x) | Thermo Fisher | 10010049 | Buffer for pre-wash & | 24 | mL |
| | | | rinsing ports | | |
| M. Buffer | BioFluidica | 3000201 | Proprietary Formulation | 2.4 | mL |
| Wash/Elution | BioFluidica | 3000202 | Buffer used for washing | 17.6 | mL |
| Buffer | | | chips & eluting released | | |
| | | | cells | | |
| 2 mL, 96 Deep-well Plate | PlateOne | 1896-2110 | 2 mL 96-Deep well plate | 6 | |
| USER Enzyme | NEB | M5505-Bulk | Enzyme used to release | 4 U/10 | mL |
| | | | captured cells | | |
| Shandon EZ, | Fisher | A78710021 | Double Funnel | 24 | |
| Double Funnel | Scientific | | Cytofunnel | | |
| Fisherbrand | Fisher | 22-037-242 | Double-Ring Microscope | 24 | |
| Double-Ringed | Scientific | | Slide | | |
| Microscope | | | | | |
| Slide | | | | | |
| 7.5% BSA | Sigma-Aldrich | A8412- | Bovine Serum Albumin, | 2.4 | mL |
| | | 100ML | 7.5% in DPBS | | |
| Vectashield, | Vector | H-1500 | VECTASHIELD(R) | 2 | mL |
| Hardset | Laboratories | | HardSet(TM) Antifade | | |
| Mounting | | | Mounting Medium with | | |
| Media | | | DAPI | | |

Each microfluidic chip comprises i) an inlet port, ii) an outlet port, and iii) multiple parallel sinusoidal microchannels in fluid communication with the inlet port and outlet port; wherein said sinusoidal microchannels comprise binding moieties. The binding moieties selectively bind to EpCAM and are immobilized by linkers.

The healthy donor blood is a whole blood sample.

Equipment

Equipment and supplies used are listed in Table 17 and described herein.

TABLE 17

| Description | Model # |
|---|---|
| Hamilton Robot | STARlet |
| Keyence Microscope | BZ-X700 |
| Cytospin | Thermo Scientific Cytospin 4 Cytocentrifuge |
| LiquidScan ™ Module | LS cat#1000202 |

A liquid handling device was used to pump and control fluid through each microfluidic chip. Any suitable liquid handing system to control fluid through the microfluidic chip may be used; such as a liquid handling system comprising i) a pair of automated pipettes corresponding to each microfluidic chip, comprising a) a first automated pipette comprising a first pump, and a first pipette tip containing a sample (e.g. blood sample) and coupled to the inlet port, and; b) a second automated pipette comprising a second pump, and a second pipette tip coupled to the outlet port simultaneous with the first pipette tip coupled to the inlet port; and ii) a controller comprising a non-transitory computer readable medium in communication with the first pump and the second pump, and programmed to command the first pump of the first automated pipette and the second pump of the second automated pipette to control flow of the sample through said one or more microfluidic chips. In an embodiment, the liquid handling system hydrodynamically processes the sample through said one or more microfluidic chips; and isolates at least one target analyte, wherein the at least one target analyte contacts at least one of said binding moieties.

Methods

The comparative test measures the impact of blood additives (BF020 v. Patent EP 315091B1 Formulation #74) on a) capture of target cells in a microfluidic device, b) total cells released (background), and c) microdot formation over time (images).

1. EpCAM microfluidic chips were prepared as follows. 48 Standard oligo-EpCAM microfluidic chips (BioFluidica) were prepared on Jun. 17, 2022. Microfluidic chips with sinusoidal microchannels were surfaced according to a 3-step process: a. Deposition of an enzymatic linker on the plastic surface of the microfluidic device, b. TCEP, a reducing agent to make a sulfide group available to selectively attach the capture antibody, and c. EpCAM (1 mg/mL) derivatized with Sulfo-SMCC, is loaded into the microfluidic chip. The solution is flowed 3 times through the chip to ensure uniform coverage of the antibody on the surface.

2. The EpCAM microfluidic chips were stored with standard Protein stabilizer+10% HSA.

3. Blood was collected in the following tubes in Table 18.

TABLE 18

| | # of | |
|---|---|---|
| Buffer Additive | Tubes | Additives |
| Patent EP 315091B1 | 6 | 6 mL tubes, with 0.6 mL of Formulation |
| Buffer | | #74 Buffer |
| BioFluidica Blood | 3 | 10 mL tube, with 1 mL BF020 (DOM: |
| Buffer | | September 2021) |

The Patent EP 315091B1 buffer composition is based on data in Patent EP 315091 31, formulated as listed in Table 19, consistent with Formulation #74.

TABLE 19

| Component | Stock in BCT (10x) | Working Concentration | |
|---|---|---|---|
| Sucralose | 500 mM | 50 | mM |
| Ala-Gln | 2M | 200 | mM |
| EDTA | 44 mM | 4.4 | mM |
| MES, pH 6.9 | 500 mM | 50 | mM |

The BF020 BioFluidica blood buffer composition was formulated as listed in Table 20.

TABLE 20

| | |
|---|---|
| Stock in | Working |
| BCT (10x) | Concentration |

| Suraclose | 200 | mM | 20 | mM |
|---|---|---|---|---|
| Ficoll PM 70 | 20% | w/v | 2% | |
| Ala-Gln dipeptide | 200 | mM | 20 | mM |
| Human serum albumin (HSA) | 2% | w/v | 0.2% | |
| Adenine | 5.0 | mM | 0.5 | mM |
| EDTA | 44 | mM | 4.4 | mM |
| Bax Channel Inhibitor | 0.10 | mM | 0.01 | mM |

| | | |
|---|---|---|
| Phospho-Buffered-Saline, | 1X | 0.1 X |
| pH 7.2 | | |

In both Tables 19 and 20, the working concentration is the concentration in the blood sample. The Patent EP 315091B1 Formulation #74 buffer does not include Ficoll PM 70, human serum albumin, adenine, and a bax channel inhibitor. See comparison of compositions in Table 21.

TABLE 21

| | BioFluidica | | Patent EP 315091B1 | |
|---|---|---|---|---|
| Component | Blood Buffer | | Formulation #74 Buffer* | |
| Ficoll PM 70 | 2% | w/v | — | |
| Human serum albumin (HSA) | 7.3 | mM | — | |
| Adenine | 5.0 | mM | — | |
| Bax Channel Inhibitor | 0.10 | mM | — | |
| Sucralose | 20 | mM | 50 | mM |
| Ala-Gln | 20 | mM | 20 | mM |
| EDTA | 44 | mM | 4.4 | mM |

| | | |
|---|---|---|
| Buffer System | PBS, pH 7.4 | MES, pH 6.9 |

Blood was collected under the following collection conditions: a. blood is collected over 3 days (June 27, June 28, Jun29), time/date of collection and additive is recorded on each tube; b. collection, blood tubes are stored at room temperature (27° C.) & on a Fisher Scientific constant motion rocker (speed =20) until the time of the experiment; tubes are opened just prior to adding MCF7 cells & then placed on the deck at the time of blood load.

4. 100 CFSE MCF7 cells were added to each mL of blood on the day of the test.

5. Fill 16 tubes (1 tube per chip; 16 chips total) with 1.2 mL of blood each.

6. Process workflow (1×2×5×), regular blood load (8 tubes), custom port rinses including mid BL port rinse:
a. PBS Pre-wash—500 μL, 20 μL/sec;
b. M. Buffer wash—150 μL, 8 μL/sec;
c. 1× Blood load (Forward 500 μL, 13 μL/min);
d. Custom Mid BL PortRinse (removes excess blood from the ports);
e. 1× Blood load (Forward 500 μL, 13 μL/min);
f. Custom Port Rinse Post BL2 using PBS (not HBSS);
g. 2× Wash Buffer—500 μL, 2 μL/sec;
h. Custom Port Rinse Post W1 using PBS;
i. 2× Wash Buffer—500 μL, 2μL/sec;
j. Custom Port Rinse Post W2 using PBS;
k. 2× Wash Buffer—500 μL, 2 μL/sec;
l. Custom Port Rinse Post W3 using PBS;
m. Pause for imaging chips — measure capture of CFSE cells (i. During imaging, heat the heating deck to 37° C.; ii. Image chips using FITC filter for cell capture; iii. Image chips also using Bright Field to identify possible rnicroclots/blockages in channels.);
n. Load USER—40 μL, 1 μL/sec (Total volume of USER made for 16 chips=800 μL; 400 μL/8 chips with dead volume) (one 0.5 mL tube per robot) (i. DI Water=400 μL; ii. CutSmart Buffer=80 μL; iii. USER enzyme=320 μL; iv. After preparing the USER in a 1.5 mL tube, vortex the tube 3× for 3 seconds and then spin down for 10 seconds with microfuge; v. only place chips back on the deck when USER is ready to be loaded to avoid drying the chips out on the heating deck; vi. Rehydrate ports with a drop of PBS before loading)
o. Incubate at 37° C. for 30 min.
p. Elute with Elution Buffer—300 μL, 5μL/sec
q. Finished: Image chips—measure how many cells were released (Image chips using FITC filter (cells remaining); Image chips using Bright Field (blocked channels/clots remaining))
r. Eluates are put onto Cytospin slides for target & total cell evaluation. (i. double funnel cytospin holders are cleaned using compressed air cans & a double ring slide is placed inside & secured; ii. Slides are placed in the cytospin (careful to make sure they are balanced); iii. 100 μL of 7.5% BSA is added to each funnel & then spun at 740 rpm for 1 min to wet the filter paper; iv. 300 μL of each eluate are placed in the corresponding funnel & samples are deposited at 740 rpm for 4 min; v. slides are dried & then covered with Hardset Mounting media with DAPI; vi. after drying for a minimum of 30 min the slides are imaged using the Keyence microscope in both green (CFSE˜MCF7) & DAPI (all cells); vii. Images are stitched & counted using the Keyence analysis software.

Table 22 reflects the process workflow described above. (Robot Run Conditions (1×2×5×)

TABLE 22

| | | | | Blood | | Wash | | | |
|---|---|---|---|---|---|---|---|---|---|
| | | | | load | Wash | Buffer | Wash | | |
| | PBS | M. | | reverse | Buffer** | ** wash | Buffer** | USER | -Elution |
| | Wash | Buffer | Blood* | ** | wash 1 | 2 | wash 3 | Elution | wash |
| Aspirate | 5000 | 1500 | 5000 | 5000 | 5000* | 5000* | 5000* | 400 | 3000 |
| Volume | | | | | | | | | |
| (0.1 L) | | | | | | | | | |
| Aspirate | 200 | 80 | 10 | 10 | 20 | 20 | 20 | 10 | 50 |
| Speed (0.1 | | | | | | | | | |
| μL/sec) | | | | | | | | | |
| Dispense | 5000 | 1500 | 5000 | 5000 | 5000* | 5000* | 5000* | 400 | 3000 |
| Vol (0.1 μL) | | | | | | | | | |
| Dispense | 200 | 80 | 10 | 10 | 20 | 20 | 20 | 10 | 50 |
| Speed (0.1 | | | | | | | | | |
| μL/sec) | | | | | | | | | |
| Aspirate | 2025 | 2017 | 2025 | 2025 | 2025 | 2025 | 2025 | 2017 | 2025 |
| Height (0.1 | | | | | | | | | |
| mm) | | | | | | | | | |
| Dispense | 2025 | 2017 | 2025 | 2025 | 2025 | 2025 | 2025 | 2017 | 2025 |
| Height (0.1 | | | | | | | | | |
| mm) | | | | | | | | | |
| Blowout | 0 | 0 | 0 | 0 | 0 | 0 | 0 | 0 | 0 |
| Volume | | | | | | | | | |
| (0.1 mL) | | | | | | | | | |
| Settling | 0 | 0 | 0 | 0 | 0 | 0 | 0 | 0 | 0 |
| Time after | | | | | | | | | |
| Aspirating | | | | | | | | | |
| (0.1 s) | | | | | | | | | |
| Settling | 0 | 0 | 0 | 0 | 0 | 0 | 0 | 0 | 0 |
| Time after | | | | | | | | | |
| Dispensing | | | | | | | | | |
| (0.1 s) | | | | | | | | | |
| Global | 45 | 75 | 45 | 45 | 10 | 10 | 10 | 1800 | 120 |
| Settling | | | | | | | | | |
| Time (0.1 | | | | | | | | | |
| s) | | | | | | | | | |
| Mix | 0 | 0 | 0 | 0 | 0 | 0 | 0 | 0 | 0 |
| Volume | | | | | | | | | |
| (mL) | | | | | | | | | |
| Mix Cycles | 0 | 0 | 0 | 0 | 0 | 0 | 0 | 0 | 0 |
| Pulse | 10 | 10 | 150 | 150 | 15 | 15 | 15 | 1 | 1 |
| Number | | | | | | | | | |
| Pulse | 0.1 | 0.1 | 1.2 | 1.2 | 0.1 | 0.1 | 0.1 | 0 | 0 |
| Pause (sec) | | | | | | | | | |
| Ramp | 1 | 1 | 1 | 1 | 1 | 1 | 1 | 1 | 1 |
| Pulse | | | | | | | | | |
| Count | | | | | | | | | |
| Flow Cycles | 1 | 1 | 1 | 1 | 1 | 1 | 1 | 1 | 1 |
| Direction | Back- | Forward | Forward | Back- | Forward | Forward | Forward | Forward | Forward |
| of Flow | wards | | | wards | | | | | |
| Over | 0 | 0 | 0 | 0 | 0 | 0 | 0 | 0 | 0 |
| Aspirate | | | | | | | | | |
| Dual | 0 | 0 | 0 | 0 | 0 | 0 | 0 | 0 | 0 |
| Collection | | | | | | | | | |
| Source | 1 | 9 | 121 | 121 | 97 | 97 | 97 | 152 | 41 |
| Tube | | | | | | | | | |
| Position | | | | | | | | | |
| Destination | 209 | 209 | 209 | 209 | 209 | 209 | 209 | 233 | 233 |
| Tube | | | | | | | | | |
| Position | | | | | | | | | |
| Pause | no | no | yes | yes | yes | yes | yes | no | no |
| BackFlow | 100 | 100 | 100 | 100 | 0 | 0 | 0 | 0 | 0 |
| Pre (0.1 | | | | | | | | | |
| mL) | | | | | | | | | |
| BackFlow | 450 | 500 | 500 | 500 | 20 | 20 | 20 | 20 | 0 |
| Post (0.1 | | | | | | | | | |
| mL) | | | | | | | | | |
| Tip Type | 1000 | 300 | 1000 | 1000 | 300 | 300 | 300 | 300 | 1000 |

Results:

Results for 2 chips per condition: 1. Capture of target cells was impacted by the presence of microclots. After 2 days of storage the capture for the Patent EP 315091B 1 Formulation #74 buffer was 50% of the BioFluidica blood buffer capture. 2. The presence of microclots leads to an increase of nonspecific cells being trapped in the channels. And background cells that are released from the microfluidic chip—resulting in 3.5× higher background for the Patent EP 315091B1 Formulation #74 buffer even after 1.5 hrs of storage. See Table 23.

TABLE 23

| | % Capture | Background |
|---|---|---|
| Buffer Additives | (Day 2) | (Day 2) |
| Biofluidica Blood Buffer | 100 | 197 |
| Patent EP 315091B1 Buffer* | 55 | 606 |

3. Examining the brightfield images (shown in next slides), the BioFluidica buffer does not show any microclots in the microfluidic chip even after 48 hrs of storage. However, the Patent EP 315091B1 Formulation #74 buffer has ˜50% blockage on the top manifold & ˜75-80% blockage on the bottom manifold due to microclots covering the channels.

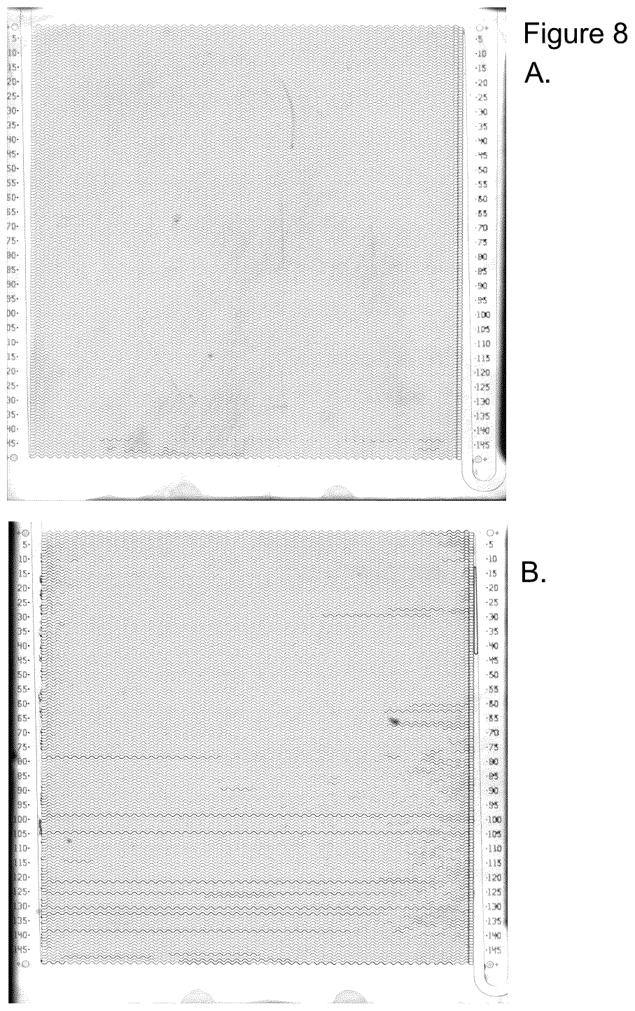

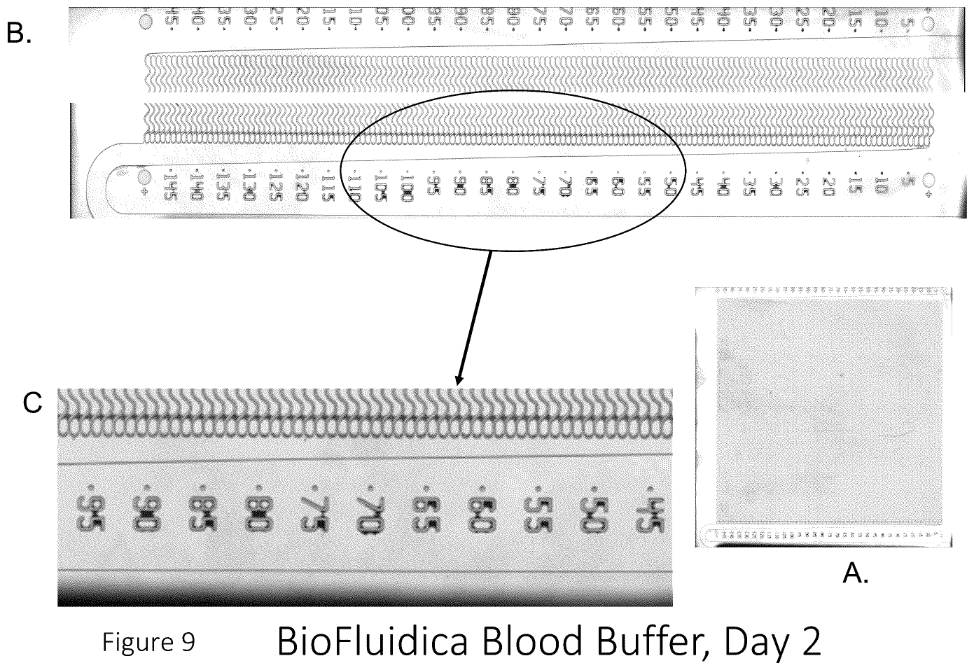

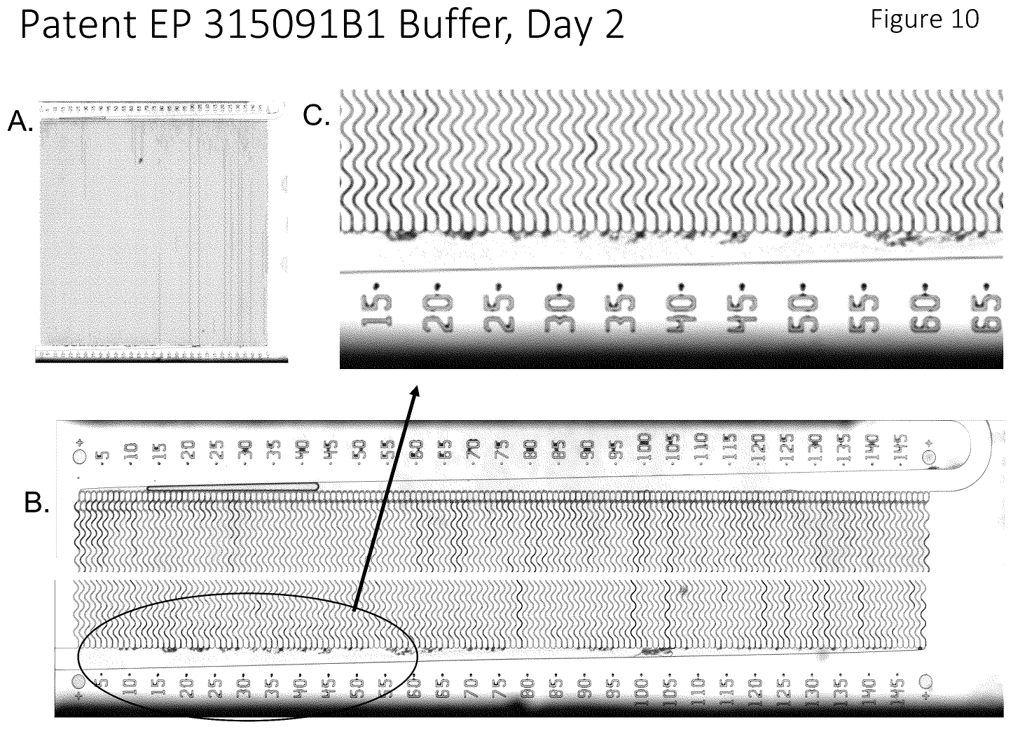

See FIGS. 8, 9, 10. FIG. 8 shows full chip images of a chip that utilized the BioFluidica blood buffer (A) and a chip that utilized the Patent EP 315091131 Formulation #74 buffer (B). FIG. 9 shows a full chip image of a chip that utilized the BioFluidica blood buffer (A), an enlarged image of the inlet channel and entry of the sinusoidal microchannels (B, top) and the outlet channel and exit of the sinusoidal microchannels (B, bottom), and a further enlarged image of the inlet channel and entry of the sinusoidal microchannels (C). As shown in the images, the microfluidic chip is free of microclots and clogged microchannels. FIG. 10 shows a full chip image of a chip that utilized the Patent EP 315091B 1 Formulation #74 blood buffer (A), an enlarged image of the inlet channel and entry of the sinusoidal microchannels (B, top) and the outlet channel and exit of the sinusoidal microchannels (B, bottom), and a further enlarged image of the inlet channel and entry of the sinusoidal microchannels (C). As shown in the images, the microfluidic chip has microclots at the entry of the sinusoidal microchannels and clogged microchannels (shown as dark sinusoidal microchannels).

### REFERENCES

Janina Walknowska, Felix A. Conte, M. G. Practical and theoretical implications of fetal-maternal lymphocyte transfer. Lancet (1969). doi:10.1016/S0140-6736(69)91642-0

Chen, F. et al. Isolation and whole genome sequencing of fetal cells from maternal blood towards the ultimate non-invasive prenatal testing. Prenat. Diagn. (2017). doi:10.1002/pd.5186

Grati, F. R. Chromosomal Mosaicism in Human Feto-Placental Development: Implications for Prenatal Diagnosis. J. Clin. Med. 3, 809-837 (2014).

Hou, S. et al. Imprinted NanoVelcro Microchips for Isolation and Characterization of Circulating Fetal Trophoblasts: Toward Noninvasive Prenatal Diagnostics. ACS Nano (2017). doi:10.102 1/a csnano.7b03073

Vestergaard, E. M. et al. On the road to replacing invasive testing with cell-based NIPT: Five clinical cases with aneuploidies, microduplication, unbalanced structural rearrangement, or mosaicism. Prenat. Diagn. (2017). doi:10.1002/pd.5150

Noninvasive Prenatal Testing for Fetal Aneuploidy: Committee Opinion. The American College of Obstetricians and Gynecologists and the American Congress of Obstetricians and Gynecologists, The Society for Maternal-Fetal Medicine Number 545 (2012).

What is claimed:

1. A method of isolating fetal cells from a sample from a pregnant subject comprising:
(a) providing an isolation system comprising:
one or more microfluidic chips, wherein each microfluidic chip comprises:
i) an inlet port,
ii) an outlet port, and
iii) multiple parallel sinusoidal microchannels in fluid communication with the inlet port and outlet port; wherein said sinusoidal microchannels comprise at least one of: binding moieties that selectively bind to EpCAM, binding moieties that selectively bind to CD105, and/or binding moieties that selectively bind to CD141;
a liquid handling system comprising:
i) a pair of automated pipettes corresponding to each microfluidic chip, comprising:
a. a first automated pipette comprising a first pump, and a first pipette tip containing a sample from a pregnant subject and coupled to the inlet port, wherein the sample is maternal whole blood, has not been processed to remove maternal cells, and comprises at least one fetal cell and;
b. a second automated pipette comprising a second pump, and a second pipette tip coupled to the outlet port; and
ii) a controller comprising a non-transitory computer readable medium in communication with the first pump and the second pump, and programmed to command the first pump of the first automated pipette and the second pump of the second automated pipette to control flow of the sample through said one or more microfluidic chips; and
(b) hydrodynamically processing the sample through said one or more microfluidic chips; and
(c) isolating at least one fetal cell, wherein the at least one fetal cell contacts at least one of said binding moieties, and wherein the method does not comprise removing maternal cells prior to isolation of the at least one fetal cell.

2. The method of claim 1, wherein the isolation system comprises two or more microfluidic chips, wherein each first pipette tip of each first automated pipette of each pair of automate pipettes corresponding to each chip comprises the same sample, wherein hydrodynamically processing the sample through said two or more microfluidic chips comprises flowing a portion of the same sample through each microfluidic chip, and the method comprises isolating an average of 0.5 fetal cells per milliliter of sample.

3. The method of claim 1, wherein the method comprises recovering at least 1 fetal cell in the presence of at least 500 maternal cells, at least 750 maternal cells, or at least 1000 maternal cells.

4. The method of claim 1, wherein the isolation system comprises eight microfluidic chips and eight pairs of automated pipettes with each respective first pipette tip containing at least 1 milliliter of the same sample, and wherein hydrodynamically processing the sample through said eight microfluidic chips comprises flowing the at least 1 ml of sample in each first pipette tip through the corresponding microfluidic chip.

5. The method of claim 1, wherein the binding moieties are antibodies, aptamers, affimers, or haptens immobilized to the interior surface of the sinusoidal microchannels.

6. The method of claim 1, wherein said microchannels comprise a synergistic combination of binding moieties that selectively bind to EpCAM and binding moieties that selectively bind to CD141.

7. The method of claim 6, wherein the method comprises isolating at least 3 fetal cells per milliliter of sample.

8. The method of claim 6, wherein the isolation system comprises two or more microfluidic chips, wherein each first pipette tip of each first automated pipette of each pair of automate pipettes corresponding to each chip comprises the same sample, wherein hydrodynamically processing the sample through said two or more microfluidic chips comprises flowing a portion of the same sample through each microfluidic chip, and the method comprises isolating at least an average of 3 fetal cells per milliliter of sample.

9. The method of claim 1, wherein said microchannels comprise a synergistic combination of binding moieties that selectively bind to EpCAM and binding moieties that selectively bind to CD105.

10. The method of claim 9, wherein the method comprises isolating at least 2 fetal cells per milliliter of sample.

11. The method of claim 9, wherein the isolation system comprises two or more microfluidic chips, wherein each first pipette tip of each first automated pipette of each pair of automate pipettes corresponding to each chip comprises the same sample, wherein hydrodynamically processing the sample through said two or more microfluidic chips comprises flowing a portion of the same sample through each microfluidic chip, and the method comprises isolating at least an average of 2 fetal cells per milliliter of sample.

12. The method of claim 1, wherein the fetal cells are fetal trophoblastic cells.

13. The method of claim 1, wherein the fetal cells are extravillous trophoblast cells.

14. The method of claim 1, wherein the sample is taken during gestation week 8, 9, 10, 11, 12, 13, 14, 15, 16, 17, or 18.

15. The method of claim 1, wherein the microchannels have a microchannel width in the range of about 15 μm to about 45 μm and a microchannel height in the range of about 100 μm to about 160 m.

16. The method of claim 1, wherein the sample is maternal whole blood admixed with a whole blood buffer composition comprising a ficollpolysaccharide and a halogenated sucrose derivative.

17. The method of claim 1, wherein the sample is maternal whole blood admixed with a dual whole blood and fixative buffer composition comprising
a ficollpolysaccharide,
a halogenated sucrose derivative,
an anticoagulant,
at least one of glyoxal or paraformaldehyde or formaldehyde or a formal acetic acid or a formal saline or a phosphate formalin or a formalin calcium, and
a pH buffer.

18. The method of claim 1, wherein hydrodynamically processing the sample through said one or more microfluidic chips comprises applying a pulsative flow.

19. The method of claim 18, wherein the overall flow rate is in the range of 5 μl to 30 μl per minute, 10 μl to 25 μl per minute, 10 μl to 30 μl per minute, 5 μl to 15 μl per minute, 5 μl to 10 μl per minute, or 10 μl to 15 μl per minute.

20. A method of isolating multinucleated fetal giant cells from a sample from a pregnant subject comprising:
(a) providing an isolation system comprising:
one or more microfluidic chips, wherein each microfluidic chip comprises:
i) inlet port,
ii) an outlet port, and
iii) multiple parallel sinusoidal microchannels in fluid communication with the inlet port and outlet port; wherein said sinusoidal microchannels have a channel width in the range of about 15 μm to about 45 μm and a channel height in the range of about 100 μm to about 150 μm, and wherein said sinusoidal microchannels comprise binding moieties that selectively bind to a surface marker of fetal cells;
a liquid handling system comprising:
i) a pair of automated pipettes corresponding to each microfluidic chip, comprising:
a. a first automated pipette comprising a first pump, and a first pipette tip containing a sample from a pregnant subject and coupled to the inlet port, wherein the sample from a pregnant subject is maternal whole blood and comprises at least one multinucleated fetal giant cell;
b. a second automated pipette comprising a second pump, and a second pipette tip coupled to the outlet port; and
ii) a controller comprising a non-transitory computer readable medium in communication with the first pump and the second pump, and programmed to command the first pump of the first automated pipette and the second pump of the second automated pipette to control flow of the sample through said one or more microfluidic chips; and
(b) hydrodynamically processing the sample through said one or more microfluidic chips; and
(a) isolating at least one multinucleated fetal giant cell, wherein the at least one multinucleated fetal giant cell contacts at least one of said binding moieties and wherein the at least one multinucleated fetal giant cell is 50 to 150 μm in diameter as measured along the longest axis.

21. The method of claim 20, wherein said sinusoidal microchannels have a channel width in the range of about 20 μm to about 25 μm and a channel height in the range of about 140 μm to about 150 μm.

22. The method of claim 20, wherein the sample has not been processed to removed maternal cells, and the method does not comprise removing maternal cells prior to isolation of the at least one fetal cell.

23. The method of claim 20, wherein hydrodynamically processing the sample through said one or more microfluidic chips comprises applying a pulsative flow.

24. The method of claim 23, wherein the overall flow rate is in the range of 5 μl to 30 μl per minute, 10 μl to 25 μl per minute, 10 μl to 30 μl per minute, 5 μl to 15 μl per minute, 5 μl to 10 μl per minute, or 10 μl to 15 μl per minute.

25. The method of claim 23, wherein applying a pulsative flow comprises 300 pulses per milliliter of maternal sample and a repetitive flow pattern comprising an increase in flow rate to 1 μl/sec for 100 milliseconds and a pause for 100 milliseconds.

26. The method of claim 23, wherein applying a pulsative flow comprises 100 to 400 pulses per milliliter of maternal sample and a repetitive flow pattern comprising an increase in flow rate to a flow rate in a range of 1-5 μl/sec for 100-200 milliseconds and a pause for a period of time in a range from 100 milliseconds to 1 second.

27. The method of claim 20, wherein the binding moieties are anti-EpCAM antibodies.

28. The method of claim 20, wherein the method further comprises identifying the at least one multinucleated fetal giant cell by staining with anti-EpCAM antibodies, or by staining the nuclei or DNA with a Hoechst stain.

29. The method of claim 20, wherein the method further comprises identifying the at least one multinucleated fetal giant cell by FISH probe signals. The method of claim 20, wherein the sample is maternal whole blood admixed with a whole blood buffer composition comprising a ficollpolysaccharide and a halogenated sucrose derivative.

31. The method of claim 20, wherein the sample is maternal whole blood admixed with a dual whole blood and fixative buffer composition comprising
a ficollpolysaccharide,
a halogenated sucrose derivative,
an anticoagulant,
at least one of glyoxal or paraformaldehyde or formaldehyde or a formal acetic acid or a formal saline or a phosphate formalin or a formalin calcium, and a pH buffer.

32. The method of claim 1, wherein the binding moieties are immobilized by linkers.

33. The method of claim 1, wherein the method further comprises degrading the linkers and recovering the fetal cells in an eluate.

34. The method of claim 33, wherein the method further comprises lysing the recovered fetal cells in the eluate to obtain fetal nucleic acids. The method of claim 1, wherein the method further comprises lysing isolated cells and recovering fetal nucleic acids.

36. The method of claim 1, wherein the method further comprises
recovering isolated fetal cells or fetal DNA in an eluate;
providing one or more microfluidic chips for detection or analysis of fetal cells or fetal DNA, wherein each microfluidic chip comprises an inlet port, an outlet port, and microchannels in fluid communication with the inlet port and outlet port;
providing a pair of automated pipettes corresponding to each microfluidic chip, comprising a first automated pipette comprising a first pump, and a first pipette tip containing the eluate or a solution comprising the isolated fetal cells or fetal DNA and coupled to the inlet port; a second automated pipette comprising a second pump, and a second pipette tip coupled to the outlet port; and
wherein the controller is further programmed to command the first pump of the first automated pipette and the second pump of the second automated pipette to control flow of the eluate or the solution through the microfluidic chip; and
hydrodynamically processing the eluate or the solution through said one or more microfluidic chips.

37. The method of claim 1, wherein recovery of the fetal cells occurs in less than 2 hours.

38. The method of claim 33, wherein the method further comprises using the fetal cells from the eluate for cell-based analysis.

39. The method of claim 38, wherein the cell-based analysis is ICC, or fluorescent in situ hybridization (FISH).

40. The method of claim 35, wherein the method further comprises using the fetal nucleic acids for nucleic acid based analysis.

41. The method of claim 40, wherein the nucleic acid based analysis is qPCR, NGS, amplification, DNA sequencing, or RNA sequencing, northern blotting, southern blotting, or microarray analysis.

42. The method of claim 33, wherein the method further comprises using the fetal cells from the eluate to detect autosomal and sex chromosome aneuploidies, microdeletions, or duplications.

43. The method of claim 35, wherein the method further comprises using the fetal nucleic acids to detect a single gene disorder.

44. The method of claim 20, wherein the binding moieties are immobilized by linkers. The method of claim 20, wherein the method further comprises degrading the linkers and recovering the fetal cells in an eluate.

46. The method of claim 45, wherein the method further comprises lysing the recovered fetal cells in the eluate to obtain fetal nucleic acids.

47. The method of claim 20, wherein the method further comprises lysing isolated cells and recovering fetal nucleic acids.

48. The method of claim 20, wherein the method further comprises recovering isolated fetal cells or fetal DNA in an eluate;
providing one or more microfluidic chips for detection or analysis of fetal cells or fetal DNA, wherein each microfluidic chip comprises an inlet port, an outlet port, and microchannels in fluid communication with the inlet port and outlet port;
providing a pair of automated pipettes corresponding to each microfluidic chip, comprising a first automated pipette comprising a first pump, and a first pipette tip containing the eluate or a solution comprising the isolated fetal cells or fetal DNA and coupled to the inlet port; a second automated pipette comprising a second pump, and a second pipette tip coupled to the outlet port; and
wherein the controller is further programmed to command the first pump of the first automated pipette and the second pump of the second automated pipette to control flow of the eluate or the solution through the microfluidic chip; and
hydrodynamically processing the eluate or the solution through said one or more microfluidic chips.

49. The method of claim 20, wherein recovery of the fetal cells occurs in less than 2 hours.

50. The method of claim 45, wherein the method further comprises using the fetal cells from the eluate for cell-based analysis.

51. The method of claim 50, wherein the cell-based analysis is ICC, or fluorescent in situ hybridization (FISH).

52. The method of claim 47, wherein the method further comprises using the fetal nucleic acids for nucleic acid based analysis.

53. The method of claim 40, wherein the nucleic acid based analysis is qPCR, NGS, amplification, DNA sequencing, or RNA sequencing, northern blotting, southern blotting, or microarray analysis.

54. The method of claim 45, wherein the method further comprises using the fetal cells from the eluate to detect autosomal and sex chromosome aneuploidies, microdeletions, or duplications.

55. The method of claim 47, wherein the method further comprises using the fetal nucleic acids to detect a single gene disorder.

56. A whole blood buffer composition comprising a ficoll polysaccharide and a halogenated sucrose derivative.

57. The composition of claim 56, wherein the composition further comprises at least one of a human serum albumin or a bovine serum albumin.

58. The composition of claim 56, wherein the composition further comprises a cell apoptosis inhibitor.

59. An article of manufacture comprising the composition of claim 56.

60. The article of manufacture of claim 59, wherein the article of manufacture is a blood collection device.

61. The article of manufacture of claim 59, wherein the article of manufacture is a blood collection tube.

62. A method of stabilizing analytes in a whole blood sample at ambient temperatures comprising admixing a whole blood sample ex vivo with a composition of claim 56.

63. A method of inhibiting microclotting of a whole blood sample in a microfluidic device comprising admixing a whole blood sample ex vivo with a composition of claim 56 prior to introduction of the whole blood sample into a microfluidic device comprising microchannels, and hydrodynamically processing the admixed whole blood sample through the microfluidic chip, wherein microclotting in the microfluidic device is inhibited.

64. The method of claim 63, wherein the microchannels have a channel width or channel height in the range of about 10 to about 200 μm.

65. The method of claim 64, wherein the microfluidic chip comprises:
iv) an inlet port,
v) an outlet port, and
vi) microchannels in fluid communication with the inlet port and outlet port.

66. The method of claim 65, further comprising:
(d) providing a liquid handling system for hydrodynamically processing the admixed whole blood sample through the microfluidic chip, wherein the liquid handling system comprises:
ii) a pair of automated pipettes comprising:
a. a first automated pipette comprising a first pump, and a first pipette tip containing the admixed whole blood sample, wherein the first pipette tip is coupled to the inlet port, and;
b. a second automated pipette comprising a second pump, and a second pipette tip coupled to the outlet port simultaneously with the first pipette tip coupled to the inlet port; and
iii) a controller comprising a non-transitory computer readable medium in communication with the first pump and the second pump, and programmed to command the first pump of the first automated pipette and the second pump of the second automated pipette to control flow of the admixed whole blood sample through the microfluidic chip.

67. The method of claim 64, further comprising hydrodynamically processing the admixed whole blood sample through the microfluidic chip with a pump.

68. The method of claim 67, wherein the pump is a syringe pump.
